(19) United States
(12) Patent Application Publication
(10) Pub. No.: US 2026/0174788 A1
(43) Pub. Date: Jun. 25, 2026

(54) GEMCITABINE FOR USE IN METHODS OF TREATING HIGH-RISK NON-MUSCLE INVASIVE BLADDER CANCER UNRESPONSIVE TO BACILLUS CALMETTE-GUÉRIN THERAPY

(71) Applicant: TARIS Biomedical LLC, Lexington, MA (US)

(72) Inventors: Christopher J. CUTIE, Lexington, MA (US); Kiran PATEL, Chesterbrook, PA (US); Hussein SWEITI, Royersford, PA (US)

(21) Appl. No.: 19/355,699

(22) Filed: Oct. 10, 2025

Related U.S. Application Data
(63) Continuation of application No. PCT/US2024/023758, filed on Apr. 9, 2024.
(60) Provisional application No. 63/495,282, filed on Apr. 10, 2023.
(60) Provisional application No. 63/499,048, filed on Apr. 28, 2023.
(60) Provisional application No. 63/583,747, filed on Sep. 19, 2023.
(60) Provisional application No. 63/588,925, filed on Oct. 9, 2023.
(60) Provisional application No. 63/618,107, filed on Jan. 5, 2024.
(60) Provisional application No. 63/623,018, filed on Jan. 19, 2024.
(60) Provisional application No. 63/554,080, filed on Feb. 15, 2024.

(51) Int. Cl. A61K 31/7068; A61P 35/00

(52) U.S. Cl. CPC A61K 31/7068; A61P 35/00

(57) ABSTRACT

The present invention provides methods for treatment of high-risk non-muscle invasive bladder cancer that is unresponsive to BCG therapy by locally administering gemcitabine to the bladder. Also provided herein are kits and articles of manufacture for treating high-risk non-muscle invasive bladder cancer that is unresponsive to BCG.

### CROSS-REFERENCE TO RELATED APPLICATIONS

This application is a continuation of International Application No. PCT/US2024/023758, filed Apr. 9, 2024, which claims priority from U.S. provisional application 63/554,080, filed Feb. 15, 2024, U.S. provisional application 63/623,018, filed Jan. 19, 2024, U.S. provisional application 63/618,107, filed Jan. 5, 2024, U.S. provisional application 63/588,925, filed Oct. 9, 2023, U.S. provisional application 63/583,747, filed Sep. 19, 2023, U.S. provisional application 63/499,048, filed Apr. 28, 2023, and U.S. provisional application 63/495,282, filed Apr. 10, 2023, the contents of each of which are incorporated by reference in their entirety.

### FIELD

The present disclosure relates to methods of treating high-risk non-muscle invasive bladder cancer that is unresponsive to Bacillus Calmette-Guérin (BCG) therapy comprising continuous delivery of gemcitabine to the bladder. The present disclosure also relates to gemcitabine for use in methods of treating high-risk non-muscle invasive bladder cancer that is unresponsive to Bacillus Calmette-Guérin (BCG) therapy wherein the method comprises continuous delivery of gemcitabine to the bladder. The present disclosure also relates to minitablets comprising gemcitabine for use in methods of treating high-risk non-muscle invasive bladder cancer that is unresponsive to Bacillus Calmette-Guérin (BCG) therapy wherein the method comprises continuous delivery of gemcitabine to the bladder. The present disclosure further relates to gemcitabine for use with an intravesical drug delivery system, in methods of treating high-risk non-muscle invasive bladder cancer that is unresponsive to Bacillus Calmette-Guérin (BCG) therapy wherein the method comprises continuous delivery of gemcitabine to the bladder. The present disclosure further relates to gemcitabine for use in methods of treating high-risk non-muscle invasive bladder cancer that is unresponsive to Bacillus Calmette-Guérin (BCG) therapy, in combination with an intravesical device, wherein the method comprises continuous delivery of gemcitabine to the bladder by an intravesical drug delivery system comprising the intravesical device.

### REFERENCE TO AN ELECTRONIC SEQUENCE LISTING

The contents of the electronic sequence listing (761662002401seqlist.xml; Size: 11,760 bytes; and Date of Creation: Oct. 6, 2025) is herein incorporated by reference in its entirety.

### BACKGROUND OF THE INVENTION

Bladder cancer is a significant medical problem, and currently available treatment options are unsatisfactory for a number of reasons. According to the Pharma Intelligence database (Citeline), as of the filing date of this application, there had been more than 1300 clinical trials looking to identify safe and effective treatments for bladder cancer. Of those trials, more than 1000 either failed to meet their outcome, were terminated, or were otherwise discontinued. There remains a significant unmet medical need for patients with bladder cancer and the subject matter described and claimed herein is a significant step to helping meet that need.

Systemic neoadjuvant chemotherapy, transurethral resection of bladder tumor (TURBT) followed by intravesical treatment with Bacillus Calmette-Guérin (BCG) therapy, and radical cystectomy are among the currently available treatment options for bladder cancer patients, depending on the stage of bladder cancer.

Systemic chemotherapy is associated with significant toxicity, risks overtreatment in a significant proportion of patients, and up to 80% of patients may refuse or be ineligible for neoadjuvant and/or adjuvant regimens (Haseebuddin et al. J Urol. 2015; 139 (4S): e852-e853). Furthermore, while BCG therapy can be successful at preventing early tumor recurrences in patients with high-risk non-muscle invasive bladder cancer (NMIBC), most patients do not maintain sustained remissions (Shelley et al. Cochrane Database Syst Rev. 2000; (4): CD001986). BCG treatment eventually fails in up to 50% of patients; almost 50% of these failures occur within the first 6 months (Lightfoot et al. Scientific World J. 2011 Mar. 7; 11:602-613). Thus, many patients will ultimately develop BCG-unresponsive disease.

Radical cystectomy is associated with high morbidity (up to 60%) and negatively affects health related quality of life. These complications occur even in high-volume centers of excellence and regardless of surgical technique or approach (Shabsigh et al. Eur Urol. 2009; 55:164-176). The procedure itself has a mortality rate of 3% (Stein et al. J Clin Oncol. 2001; 19:666-67). Hence, some patients refuse surgery. Additionally, some patients are medically unfit for surgery due to such factors as age, functional status, American Society of Anesthesiologists Class Score, co-morbidities, and body mass index.

Thus, there exists a significant clinical need for alternative treatment options such as those that allow individuals to retain their bladders. The present application addresses these and other needs.

The disclosures of all publications, patents, patent applications and published patent applications referred to herein are hereby incorporated herein by reference in their entirety.

### BRIEF DESCRIPTION OF THE INVENTION

The present invention in various aspects provides methods for treatment of high-risk non-muscle invasive bladder cancer that is unresponsive to BCG therapy by locally administering gemcitabine to the bladder. In some aspects, provided herein are kits and articles of manufacture for treating high-risk non-muscle invasive bladder cancer that is unresponsive to BCG.

In some embodiments, provided herein is a method of treating high-risk non-muscle invasive bladder cancer (HR-NMIBC) that is unresponsive to intravesical Bacillus Calmette-Guerin (BCG) therapy in an individual comprising administering to the individual about 225 mg of gemcitabine about every three weeks (Q3W) for at least about 24 weeks, wherein such treatment results in a complete response rate in a population of patients who have received such treatment of at least 50%.

In some embodiments, provided herein is a method of bladder sparing in an individual having high-risk non-muscle invasive bladder cancer (HR-NMIBC) that is unresponsive to intravesical Bacillus Calmette-Guerin (BCG) therapy comprising administering to the individual about 225 mg of gemcitabine about every three weeks (Q3W) for at least about 24 weeks, wherein the individual does not receive a radical cystectomy.

In some embodiments, provided herein is a method of treating high-risk non-muscle invasive bladder cancer (HR-NMIBC) that is unresponsive to intravesical Bacillus Calmette-Guerin (BCG) therapy in an individual who is eligible for radical cystectomy, the method comprising administering to the individual about 225 mg of gemcitabine about every three weeks (Q3W) for at least about 24 weeks.

In some embodiments, provided herein is a method of treating high-risk non-muscle invasive bladder cancer (HR-NMIBC) that is unresponsive to intravesical Bacillus Calmette-Guerin (BCG) therapy in an individual comprising inserting an intravesical drug delivery system comprising about 225 mg gemcitabine into the bladder of the individual and removing the intravesical drug delivery system about three weeks later for at least about 24 weeks, wherein such treatment results in a complete response rate in a population of patients who have received such treatment of at least 50%.

In some embodiments, provided herein is a method of increasing the complete response rate for treating high-risk non-muscle invasive bladder cancer (HR-NMIBC) that is unresponsive to intravesical Bacillus Calmette-Guerin (BCG) therapy in an individual comprising administering to the individual about 225 mg of gemcitabine about every three weeks (Q3W) for at least about 24 weeks, wherein such treatment results in a complete response rate that is at least 1.5 fold the complete response rate achieved by valrubicin in a population of patients who have received such treatment.

In some embodiments, provided herein is a method of treating Bacillus Calmette-Guerin (BCG) unresponsive high-risk non-muscle invasive bladder cancer (HR-NMIBC) with carcinoma in situ (CIS) or Tis with or without papillary tumors in an individual comprising administering to the bladder of the individual about 225 mg of gemcitabine about every three weeks (Q3W) for at least about 24 weeks, wherein such treatment results in a complete response rate in a population of patients who have received such treatment of at least 50%.

In some embodiments, provided herein is a method of treating Bacillus Calmette-Guerin (BCG) unresponsive high-risk non-muscle invasive bladder cancer (HR-NMIBC) with carcinoma in situ (CIS) with or without papillary tumors in an individual comprising administering to the bladder of the individual about 225 mg of gemcitabine about every three weeks (Q3W) for at least about 24 weeks, wherein the individual has a complete response.

In some embodiments, provided herein is a method of inducing a complete response (CR) rate of about 72.7% in a patient population with high-risk non-muscle invasive bladder cancer (HR-NMIBC) that is unresponsive to intravesical Bacillus Calmette-Guerin (BCG) therapy, the method comprising inserting a TAR-200 intravesical drug delivery system comprising about 225 mg gemcitabine into the bladder of an individual with HR-NMIBC, and removing the TAR-200 about three weeks later for at least about 24 weeks.

In some embodiments, provided herein is a method of inducing a complete response (CR) rate of at least 50% in a patient population with high-risk non-muscle invasive bladder cancer (HR-NMIBC) that is unresponsive to intravesical Bacillus Calmette-Guerin (BCG) therapy, the method comprising i) inserting a TAR-200 intravesical drug delivery system comprising about 225 mg gemcitabine into the bladder of an individual with HR-NMIBC, and ii) removing the TAR-200 about three weeks later, wherein i) and ii) are repeated for at least about 24 weeks.

In some embodiments, provided herein is a method of treating high-risk non-muscle invasive bladder cancer (HR-NMIBC) that is unresponsive to intravesical Bacillus Calmette-Guerin (BCG) therapy in an individual comprising administering to the individual about 225 mg of gemcitabine about every three weeks (Q3W) for at least about 24 weeks.

In some embodiments, provided herein is a method of treating high-risk non-muscle invasive bladder cancer (HR-NMIBC) that is unresponsive to intravesical Bacillus Calmette-Guerin (BCG) therapy in an individual comprising administering about 225 mg of gemcitabine to the bladder of the individual every three weeks (Q3W) for at least about 24 weeks during eight administration periods, wherein each administration period comprises a delivery period of at least seven days when gemcitabine is released from an intravesical drug delivery system, wherein the concentration of gemcitabine in the urine of the individual is at least 4 μg/mL during each delivery period.

In some embodiments, provided herein is the method of treating high-risk non-muscle invasive bladder cancer (HR-NMIBC) that is unresponsive to intravesical Bacillus Calmette-Guerin (BCG) therapy in an individual as described above, wherein the concentration of gemcitabine in the urine of the individual is between 4 μg/mL and 50 μg/mL during each administration period.

In some embodiments, provided herein is a method of treating high-risk non-muscle invasive bladder cancer (HR-NMIBC) that is unresponsive to intravesical Bacillus Calmette-Guerin (BCG) therapy in an individual comprising (a) administering to the bladder of the individual about 225 mg of gemcitabine during an administration period about every three weeks (Q3W) for at least about 24 weeks, and (b) inducing a complete response in the individual, and wherein each administration period comprises a delivery period of at least seven days.

In some embodiments, provided herein is a method of treating high-risk non-muscle invasive bladder cancer (HR-NMIBC) that is unresponsive to intravesical Bacillus Calmette-Guerin (BCG) therapy in an individual comprising a 24 week induction phase comprising administering about 225 mg gemcitabine to the bladder of the individual about every 3 weeks (Q3W) during an administration period, and an 18 month maintenance phase comprising administering about 225 mg gemcitabine to the bladder of the individual about every twelve weeks during an administration period, wherein each administration period comprises a delivery period of at least seven days when gemcitabine is released from an intravesical drug delivery system, and wherein the intravesical drug delivery system is removed about 3 weeks after each administration. In some embodiments, after each administration period in i) the intravesical drug delivery system is removed and replaced with a new intravesical drug delivery system for a total of about 24 weeks.

In some embodiments, according to any of the methods described above, the gemcitabine administered to the individual is a monotherapy for treatment of the HR-NMIBC that is unresponsive to intravesical BCG therapy.

In some embodiments, according to any of the methods described above, such treatment results in a complete response rate in a population of patients who have received such treatment of at least 50%.

In some embodiments, according to any of the methods described above, the complete response rate in the population of patients is about 60% to about 87%. In some embodiments, the complete response rate in the population of patients is between about 71% to about 91%.

In some embodiments, according to any of the methods described above, the complete response rate in the population of patients is about 87%, about 83%, about 82%, about 79%, about 77%, or about 73%.

In some embodiments, according to any of the methods described above, the complete response rate in the population of patients is improved compared to the standard of care, optionally wherein the complete response rate in the population of patients is improved at least 1.5 fold compared to the complete response rate for standard of care.

In some embodiments, according to any of the methods described above, the complete response rate in the population of patients is improved compared to a checkpoint inhibitor therapy, optionally wherein the complete response rate is improved at least 1.5 fold compared to the complete response rate for the checkpoint inhibitor therapy.

In some embodiments, according to any of the methods described above, the complete response rate in the population of patients is improved compared to treatment with valrubicin, optionally wherein the complete response rate is improved at least 1.5 fold compared to the complete response rate for valrubicin.

In some embodiments, according to any of the methods described above, the individual has a complete response within three months of the start of treatment with gemcitabine.

In some embodiments, provided herein is a method of treating high-risk non-muscle invasive bladder cancer (HR-NMIBC) that is unresponsive to intravesical Bacillus Calmette-Guerin (BCG) therapy in an individual comprising administering to the individual about 360 mg of an antibody that binds to PD-1 about every three weeks (Q3W) for about 6 months, wherein the antibody that binds to PD-1 comprises a variable heavy chain (VH) and a variable light chain (VL), wherein the VH comprises a CDRH1 comprising the amino acid sequence set forth in SEQ ID NO:1, a CDRH2 comprising the amino acid sequence set forth in SEQ ID NO:2, a CDRH3 comprising the amino acid sequence set forth in SEQ ID NO:3; and the VL comprises a CDRL1 comprising the amino acid sequence set forth in SEQ ID NO: 4, a CDRL2 comprising the amino acid sequence set forth in SEQ ID NO: 5, and a CDRL3 comprising the amino acid sequence set forth in SEQ ID NO: 6.

In some embodiments, provided herein is the method of treating high-risk non-muscle invasive bladder cancer (HR-NMIBC) that is unresponsive to intravesical Bacillus Calmette-Guerin (BCG) therapy in an individual as described herein, wherein the complete response rate is at least 30%.

In some embodiments, provided herein is the method of treating high-risk non-muscle invasive bladder cancer (HR-NMIBC) that is unresponsive to intravesical Bacillus Calmette-Guerin (BCG) therapy in an individual as described herein, wherein the complete response rate is about 33%.

In some embodiments, provided herein is the method of treating high-risk non-muscle invasive bladder cancer (HR-NMIBC) that is unresponsive to intravesical Bacillus Calmette-Guerin (BCG) therapy in an individual as described herein, wherein the antibody that binds to PD-1 is administered to the individual as a monotherapy for treatment of the high-risk non-muscle invasive bladder cancer (HR-NMIBC) that is unresponsive to intravesical Bacillus Calmette-Guerin (BCG) therapy.

In some embodiments, provided herein is the method of treating high-risk non-muscle invasive bladder cancer (HR-NMIBC) that is unresponsive to intravesical Bacillus Calmette-Guerin (BCG) therapy in an individual as described herein, wherein the antibody that binds to PD-1 is administered every three weeks (Q3W) for about 18 months.

In some embodiments, according to any of the methods described above, a complete response is determined on the basis of a negative cytology analysis.

In some embodiments, according to any of the methods described above, a complete response is determined on the basis of a negative biopsy.

In some embodiments, according to any of the methods described above, the individual is administered gemcitabine within 12 months of completion of the last dose of BCG therapy.

In some embodiments, according to any of the methods described above, the individual has received i) a minimum of five of six full doses of an induction course of BCG; and ii) two or three doses of a maintenance course, or two of six doses of a second induction course of BCG therapy.

In some embodiments, according to any of the methods described above, the individual has carcinoma in situ (CIS) and/or wherein the population of patients comprises one or more individuals with CIS.

In some embodiments, according to any of the methods described above, the individual has a histologically confirmed diagnosis of persistent or recurrent CIS and/or wherein the population of patients comprises one or more individuals with a histologically confirmed diagnosis of persistent or recurrent CIS.

In some embodiments, according to any of the methods described above, wherein the individual has Ta stage bladder cancer, and/or wherein the population of patients comprises one or more individuals with Ta stage bladder cancer.

In some embodiments, according to any of the methods described above, the individual has high grade Ta stage bladder cancer and/or wherein the population of patients comprises one or more individuals with high grade Ta stage bladder cancer.

In some embodiments, according to any of the methods described above, the individual has T1 stage bladder cancer and/or wherein the population of patients comprises one or more individuals with T1 stage bladder cancer.

In some embodiments, according to any of the methods described above, the individual has high grade T1 stage bladder cancer and/or wherein the population of patients comprises one or more individuals with high grade T1 stage bladder cancer.

In some embodiments, according to any of the methods described above, the individual has papillary bladder cancer and/or wherein the population of patients comprises one or more individuals with papillary bladder cancer.

In some embodiments, according to any of the methods described above, the individual has a mixed histology tumor and/or wherein the population of patients comprises one or more individuals with a mixed histology tumor.

In some embodiments, according to any of the methods described above, the individual is ineligible or elected not to undergo radical cystectomy.

In some embodiments, according to any of the methods described above, the individual elected not to undergo radical cystectomy due to bladder preservation or concern about quality of life after bladder removal.

In some embodiments, according to any of the methods described above, the bladder cancer does not have neuroendocrine, micropapillary, signet ring cell, plasmacytoid, or sarcomatoid features.

In some embodiments, according to any of the methods described above, the individual does not have a urothelial carcinoma or histological variant at any site outside of the urinary bladder.

In some embodiments, according to any of the methods described above, the method comprises performing a transurethral resection of bladder tumor (TURBT) if the individual has papillary bladder cancer.

In some embodiments, according to any of the methods described above, the individual has carcinoma in situ (CIS) following TURBT.

In some embodiments, the individual is renally impaired and/or hepatically impaired.

In some embodiments, the individual is at least 65 years old.

In some embodiments, according to any of the methods described above, wherein following treatment with gemcitabine, the individual is monitored for recurrence.

In some embodiments, according to any of the methods described above, wherein following treatment with gemcitabine, the individual does not receive valrubicin, systemic gemcitabine, docetaxel, pembrolizumab or paclitaxel for treating the high-risk non-muscle invasive bladder cancer (HR-NMIBC) that is unresponsive to intravesical Bacillus Calmette-Guerin (BCG) therapy.

In some embodiments, according to any of the methods described above, the treatment with gemcitabine causes tumor ablation.

In some embodiments, according to any of the methods described above, wherein following treatment with gemcitabine, the individual does not receive a transurethral resection of bladder tumor (TURBT).

In some embodiments, according to any of the methods described above, the individual does not progress to muscle-invasive bladder cancer.

In some embodiments, according to any of the methods described above, the quality-of-life score of the individual is maintained or increases following treatment with gemcitabine.

In some embodiments, according to any of the methods described above, the individual has asymptomatic macrohematuria or dysuria prior to the treatment with gemcitabine.

In some embodiments, according to any of the methods described above, administering gemcitabine comprises inserting an intravesical drug delivery system comprising about 225 mg gemcitabine into the bladder of the individual and removing the intravesical drug delivery system about three weeks later.

In some embodiments, according to any of the methods described above, the method comprises i) inserting an intravesical drug delivery system comprising about 225 mg of gemcitabine into the bladder of the individual; ii) removing the intravesical drug delivery system about three weeks later; iii) inserting a new intravesical drug delivery system comprising about 225 mg of gemcitabine into the bladder on the day that the intravesical drug delivery system is removed in step ii), iv) removing the intravesical drug delivery system about three weeks later; wherein steps ii) and iii) are completed seven times.

In some embodiments, according to any of the methods described above, the method comprises a dosing schedule of at least 24 weeks comprising i) inserting a first intravesical drug delivery system into the bladder in week 0, and removing the first intravesical drug delivery system in week 3; ii) inserting a second intravesical drug delivery system into the bladder in week 3 after removing the first intravesical drug delivery system, and removing the second intravesical drug delivery system in week 6; iii) inserting a third intravesical drug delivery system into the bladder in week 6 after removing the second intravesical drug delivery system, and removing the third intravesical drug delivery system in week 9; iv) inserting a fourth intravesical drug delivery system into the bladder in week 9 after removing the third intravesical drug delivery system, and removing the fourth intravesical drug delivery system in week 12; v) inserting a fifth intravesical drug delivery system into the bladder in week 12 after removing the fourth intravesical drug delivery system, and removing the fifth intravesical drug delivery system in week 15; vi) inserting a sixth intravesical drug delivery system into the bladder in week 15 after removing the fifth intravesical drug delivery system, and removing the sixth intravesical drug delivery system in week 18; vii) inserting a seventh intravesical drug delivery system into the bladder in week 18 after removing the sixth intravesical drug delivery system, and removing the seventh intravesical drug delivery system in week 21; viii) inserting an eighth intravesical drug delivery system into the bladder in week 21 after removing the seventh intravesical drug delivery system, and removing the eighth intravesical drug delivery system in week 24; wherein each intravesical drug delivery system comprises an intravesical device comprising about 225 mg of gemcitabine.

In some embodiments, according to any of the methods described above, the method further comprises a maintenance phase, wherein the maintenance phase comprises administering about 225 mg of gemcitabine to the bladder of the individual for about three weeks about every two to four months.

In some embodiments, according to the method described above, the maintenance phase comprises administering about 225 mg gemcitabine to the bladder of the individual for about three weeks about every three months.

In some embodiments, according to any of the methods described above, the maintenance phase begins at least 30 weeks after the start of treatment with gemcitabine.

In some embodiments, according to any of the methods described above, the maintenance therapy begins about 36 weeks after the start of treatment with gemcitabine.

In some embodiments, according to any of the methods described above, the maintenance therapy comprises administering about 225 mg gemcitabine to the bladder of the individual during weeks 36-39, 48-51, 60-63, 72-75, 84-87, and 96-99.

In some embodiments, according to any of the methods described above, the method comprises i) inserting an intravesical drug delivery system into the bladder of the individual on about week 36 and removing the intravesical drug delivery system on about week 39; ii) inserting an intravesical drug delivery system into the bladder of the individual on about week 48 and removing the intravesical drug delivery system on about week 51; iii) inserting an intravesical drug delivery system into the bladder of the individual on about week 60 and removing the intravesical drug delivery system on about week 63; iv) inserting an intravesical drug delivery system into the bladder of the individual on about week 72 and removing the intravesical drug delivery system on about week 75; v) inserting an intravesical drug delivery system into the bladder of the individual on about week 84 and removing the intravesical drug delivery system on about week 87; vi) inserting an intravesical drug delivery system into the bladder of the individual on about week 96 and removing the intravesical drug delivery system on about week 99; wherein the intravesical drug delivery system comprises an intravesical device comprising about 225 mg of gemcitabine.

In some embodiments, according to any of the methods described above, the method comprises a dosing schedule of at least 99 weeks comprising i) inserting a first intravesical drug delivery system into the bladder in week 0, and removing the first intravesical drug delivery system in week 3; ii) inserting a second intravesical drug delivery system into the bladder in week 3 after removing the first intravesical drug delivery system, and removing the second intravesical drug delivery system in week 6; iii) inserting a third intravesical drug delivery system into the bladder in week 6 after removing the second intravesical drug delivery system, and removing the third intravesical drug delivery system in week 9; iv) inserting a fourth intravesical drug delivery system into the bladder in week 9 after removing the third intravesical drug delivery system, and removing the fourth intravesical drug delivery system in week 12; v) inserting a fifth intravesical drug delivery system into the bladder in week 12 after removing the fourth intravesical drug delivery system, and removing the fifth intravesical drug delivery system in week 15; vi) inserting a sixth intravesical drug delivery system into the bladder in week 15 after removing the fifth intravesical drug delivery system, and removing the sixth intravesical drug delivery system in week 18; vii) inserting a seventh intravesical drug delivery system into the bladder in week 18 after removing the sixth intravesical drug delivery system, and removing the seventh intravesical drug delivery system in week 21; viii) inserting an eighth intravesical drug delivery system into the bladder in week 21 after removing the seventh intravesical drug delivery system, and removing the eighth intravesical drug delivery system in week 24; ix) inserting a ninth intravesical drug delivery system into the bladder in week 36, and removing the ninth intravesical drug delivery system in week 39; x) inserting a tenth intravesical drug delivery system into the bladder in week 48, and removing the tenth intravesical drug delivery system in week 51; xi) inserting an eleventh intravesical drug delivery system into the bladder in week 60, and removing the eleventh intravesical drug delivery system in week 63; xii) inserting a twelfth intravesical drug delivery system into the bladder in week 72, and removing the twelfth intravesical drug delivery system in week 75; xiii) inserting a thirteenth intravesical drug delivery system into the bladder in week 84, and removing the thirteenth intravesical drug delivery system in week 87; xiv) inserting a fourteenth intravesical drug delivery system into the bladder in week 96, and removing the fourteenth intravesical drug delivery system in week 99; wherein each intravesical drug delivery system comprises an intravesical device comprising about 225 mg of gemcitabine.

In some embodiments, according to any of the methods described above, the method comprises i) deploying an intravesical drug delivery system into the bladder once every 3 weeks over approximately 6 months; followed by ii) deploying an intravesical drug delivery system into the bladder once every 12 weeks over approximately 18 months; wherein each intravesical drug delivery system is removed 3 weeks after each administration. In some embodiments, the method comprises deploying a total of about 14 intravesical drug delivery systems over about 2 years.

In some embodiments, according to any of the methods described above, gemcitabine is delivered from the intravesical device during a delivery period.

In some embodiments, according to any of the methods described above, the delivery period is one to three weeks.

In some embodiments, according to any of the methods described above, the concentration of gemcitabine in the urine is from about 1 μg/mL to about 25 μg/mL or from about 4 μg/mL to about 50 μg/mL during the delivery period.

In some embodiments, according to any of the methods described above, the intravesical drug delivery system releases gemcitabine at a rate of 10 mg/day to 45 mg/day for the first seven days of administration.

In some embodiments, according to any of the methods described above, the concentration of gemcitabine in the urine reaches a maximum concentration between about 2 to about 4 days following administration of the gemcitabine.

In some embodiments, according to any of the methods described above, the intravesical drug delivery system releases gemcitabine to produce a mean cumulative excreted gemcitabine in urine value expressed as percentage of the administered dose from 0-168 hours from between about 36% to about 54%. In some embodiments, according to any of the methods described above, the intravesical drug delivery system releases gemcitabine to produce a mean cumulative excreted dFdU amount in urine value expressed as percentage of the administered dose from 0-168 hours from between about 11% to about 23.3%.

In some embodiments, according to any of the methods described above, during each administration period the intravesical drug delivery system releases about 90% of the about 225 mg gemcitabine.

In some embodiments, according to any of the methods described above, during each delivery period the intravesical drug delivery system releases about 70% of the about 225 mg of gemcitabine.

In some embodiments, according to any of the methods described above, the intravesical drug delivery system releases gemcitabine during a first phase of the delivery at a first release rate followed by a second phase of the delivery having a second release rate.

In some embodiments, according to any of the methods described above, the intravesical drug delivery system comprises an intravesical device that comprises a housing which contains and controllably releases the gemcitabine and is elastically deformable between a retention shape configured to retain the intravesical device in the individual's bladder and a deployment shape for passage of the intravesical device through the individual's urethra.

In some embodiments, according to any of the methods described above, the intravesical device comprises a dual lumen silicon part with a single laser-machined orifice.

In some embodiments, according to any of the methods described above, the dual lumen silicon part comprises a large lumen and a small lumen.

In some embodiments, according to any of the methods described above, the small lumen comprises a super-elastic nitinol wire.

In some embodiments, according to any of the methods described above, the super-elastic nitinol wire is in a predefined form to provide retention of the system in the bladder.

In some embodiments, according to any of the methods described above, the large lumen of the dual lumen silicon part comprises the gemcitabine.

In some embodiments, according to any of the methods described above, the large lumen of the dual lumen silicon part comprises osmotic minitablets.

In some embodiments, according to any of the methods described above, the osmotic minitablets contain urea as an osmotic agent.

In some embodiments, according to any of the methods described above, the intravesical drug delivery system is deployed into the bladder of the individual by a urinary placement catheter.

In some embodiments, according to any of the methods described above, the gemcitabine is gemcitabine free base equivalent (FBE).

In some embodiments, according to any of the methods described above, the median duration of response in the population of patients is not reached within about 10.6 months. In some embodiments, the median duration of response in the population of patients is not reached within about 48 weeks.

In some embodiments, such treatment results in a 6-month event-free survival in the population of patients of at least 85%. In some embodiments, the 6-month event-free survival in the population of patients is between about 69% to about 95%. In some embodiments, such treatment results in a 6-month event-free survival in the population of patients of about 87% or about 93%.

In some embodiments, such treatment results in a 12-month event-free survival in the population of patients of at least 70%. In some embodiments, the 12-month event-free survival in the population of patients is between about 50% to about 88%. In some embodiments, such treatment results in a 12-month event-free survival in the population of patients of about 75% or about 84%.

In some embodiments, such treatment results in an 18-month event-free survival in the population of patients of at least 70%. In some embodiments, such treatment results in an 18-month event-free survival in the population of patients between about 50% to about 88%. In some embodiments, such treatment results in an 18-month event-free survival in the population of patients of about 75% or about 84%.

In some embodiments, such treatment results in a 24-month event-free survival in the population of patients of at least 70%. In some embodiments, such treatment results in a 24-month event-free survival in the population of patients between about 50% to about 88%. In some embodiments, such treatment results in a 24-month event-free survival in the population of patients of about 75%.

In some embodiments, provided herein is a kit comprising an intravesical drug delivery system comprising about 225 mg of gemcitabine and instructions for use comprising any of the methods described above.

In some embodiments, provided herein is the kit as described above further comprising a urinary placement catheter.

In some embodiments, according to any of the kits described above, the urinary placement catheter comprises a catheter and a stylet.

In some embodiments, according to any of the kits described above, the kit further comprises a lubricant and/or a syringe.

In some embodiments, provided herein is an article of manufacture comprising an intravesical drug delivery system comprising an intravesical device comprising about 225 mg of gemcitabine and a package insert comprising instructions for use according to any of the methods described above.

In some embodiments, provided herein is an intravesical drug delivery system comprising an intravesical device comprising about 225 mg gemcitabine for use in any of the methods described above.

In some embodiments, provided herein is gemcitabine for use according to any of the methods described above.

In some embodiments, provided herein is use of gemcitabine for treating high-risk non-muscle invasive bladder cancer (HR-NMIBC) that is unresponsive to intravesical Bacillus Calmette-Guerin (BCG) therapy in an individual comprising administering to the individual about 225 mg of gemcitabine about every three weeks (Q3W) for at least about 24 weeks, wherein such treatment results in a complete response rate in a population of patients who have received such treatment of at least 50%.

In some embodiments, provided herein is any of the methods described above, wherein a) less than about 91% of individuals within the population of patients experience an adverse effect; b) less than about 35% of individuals within the population of patients experience a greater than grade 3 adverse effect; c) less than about 40% of individuals within the population of patients experience micturition urgency; d) less than about 40% of individuals within the population of patients experience pollakiuria; e) less than about 30% of individuals within the population of patients experience noninfective cystitis; f) less than about 30% of individuals within the population of patients experience a urinary tract infection; g) less than about 5% of individuals within the population of patients experience pruritus; h) less than about 5% of individuals within the population of patients experience diarrhea; i) less than about 30% of individuals within the population of patients experience dysuria; j) less than about 15% of individuals within the population of patients experience hematuria; k) less than about 10% of individuals within the population of patients experience urinary tract pain; 1) less than about 10% of individuals within the population of patients experience urinary retention; m) less than about 5% of individuals within the population of patients experience renal impairment; and/or n) less than about 5% of individuals within the population of patients experience urosepsis.

In some embodiments, provided herein is minitablets comprising gemcitabine for use according to any of the methods described above.

In some embodiments, provided herein is gemcitabine for use with an intravesical device, for use according to any of the methods described above.

In some embodiments, provided herein is gemcitabine for use according to any of the methods described above, in combination with an intravesical device.

In some embodiments, provided herein is gemcitabine for use according to any of the methods described above, wherein the gemcitabine is administered by an intravesical device.

In some embodiments, the method comprises administering to the individual about 360 mg of an antibody that binds to PD-1 about every three weeks (Q3W) for about 6 months. In some embodiments, the VH comprises the amino acid sequence set forth in SEQ ID NO: 7 and the VL comprises the amino acid sequence set forth in SEQ ID NO:8. In some embodiments, the antibody that binds to PD-1 comprises a heavy chain comprising the amino acid sequence set forth in SEQ ID NO:9 and the light chain comprising the amino acid sequence set forth in SEQ ID NO:10. In some embodiments, the antibody that binds to PD-1 is cetrelimab. In some embodiments, the antibody that binds to PD-1 is administered intravenously.

In some embodiments, according to any of the methods described above, amount of gemcitabine administered to the individual is a clinically effective amount of gemcitabine.

In some embodiments, according to any of the methods described above, there are at least 20 individuals in the population of patients. In some embodiments, according to any of the methods described above, there are 23 individuals in the population of patients. In some embodiments, there are 54 individuals in the population of patients. In some embodiment, there are 85 individuals in the population of patients.

In some embodiments, the individual is ineligible for radical cystectomy due to age, high American Society of Anesthesiologists class score, or medical and surgical comorbidities.

In some embodiments, the individual elects not to undergo a radical cystectomy to preserve their bladder, to preserve sexual function, concern about quality of life after cystectomy, or concern about mortality and morbidity risk associated with radical cystectomy.

In some embodiments, the individual is elderly and/or frail.

In some embodiments, about 51% of patients in the population of patients refuse radical cystectomy to preserve their blader.

In some embodiments, about 37% of patients in the population of patients refuse radical cystectomy because of morbidity and mortality concerns.

In some embodiments, the intravesical drug delivery system is a TAR-200 intravesical drug delivery system.

In some embodiments, the median duration of response in the population of patients is about 26 months.

In some embodiments, such treatment results in a one-year duration of response (DOR) rate in the population of patients of at least 50%. In some embodiments, the one-year duration of response (DOR) rate in the population of patients is between about 50% to about 89%. In some embodiments, the one-year DOR rate is about 77%.

In some embodiments, the individual is at risk of disease progression.

In some embodiments, the individual has an ECOG performance status of 0-2.

In some embodiments, such treatment results in a 12-month CR rate in the population of patients of at least 40%. In some embodiments, the 12-month CR rate in the population of patients is between about 43% to about 79%. In some embodiments, the 12-month CR rate in the population of patients is about 64%.

In some embodiments, such treatment results in a 15-month CR rate in the population of patients of at least 40%. In some embodiments, the 15-month CR rate in the population of patients is between about 43% to about 79%. In some embodiments, the 15-month CR rate in the population of patients is about 64%.

### BRIEF DESCRIPTION OF THE DRAWINGS

The drawings illustrate certain features and advantages of this disclosure. These embodiments are not intended to limit the scope of the appended claims in any manner.

FIGS. 1A-1B show an exemplary intravesical drug delivery system, TAR-200. TAR-200 is an intravesical drug delivery system comprising two components: (i) the drug constituent, which consists of gemcitabine minitablets (225 mg, free base equivalent) and osmotic minitablets containing urea as the osmotic agent; and (ii) the intravesical device constituent, which is comprised of a dual lumen silicone part with a single laser-machined orifice and a superelastic nitinol wire (“wireform”). The large lumen of the silicone part contains the gemcitabine and urea minitablets and serves as an elementary osmotic pump to release drug in a controlled manner. The smaller lumen contains the nitinol wire in a predefined form to provide retention of the system in the bladder during the indwelling period. In FIG. 1A, TAR-200 is shown in an exemplary retention shape (e.g., in a so-called “pretzel shape” or a “bi-oval shape”) suited to retain the intravesical device within the bladder. The retention shape provides, among other things, that the intravesical drug delivery system resists becoming entrained in urine and excreted when the individual voids. In FIG. 1B, a close-up view is shown to highlight the drug constituent and wireform of TAR-200.

FIGS. 2A-2B show exemplary insertion devices for use with intravesical drug delivery systems provided herein, such as TAR-200, called a urological placement catheter (Urinary Placement Catheter (UPC) or Inserter) for the transurethral placement of the intravesical drug delivery systems. The UPC comprises a catheter and stylet, as shown in FIG. 2A and FIG. 2B. In FIG. 2B, a close-up view is shown to highlight the depth markings present on the catheter shaft.

FIGS. 3A-3B show the study design for an open-label, parallel-group, multi-center study of the efficacy and safety of intravesical TAR-200 or systemic cetrelimab in participants with high-risk NMIBC (CIS with or without papillary disease) unresponsive to prior intravesical Bacillus Calmette-Guerin (BCG) therapy who are either ineligible for or have elected not to undergo radical cystectomy (RC). Dosing regimens for Year 1 are shown in FIG. 3A, and dosing regimens for Years 2 and 3 are shown in FIG. 3B.

FIGS. 4A-4C depict swim lane plots showing response events over time for intravesical TAR-200 or systemic cetrelimab treatment in participants with high-risk NMIBC (CIS with or without papillary disease) unresponsive to prior intravesical Bacillus Calmette-Guerin (BCG) therapy who are either ineligible for or have elected not to undergo radical cystectomy (RC). Participants on treatment are indicated with a light grey bar. Results are shown for three timepoints for intravesical TAR-200, a clinical cutoff date of Feb. 20, 2023 (FIG. 4A) and a clinical cutoff date of Apr. 4, 2023 (FIG. 4B), and a clinical cutoff date of Aug. 24, 2023 (FIG. 4C). Results are shown for two timepoints for systemic cetrelimab treatment, a clinical cutoff date of Feb. 20, 2023 (FIG. 4A) and a clinical cutoff date of Apr. 4, 2023 (FIG. 4B). Complete response (CR) is indicated with a dark grey bar, and a non-CR event is indicated with a black square. Duration of response (DOR), where applicable, is indicated to the right of each lane.

FIG. 5 shows a graphical representation of the reasons for refusal of and ineligibility for radical cystectomy (RC).

FIG. 6 shows a graphical breakdown of the reasons for refusal of cystectomy by age, gender, and nicotine status.

FIG. 7 depicts a swim lane plot showing response events over time for intravesical TAR-200 treatment in participants with high-risk NMIBC (CIS with or without papillary disease) unresponsive to prior intravesical Bacillus Calmette-Guerin (BCG) therapy who are either ineligible for or have elected not to undergo radical cystectomy (RC) treated through Jan. 2, 2024 (clinical cut-off date). Participants on treatment are indicated with a light grey bar. Complete response (CR) is indicated with a dark grey bar, and a non-CR event is indicated with a black square. Duration of response (DOR), where applicable, is indicated to the right of each lane.

FIG. 8 depicts the observed complete response (CR) rate for intravesical TAR-200 treatment in participants with high-risk NMIBC (CIS with or without papillary disease) unresponsive to prior intravesical Bacillus Calmette-Guerin (BCG) therapy who are either ineligible for or have elected not to undergo radical cystectomy (RC) as the number of patients in the study expanded from 30 to 58. The 95% confidence interval (CI) is shown in brackets.

FIGS. 9A and 9B depict the observed CR rate across patient subgroups for intravesical TAR-200 treatment in participants with high-risk NMIBC (CIS with or without papillary disease) unresponsive to prior intravesical Bacillus Calmette-Guerin (BCG) therapy who are either ineligible for or have elected not to undergo radical cystectomy (RC). The 95% confidence interval (CI) is shown in brackets. EMEA (Europe, Middle East, and Africa), CIS (carcinoma in situ).

FIGS. 10A-10B depict the duration of response rate over time (FIG. 10A) and the event-free survival rate over time (FIG. 10B) for intravesical TAR-200 treatment in participants with high-risk NMIBC (CIS with or without papillary disease) unresponsive to prior intravesical Bacillus Calmette-Guerin (BCG) therapy who are either ineligible for or have elected not to undergo radical cystectomy (RC).

FIG. 11A and FIG. 11B depict mean (SD) urine concentration of gemcitabine after intravesical administration of TAR-200 (225 mg of Gemcitabine) in two groups of individuals treated with TAR-200. FIG. 11A shows results from week 0. FIG. 11B shows results from week 3, week 6, or week 9 (individuals provided samples for one of these weeks).

FIG. 12A and FIG. 12B depict mean (SD) Gemcitabine Related Components (GRC) in mg-equivalents gemcitabine which are calculated as follows: GRC (mg-Eq. gemcitabine)=Gemcitabine in mg+dFdU in mg* (263.20/264.18) in two groups of individuals treated with TAR-200. FIG. 12A shows results from week 0. FIG. 12B shows results from week 3, week 6, or week 9 (individuals provided samples for one of these weeks).

### DETAILED DESCRIPTION

Prior to the present invention, few therapies were available to treat individuals with HR-NMIBC that were unresponsive to BCG therapy, and the available therapies were relatively ineffective. Individuals with high-risk non-muscle invasive bladder cancer are typically initially treated with BCG, however up to 50% of patients eventually experience recurrence. A subset of patients may receive a radical cystectomy, however the utility of radical cystectomy is limited due to high morbidity and decreased quality of life. As demonstrated herein, administering gemcitabine locally and continuously to the bladder of an individual with high-risk non-muscle invasive bladder cancer offers a significant improvement in clinical response over other existing standard of care treatments. This dramatically increased response allows for the first time, a suitable alternative to radical cystectomy. In some aspects, the present therapy is significantly more effective than the currently available treatment for such patients, such as valrubicin and Keytruda. For example, valrubicin, which was approved more than 20 years ago provides a complete response rate of patients of about 20%. Keytruda (pembrolizumab), an immune checkpoint therapy provides an initial complete response rate in these patients of about 42%, which drops substantially over time. Moreover, the 12-month complete response rate for Keytruda is only 19% (Balar A J, et al. Lancet Oncol. 2021; 22:919-930). The 12-month complete response rates for other treatment options for BCG-unresponsive HR NMIBC CIS such as atezolizumab and nadofaragene firadenovec are only 15% and 23% respectively (Black P C, et al. Eur Urol. 2023; 84:536-544, ADSTILADRIN® (nadofaragene firadenovec-vncg) [prescribing information]). In significant contrast, local and continuous delivery of gemcitabine to the bladder over a period of time to individuals with HR-NMIBC unresponsive to BCG therapy, is now shown to produce a complete response rate in a population of patients receiving such treatment of over 50%. In some aspects, the complete response rate is about 60% to about 80%, or about 73% to about 78%. In some aspects, the complete response rate is 60% to 80%, or 73% to 78%. This significantly improved complete response rate allows for local and continuous delivery of gemcitabine, according to the methods provided herein to provide for the first time a viable alternative to radical cystectomy for these high-risk patients. Accordingly, in some aspects, provided herein is a method of bladder sparing in an individual having high-risk non-muscle invasive bladder cancer. Additionally, individuals with HR-NMIBC that are unresponsive to BCG therapy are at a high risk of disease progression. Consequently, there exists a significant need for therapies to HR-NMIBC that provide rapid response rates. Prompt assessment of these therapies can guide the decision on whether to continue the current treatment or consider alternatives. Significantly, 47 out of 48 individuals treated with the present therapy responded by 12 weeks. Moreover, this response was durable, with a complete response rate at 12 months of 64%, far superior to the 12-month CR rate of 18% observed with pembrolizumab therapy.

In another aspect, the gemcitabine is administered as a monotherapy for treatment of HR-NMIBC that is unresponsive to BCG therapy. Prior to the present invention, it was considered that local delivery of gemcitabine to the bladder may not be adequately effective for treating HR-NMIBC that is unresponsive to BCG therapy, and therefore local delivery of gemcitabine to the bladder should be combined with other therapies, such as checkpoint therapies, in order to generate a sufficient immune response to cause an anti-tumor response. Surprisingly, local and continuous administration of gemcitabine to the bladder as a monotherapy was nearly two times as effective as the existing standard of care.

In further aspects, upon treatment with local and continuous administration of gemcitabine to the bladder, a response is observed at three months, or within about three months of the start of treatment with gemcitabine. In some aspects, individuals who respond to such treatment have a low relapse rate, and/or do not relapse within one month, two months, three months, four months, five months, six months, or one year. In some embodiments, following treatment with gemcitabine the individual does not relapse and therefore does not require a further therapy for treatment of bladder cancer.

The unexpected improvement in complete response rate disclosed herein provides the added benefit of avoiding invasive alternative treatments such as surgical removal of the bladder (radical cystectomy), which results in significant impacts to an individual's quality of life. Such quality-of-life impacts include incontinence, sexual disfunction, infertility, and bowel function complications. Moreover, local administration of gemcitabine results in reduced side effects as compared to systemic chemotherapy. Together, these clinical improvements yield a safe and remarkably effective treatment method for individuals with high-risk non-muscle invasive bladder.

Accordingly, provided herein, in some aspects, are methods of treating individuals with high-risk non-muscle invasive bladder cancer by administering gemcitabine locally to the bladder of the individual, wherein each gemcitabine administration comprises delivering gemcitabine continuously to the bladder for at least 7 days during a gemcitabine delivery period. In some embodiments, gemcitabine is administered to the bladder about every three weeks for up to about the first six months and administered about every three months thereafter. In some embodiments, gemcitabine is administered to the bladder for about six months with no rest periods. In some embodiments, gemcitabine is administered to the bladder about every three weeks for the first 24 weeks and then about every three months thereafter for a total of about 2 years. In some embodiments, gemcitabine is administered to the bladder for about 24 weeks without a rest period and then is administered for three weeks at a time with a three month rest period between administrations. In some embodiments, about 225 mg of gemcitabine is administered during each administration. In some embodiments, the individual has BCG unresponsive non-muscle invasive bladder cancer. In some embodiments, the individual has refused or is ineligible for radical cystectomy.

In another aspect, provided herein, are methods of treating individuals with high-risk non-muscle invasive bladder cancer comprising administering to the individual an antibody that binds to PD-1. Also provided is an antibody that binds to PD-1 for use in methods of treating individuals with high-risk non-muscle invasive bladder cancer comprising administering to the individual an antibody that binds to PD-1. In some embodiments, the antibody that binds to PD-1 is administered about every three weeks for 18 months. In some embodiments, about 360 mg of the PD-1 antibody is administered (e.g., via intravenous infusion) during each administration. In some embodiments, the individual has BCG unresponsive non-muscle invasive bladder cancer. In some embodiments, the individual has refused or is ineligible for radical cystectomy.

### Definitions

Reference to “about” a value or parameter herein includes (and describes) variations that are directed to that value or parameter per se. For example, description referring to “about X” includes description of “X”.

“Administration” or “administering” as used herein refers to providing a therapeutic agent to an individual. Administration can be subcutaneous, intravenous, or intravesical, for example. In some embodiments, a particular amount of drug is administered to the individual, however only a portion of the drug is delivered (or released). In certain embodiments, an administration period is a period during which an intravesical drug delivery system comprising a drug component, such as gemcitabine, remains in the individual's bladder.

“Delivery” is used herein to refer to release of a drug. For example, a delivery period is a period during which an effective amount or clinically effective amount, of a drug, such as gemcitabine, is released from, for example, an intravesical drug delivery system.

As used herein, the singular forms “a,” “an,” and “the” include plural referents unless the context clearly dictates otherwise. For example, “a” or “an” means “at least one” or “one or more.”

The term “continuous” or “continuously” as used herein refers to sustained administration of a therapeutic agent (such as gemcitabine) over a sustained period of time. Continuous includes different release rates over a period of time. For example, a drug is continuously released over a period of seven days if the drug is released at a faster rate over the first three days and a slower rate over a period of the last four days.

The term “individual” as used herein refers to a human.

Reference to “about” a value or parameter herein includes (and describes) embodiments that are directed to that value or parameter per se. For example, “about three weeks” includes 17, 18, 19, 20, 21, 22, 23, 24, and 25 days.

The term “about X-Y” used herein has the same meaning as “about X to about Y.”

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), suppressing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer. The methods of the invention contemplate any one or more of these aspects of treatment.

The term ““methods of treatment” used herein is intended to be synonymous with “medical use”. For example, disclosure of “a method of treating HR-NMIBC comprising administering gemcitabine to a patient” is equivalently to and includes “use of gemcitabine for treating HR-NMIBC in a patient”.

The term “effective amount” used herein refers to an amount of a compound or composition sufficient to treat a specified disorder, condition or disease such as to ameliorate, palliate, lessen, and/or delay one or more of its symptoms. In reference to cancers, an effective amount comprises an amount sufficient to cause a tumor to shrink and/or to decrease the growth rate of the tumor (such as to suppress tumor growth) and/or to suppress, or delay other unwanted cell proliferation and/or to treat or suppress tumor metastasis and/or to reduce (such as eradiate) preexisting tumor metastasis and/or to reduce incidence or burden of preexisting tumor metastasis and/or to suppress or delay tumor metastasis and/or to inhibit tumor cells and/or to induce an immune response against a tumor cell. An effective amount can be administered in one or more administrations, for example, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) suppress or delay occurrence and/or recurrence of tumor; and/or (vii) relieve to some extent one or more of the symptoms associated with the cancer.

The term “month” used herein refers to about 28 days to about 31 days.

“BCG-unresponsive” high-risk NIMBC as used herein means that the individual has at least one of the following: persistent disease despite adequate BCG therapy; disease recurrence after initially achieving a tumor-free state after adequate BCG; or T1 tumors following a single induction course of BCG.

“Adequate BGC therapy” as used herein means a) at least 5 of 6 doses of an induction course plus b) at least 2 of 3 doses or a maintenance course or at least 2 of 6 doses of a second induction course.

“Monotherapy” as used herein means use of a treatment regimen, such as treatment comprising local administration of gemcitabine to the bladder, as the single active ingredient for treating HR-NMIBC. In some embodiments, the individual receiving monotherapy receives one or more therapy to treat a disease other than HR-NMIBC or a side effect of the monotherapy. Gemcitabine may be provided in the form of a pharmaceutically acceptable salt. Amounts of gemcitabine are on free base basis, unless indicated otherwise.

### I. Methods of Treatment

The various aspects and embodiments described in this section in the context of a method of treatment also apply to gemcitabine for use according to the methods described herein, and minitablets comprising gemcitabine for use according to the methods described herein, unless indicated otherwise. Similarly, the various aspects and embodiments described in this section in the context of a method of treatment also apply to gemcitabine for use with an intravesical drug delivery system, according to the methods described herein, and gemcitabine for use according to the methods described herein, in combination with an intravesical drug delivery system, and gemcitabine for use according to the methods described herein, wherein the gemcitabine is administered by an intravesical drug delivery system.

### A. Patient Populations

The present invention in some aspects provides a method of treating HR-NMIBC bladder cancer that is unresponsive to BCG in an individual. In some embodiments, the individual has a histologically variant subtype of a urothelial carcinoma of the lower tract, such as papillary, micropapillary, or carcinoma in situ. In some embodiments, the carcinoma in situ is lobular carcinoma in situ (LCIS) or ductal carcinoma in situ. In some embodiments, the individual is at risk of disease progression.

In some embodiments, the individual is unfit or not eligible for a cystectomy or has refused a cystectomy. In some embodiments, the individual is ineligible for radical cystectomy under the National Comprehensive Cancer Network (NCCN) guidelines. For example, the individual is unfit for curative therapy due to frailty. Prior to the present methods, such individuals typically received palliative radiation without chemotherapy (3.5 Gy/fraction—10 treatments; or 7 Gy/fraction—7 treatments; TURBT; or no treatment). In some embodiments, the individual cannot tolerate radical cystectomy based upon the American Society of Anesthesiology (ASA) guidelines. For example, the individual who cannot tolerate radial cystectomy may be deemed medically unfit for surgery requiring general or epidural anesthesia.

In other embodiments, the individual may not be suitable for radical cystectomy due to a lack of post-operative care infrastructure (e.g., as determined by the Comprehensive Geriatric Assessment provided by the American Society of Anesthesiologists). In some embodiments, an individual is not suitable for radical cystectomy due to frailty (e.g., as determined by the Comprehensive Geriatric Assessment provided by the American Society of Anesthesiologists). Under these guidelines, an individual is deemed frail if he or she shows abnormal independent activities of daily living, severe malnutrition, cognitive impairment, or comorbidities cumulative illness rating scale for geriatrics (CISR-G) grades 3-4. Furthermore, under current guidelines, subjects must be deemed unfit for radical cystectomy (RC) due to comorbid conditions with a risk of mortality ≥5% as estimated by the American College of Surgeons risk calculator using the current procedure terminology code 51595 or 51596 for cystectomy.

In some embodiments, the individual is eligible for a radical cystectomy but elects not to undergo the radical cystectomy due to quality-of-life considerations. In some embodiments, the quality-of-life impacts of radical cystectomy include, but are not limited to, mortality, incontinence, decreased sexual function, subfertility or infertility, and decreased bowel function. In some embodiments, more than about 40% of patients in a population of patients refuse radical cystectomy to preserve their bladder. In some embodiments, between about 45% to about 55% of patients in a population of patients refuse radical cystectomy to preserve their bladder. In some embodiments, at least about 45% of patients in a population of patients refuse radical cystectomy to preserve their bladder. In some embodiments, about 51% of patients in a population of patients refuse radical cystectomy to preserve their bladder. In some embodiments, more than about 30% of patients in a population of patients refuse radical cystectomy because of morbidity and mortality concerns. In some embodiments, between about 30% to about 45% of patients in a population of patients refuse radical cystectomy because of morbidity and mortality concerns. In some embodiments, at least about 35% of patients in a population of patients refuse radical cystectomy because of morbidity and mortality concerns. In some embodiments, about 37% of patients in a population of patients refuse radical cystectomy because of morbidity and mortality concerns.

In some embodiments, the individual is assessed based on an ECOG Performance Status Scale. In some embodiments, the ECOG performance scale measures an individual's level of functioning in terms of their ability to care for themselves, daily activity, and physical ability. In some embodiments, an individual with an ECOG performance status of 0 is fully active, and able to carry on all pre-disease performance without restriction. In some embodiments, an individual with an ECOG performance status of 1 is restricted in physically strenuous activity but ambulatory and able to carry out work of a light or sedentary nature. In some embodiments, an individual with an ECOG performance status of 2 is ambulatory and capable of all selfcare but unable to carry out any work activities. In some embodiments, the individual has an ECOG performance status of 0-2. In some embodiments, the individual has an ECOG performance status of 0. In some embodiments, the individual has an ECOG performance status of 1. In some embodiments, the individual has an ECOG performance status of 2.

In some aspects, the individual has non-muscle invasive bladder cancer (NMIBC). In some embodiments, NMIBC can be risk stratified into low, intermediate and high-risk groups depending on the probability of recurrence and progression to muscle-invasive disease. In some embodiments, the individual has high-risk NMIBC (HR-NMIBC). In some embodiments, the individual has high-risk NMIBC (CIS with or without papillary disease) unresponsive to prior intravesical Bacillus Calmette-Guerin (BCG) therapy. In some embodiments, the individual is either ineligible for or has elected not to undergo radical cystectomy (RC). In some embodiments, the individual is diagnosed with high-risk, non-muscle invasive urothelial carcinoma in situ (CIS; Tis), with or without papillary disease (T1, high-grade Ta) within 12 months of completion of adequate BCG therapy. In some embodiments, the individual has BCG-unresponsive high-risk NMIBC after treatment with adequate BCG therapy defined as a minimum of 5 of 6 doses of an induction course (adequate induction) plus 2 of 3 doses of a maintenance course, or 2 of 6 doses of a second induction course. In some embodiments, the individual is eligible for a radical cystectomy.

In some embodiments, the NMIBC is staged using the tumor, node, metastasis (TNM) staging system. In some embodiments, Ta stage bladder cancer is defined as a non-invasive papillary carcinoma. In some embodiments, Ta stage bladder cancer has grown toward the hollow center of the bladder but has not grown into the connective tissue or muscle of the bladder wall. In some embodiments, Ta stage bladder cancer is further delineated into either low-grade (LG) or high-grade (HG) subtypes, with LG referring to a slow growing, less aggressive form of the disease and HG referring to a rapidly growing, more aggressive form of the disease. In some embodiments, CIS is defined as a flat lesion comprising of cytologically malignant cells which may involve either full or partial thickness of the urothelium. In some embodiments, the NMIBC is identified by clinical staging (cTa, cT1) based on endoscopic surgery (biopsy or TURBT).

In some embodiments, the individual has undergone prior BCG therapy. In some embodiments, the prior BCG treatment comprises intravesical instillations of hundreds of millions of Mycobacterium bovis Bacillus Calmette-Calmette-Guérin bacilli applied weekly over the course of a six week induction treatment. In some embodiments, the bladder cancer is resected following the six-week BCG induction treatment. In some embodiments, the prior BCG therapy further comprises a maintenance treatment consisting of six-week periods of intravesical BCG instillation every three months for one to three years.

In some embodiments, the individual has undergone between 3 and 6 courses of BCG induction treatment. In some embodiments, the individual has undergone at least 1 to 2, 2 to 3, 3 to 4, 4 to 5, or 5 to 6 prior courses of BCG induction treatment. In some embodiments, the individual has undergone 5 courses of BCG induction treatment during the prior BCG therapy. In some embodiments, the individual has undergone at least 7, 8, 9, 10, 11, 12, 13, 14, 15, or 16 prior courses of BCG therapy. In some embodiments, adequate BCG therapy is defined as a minimum of 5 of 6 full doses of an induction course plus 2 of 3 doses of a maintenance course, or at least 2 of 6 doses of a second induction course. In some embodiments, individuals receiving BCG therapy receive screening endoscopic surgeries (biopsies or TURBTs) about every 3 months to monitor for disease progression.

In some embodiments, BCG-unresponsive HR-NMIBC is defined as persistent disease despite adequate BCG therapy, disease recurrence after initially achieving a tumor-free state after adequate BCG, or T1 tumors following a single induction course of BCG. In some embodiments, an individual becomes aware of BCG-unresponsive HR-NMIBC after experiencing painful urination (dysuria). In some embodiments, an individual becomes aware of BCG-unresponsive HR-NMIBC after experiencing blood in the urine without pain (asymptomatic microhematuria). In some embodiments, BCG unresponsive disease is defined as recurrent high-grade Ta or any T1 disease within 6 months of completion of adequate BCG therapy, or T1 high-grade disease at the first disease assessment following an induction BCG course.

In some embodiments, the bladder cancer is resected prior to administration of the gemcitabine. In some embodiments, the individual undergoes a TURBT prior to administration of the gemcitabine to the bladder. In some embodiments, gemcitabine is administered within seven days following a TURBT. In some embodiments, the tumor is maximally resected prior to administration of the gemcitabine such that no visible tumor is present. In some embodiments, the tumor is non-maximally resected prior to administration of the gemcitabine. In some embodiments, the tumor is non-maximally resected prior to administration of the gemcitabine such that residual tumor is present. In some embodiments, the patient is TO after TURBT. In some embodiments, the individual undergoes TURBT at week 24 and/or week 48 of treatment with gemcitabine.

In some embodiments, the individual has undergone TURBT and has residual tumor at the site of resection. In some embodiments, the individual has stage Ta, or Tis cancer following TURBT.

In some embodiments, the individual does not have lymph node involvement (N+) and/or metastases (M+) per Blinded Independent Central Review of CT/MR Urography. In some embodiments, the individual does not have active malignancies other than the disease being treated under study. In some embodiments, active malignancies are defined as malignancies progressing or requiring treatment change in the last 24 months.

In some embodiments, the individual is renally impaired and/or hepatically impaired. In some embodiments, the individual is renally impaired. In some embodiments, renal impairment is measured using a creatine clearance rate calculated using the Cockcroft-Gault formula. In some embodiments, the individual is hepatically impaired. In some embodiments, hepatic function is measured with total bilirubin, alanine aminotransferase (ALT), or aspartate aminotransferase (AST) levels.

In some embodiments, the individual is elderly. In some embodiments, the individual is at least 65 years old. In some embodiments, the individual is between about 65 to about 70, about 70 to about 75, about 75 to about 80, about 80 to about 85, or about 85 to about 90 years old. In some embodiments, the individual is more than 65 years old. In some embodiments, the individual is frail.

### B. Dosing Regimens

The following section describes various aspects (embodiments) of dosing and treatment regimens, any and all of which apply to the methods described herein. The various aspects (embodiments) of dosing and treatment regimens also apply to gemcitabine for use according to the methods described herein, and minitablets comprising gemcitabine for use according to the methods described herein. Similarly, the various aspects (embodiments) of dosing and treatment regimens also apply to gemcitabine for use with an intravesical drug delivery system, according to the methods described herein, and gemcitabine for use according to the methods described herein, in combination with an intravesical drug delivery system, and gemcitabine for use according to the methods described herein, wherein the gemcitabine is administered by an intravesical drug delivery system.

The present application in one aspect provides a method of treating bladder cancer in an individual comprising administering gemcitabine locally to the bladder of the individual, wherein each gemcitabine administration comprises delivering gemcitabine continuously to the bladder for at least 7 days during a gemcitabine delivery period. In some embodiments, gemcitabine is administered as a monotherapy.

In some embodiments, the gemcitabine is administered by inserting an intravesical drug delivery system into the bladder of the individual, wherein the administration period is the time that the device is maintained in the bladder. In some embodiments, the gemcitabine is delivered from the device into the urine in the bladder during a delivery period that is less than the entire administration period. For example, in some embodiments, the methods provided herein comprise a three week administration period during which the intravesical drug delivery system resides in the bladder, wherein the intravesical drug delivery system releases gemcitabine for at least one week, but less than three weeks.

In some aspects, provided herein is a method of treating a bladder cancer in an individual comprising administering gemcitabine locally to the bladder of the individual about every 3 weeks, wherein each gemcitabine administration comprises delivering gemcitabine continuously to the bladder for at least 7 days during a gemcitabine delivery period. In some embodiments, about 225 mg of gemcitabine is administered. In some embodiments, administering gemcitabine comprises inserting an intravesical drug delivery system comprising about 150 mg to about 300 mg, about 200 mg to about 250 mg, or about 225 mg of gemcitabine into the bladder and removing the intravesical drug delivery system about three weeks later.

In some embodiments, the delivery period comprises local and continuous delivery of gemcitabine to the bladder. In some embodiments, the concentration of the gemcitabine or active metabolite thereof in the plasma of the individual is at a subtherapeutic level during portions of the delivery period.

In some embodiments, gemcitabine is administered to the individual about every three weeks. In some embodiments, administration of gemcitabine comprises inserting an intravesical drug delivery system and removing the intravesical drug delivery system about three weeks later. In some embodiments, the intravesical system delivers gemcitabine during a gemcitabine delivery period which comprises at least a portion of the about three week administration period. In some embodiments, the gemcitabine delivery period is at least one week. In some embodiments, the gemcitabine delivery period is at least two weeks. In some embodiments, the gemcitabine delivery period is at least about three weeks. In some embodiments, the gemcitabine delivery period is at least about two to about three weeks. In some embodiments, the gemcitabine delivery period is about one week to about three weeks. In some embodiments, the length of the gemcitabine delivery period is the same or about the same for each delivery period. In some embodiments, there is an effective concentration of gemcitabine in the urine of the individual during the gemcitabine delivery period.

In some of embodiments, each delivery period is about three weeks. In some embodiments, each delivery period is about 14 to about 21 days. In some embodiments, the gemcitabine is delivered multiple times over a period of more than six weeks. In some embodiments, the gemcitabine is delivered multiple times over a period of more than 12 weeks. In some embodiments, gemcitabine is delivered multiple times over a period of more than 15 weeks. In some embodiments, gemcitabine is delivered multiple times over a period of more than 18 weeks. In some embodiments, the gemcitabine is delivered multiple times over a period of more than 21 weeks. In some embodiments, gemcitabine is delivered multiple times over a period of about 24 weeks.

In some embodiments, the gemcitabine is continuously delivered to the bladder for about 7, 8, 9, 10, 11, 12, 13 or 14 days. In some embodiments, the gemcitabine is continuously delivered to the bladder for up to 21 days (e.g., for about 15, 16, 17, 18, 19, 20, or 21 days). In some embodiments, the majority of the gemcitabine is delivered to the bladder within the first 14 days following insertion of the intravesical drug delivery system.

It may be advantageous to continuously deliver gemcitabine locally to the bladder more than once. For example, in some embodiments, the gemcitabine is delivered locally to the bladder of an individual at least twice, at least 3 times, at least 4 times, at least 5 times, or at least 10 times. In some embodiments, gemcitabine is delivered multiple times over a period of at least 12 weeks, at least 15 weeks, at least 18 weeks, at least 21 weeks, at least 24 weeks, at least 27 weeks, or at least 30 weeks. In some embodiments, gemcitabine is delivered multiple times over a period of at least 1 month, at least 2 months, at least 3 months, at least 4 months, at least 5 months, at least 6 months, at least 12 months, or at least 18 months. For example, in some embodiments, gemcitabine is delivered locally to the bladder of an individual twice, 3 times, 4 times, 5 times, or 10 times. In some embodiments, gemcitabine is delivered multiple times over a period of one month, one month to 18 months, 2 months to 18 months, 3 months to 18 months, or one month to 6 months.

In some embodiments, during the gemcitabine delivery period, there is an effective amount of gemcitabine in the urine of the individual for treating a bladder cancer. In some embodiments, the urine concentration of gemcitabine during the delivery period is about 1 μg/mL to about 25 μg/mL, about 1 μg/mL to about 20 μg/mL, about 1 μg/mL to about 15 μg/mL, about 1 μg/mL to about 10 μg/mL. In some embodiments, the urine concentration of gemcitabine during the delivery period is about 18.4 μg/mL. In some embodiments, the maximum urine concentration of gemcitabine during the delivery period is about 18.4 μg/mL. In some embodiments, the urine concentration of gemcitabine during the delivery period is about 16.1 μg/mL. In some embodiments, the urine concentration of gemcitabine during the delivery period is about 8.9 μg/mL. In some embodiments, the urine concentration of gemcitabine during the delivery period is about 6.9 μg/mL. In some embodiments, the urine concentration is at least 4-5 μg/mL for at least one week. In some embodiments, the urine concentration peaks on day 2 following insertion of an intravesical drug delivery system into the bladder of the individual. In some embodiments, the urine concentration of gemcitabine ranges between about 4 μg/mL to about 40 μg/mL over the delivery period. In some embodiments, the urine concentration of gemcitabine ranges between about 4 μg/mL to about 40 μg/mL over about seven days. In some embodiments, the delivery period is less than three weeks. In some embodiments, the delivery period is one to three weeks. In some embodiments, the delivery period is one to two weeks. In some embodiments, administering gemcitabine comprises inserting an intravesical drug delivery system comprising gemcitabine into the bladder of the individual and removing the intravesical drug delivery system about three weeks later. In some embodiments, the concentration of the gemcitabine or active metabolite thereof in the plasma of the individual is at a subtherapeutic level during the delivery period. In some embodiments, upon delivery of gemcitabine the ratio of gemcitabine in the urine to gemcitabine in the plasma of the individual during the period of continuous delivery is greater than about 500:1. In some embodiments, the main metabolite of gemcitabine is 2′,2′ difluorodeoxyuridine (dFdU). In some embodiments, the plasma concentration of dFdU is less than 0.3 μg/mL upon delivery of the gemcitabine. In some embodiments, the plasma concentration of dFdU is less than 0.2 μg/mL upon delivery of the gemcitabine. In some embodiments, the plasma concentration of dFdU is less than 0.1 μg/mL upon delivery of the gemcitabine. In some embodiments, the plasma concentration of dFdU is between 0.1 and 0.3 μg/mL upon delivery of the gemcitabine.

In some embodiments, the method comprises administering gemcitabine locally to the bladder of the individual every three weeks or about every three weeks during an induction phase. In some embodiments, the dosing regimen comprises a maintenance phase following the induction phase. In some embodiments, the maintenance phase comprises administering an effective amount of gemcitabine locally to the bladder of the individual about every three months following the induction phase. In some embodiments, about 225 mg of gemcitabine is administered during each about three week period. In some embodiments, administering gemcitabine comprises inserting an intravesical drug delivery system comprising about 150 mg to about 300 mg, about 200 mg to about 250 mg, or about 225 mg of gemcitabine into the bladder and removing the intravesical drug delivery system about three weeks later.

In some embodiments, the induction phase is between or between about 12 and 30 weeks long. In some embodiments, the induction phase is about 12 weeks long. In some embodiments, the induction phase is about 15 weeks long. In some embodiments, the induction phase is about 18 weeks long. In some embodiments, the induction phase is at least 21 weeks long. In some embodiments, the induction phase is about 24 weeks long.

In some embodiments, the induction phase comprises multiple consecutive administrations of gemcitabine without interruption. In some embodiments, the induction phase comprises at least 2, at least 3, at least 4, at least 5, at least 6, at least 7, or 8 consecutive administrations without a rest period. In some embodiments, each administration comprises inserting an intravesical drug delivery system comprising about 150 mg to about 300 mg, about 200 mg to about 250 mg, or about 225 mg of gemcitabine into the bladder and removing the intravesical drug delivery system about three weeks later. In some embodiments, gemcitabine is continuously delivered during the induction phase. In some embodiments, the induction phase precedes the maintenance phase.

In some embodiments, a maintenance phase follows the induction phase. In some embodiments, the maintenance phase comprises administering gemcitabine periodically with rest periods between each administration. In some embodiments, there is a rest phase between the induction phase and the maintenance phase. In some embodiments, the rest phase before the maintenance phase is about 1 month, about 2 months, about 3 months, about 4 months, or about 5 months. In some embodiments, the rest phase between administrations in the maintenance phase is about 1 month, about 2 months, about 3 months, about 4 months, about 5 months, or about 6 months. In some embodiments, the maintenance phase comprises administering gemcitabine quarterly, such as about every three months.

In some embodiments, the maintenance phase continues for at least 6 months, at least 12 months, at least 18 months, or at least 2 years. In some embodiments, the maintenance phase continues for about 12 months or about 18 months. In some embodiments, the total treatment with gemcitabine including the induction phase and the maintenance phase is about two years.

In some aspects, provided herein is a method of treating a bladder cancer in an individual comprising i) administering gemcitabine locally to the bladder of the individual about every three weeks for about at least 9 weeks, at least 15 weeks, at least 18 weeks, at least 21 weeks, or about 24 weeks during an induction phase; ii) administering an effective amount of gemcitabine locally to the bladder of the individual about every three months during a maintenance phase following the induction phase; wherein each administration of gemcitabine comprises continuously delivering gemcitabine to the bladder for at least 7 days during a gemcitabine delivery period. In some embodiments, about 225 mg of gemcitabine is administered. In some embodiments, administering gemcitabine comprises inserting an intravesical drug delivery system comprising about 150 mg to about 300 mg, about 200 mg to about 250 mg, or about 225 mg of gemcitabine into the bladder and removing the intravesical drug delivery system about three weeks later.

In some aspects, provided herein is a method of treating Bacillus Calmette-Guerin (BCG) unresponsive high-risk non-muscle invasive bladder cancer (HR-NMIBC) with carcinoma in situ (CIS) with or without papillary tumors in an individual comprising administering to the bladder of the individual about 225 mg of gemcitabine about every three weeks (Q3W) for at least about 24 weeks, wherein such treatment results in a complete response rate in a population of patients who have received such treatment of at least 50%.

In some aspects, provided herein is a method of treating Bacillus Calmette-Guerin (BCG) unresponsive high-risk non-muscle invasive bladder cancer (HR-NMIBC) with carcinoma in situ (CIS) with or without papillary tumors in an individual comprising administering to the bladder of the individual about 225 mg of gemcitabine about every three weeks (Q3W) for at least about 24 weeks, wherein the individual has a complete response.

In some aspects, provided herein is a method of treating high-risk non-muscle invasive bladder cancer (HR-NMIBC) that is unresponsive to intravesical Bacillus Calmette-Guerin (BCG) therapy in an individual comprising i) a 24 week induction phase comprising administering about 225 mg gemcitabine to the bladder of the individual about every 3 weeks (Q3W) during an administration period, and ii) an 18 month maintenance phase comprising administering about 225 mg gemcitabine to the bladder of the individual about every twelve weeks during an administration period, wherein each administration period comprises a delivery period of at least seven days when gemcitabine is released from an intravesical drug delivery system, and wherein the intravesical drug delivery system is removed about 3 weeks after each administration. In some embodiments, after each administration period in i) the intravesical drug delivery system is removed and replaced with a new intravesical drug delivery system for a total of about 24 weeks.

In some embodiments, the method comprises (i) placing an intravesical drug delivery system into the bladder of the individual, wherein the intravesical drug delivery system remains in the bladder for about three weeks (e.g., 19, 20, 21, 22, or 23 days) and wherein gemcitabine is continuously delivered to the bladder for at least seven days. In some embodiments, provided herein is a method of treating bladder cancer in an individual comprising (i) placing an intravesical drug delivery system into the bladder of the individual, wherein the intravesical drug delivery system remains in the bladder for about three weeks (e.g., 19, 20, 21, 22, or 23 days) and wherein the intravesical drug delivery system delivers the gemcitabine passively. In some embodiments, provided herein is a method of treating bladder cancer in an individual comprising (i) placing a first intravesical drug delivery system into the bladder of the individual on day 0, (ii) removing the first intravesical drug delivery system about 3 weeks (e.g., 19, 20, 21, 22, or 23 days) later, (iii) placing a second intravesical drug delivery system in the bladder of the individual on the same day as the removing of the first intravesical drug delivery system, and (iv) removing the second intravesical drug delivery system three weeks or about three weeks later. In some embodiments, the intravesical drug delivery system comprises about 225 mg of gemcitabine. In some embodiments, the subsequent intravesical drug delivery system is inserted on the same day that the previous intravesical drug delivery system is removed. In some embodiments, the subsequent intravesical drug delivery system is inserted within 1, within 2, within 3, within 4 or within 5 hours of removal of the previous intravesical drug delivery system. In some embodiments, the subsequent intravesical drug delivery system is inserted on a different day than the day that the previous intravesical drug delivery system is removed. In some embodiments, the subsequent intravesical drug delivery system is inserted within 1 day, within 2 days, within 3 days, within 4 days or within 5 days of removal of the previous intravesical drug delivery system.

In some embodiments, the method comprises (i) placing an intravesical drug delivery system into the bladder of the individual, wherein the intravesical drug delivery system remains in the bladder for about three weeks (e.g., 19, 20, 21, 22, or 23 days) and wherein the gemcitabine is continuously delivered to the bladder for at least 7 days. In some embodiments, provided herein is a method of treating bladder cancer in an individual comprising (i) using a urinary placement catheter, transurethrally deploying an intravesical drug delivery system into the bladder of the individual, wherein the intravesical drug delivery system remains in the bladder for about three weeks (e.g., 19, 20, 21, 22, or 23 days) and wherein the intravesical drug delivery system delivers the gemcitabine passively. In some embodiments, provided herein is a method of treating bladder cancer in an individual comprising (i) using a urinary placement catheter, transurethrally deploying a first intravesical drug delivery system into the bladder of the individual on day 0, (ii) using a cystoscope, removing the first intravesical drug delivery system at about three weeks (e.g., 19, 20, 21, 22, or 23 days) later, (iii) using a urinary placement catheter, transurethrally deploying a second intravesical drug delivery system in the bladder of the individual on the same day as the removing of the first intravesical drug delivery system, and (iv) using a cystoscope, removing the second intravesical drug delivery system three weeks or about three weeks later.

In some embodiments, gemcitabine is administered to the bladder of the individual via an intravesical drug delivery system comprising an intravesical device comprising about 225 mg of gemcitabine. In some embodiments, the intravesical delivery device comprises a housing defining a reservoir; a first unit (minitablet) contained within the reservoir, the first unit (minitablet) comprising an antimetabolite; and a second unit (minitablet) contained within the reservoir in a position distinct from the first unit (minitablet), wherein the second unit (minitablet) comprises a functional agent that facilitates in vivo release of the antimetabolite from the housing. In some embodiments, the intravesical device comprises a housing which contains and controllably releases the gemcitabine and is elastically deformable between a retention shape configured to retain the intravesical device in the individual's bladder and a deployment shape for passage of the intravesical device through the individual's urethra.

In some embodiments the intravesical device comprises a housing configured for intravesical insertion and a dosage form comprising gemcitabine, wherein the housing holds the dosage form and is configured to release gemcitabine in an amount effective for the treatment of high-risk non-muscle invasive bladder cancer. In some embodiments, the intravesical device comprises a housing defining a reservoir, a first unit contained within the reservoir comprising gemcitabine, and a second unit (minitablet) comprising a functional agent that facilitates in vivo release of gemcitabine from the housing. In some embodiments, the agent that facilitates release is urea. In some embodiments, the intravesical device comprises about 150 mg to about 300 mg, about 200 mg to about 250 mg, or about 225 mg gemcitabine.

In some embodiments, the intravesical drug delivery system releases gemcitabine at a rate of about 0.5 mg/day to about 50 mg/day, about 1 mg/day to about 40 mg/day, about 3 mg/day to about 30 mg/day, about 5 mg/day to about 20 mg/day, about 10 mg/day to about 40 mg/day, or about 1 mg/day to about 15 mg/day.

In some embodiments, the intravesical device is elastically deformable between a retention shape configured to retain the intravesical device in the individual's bladder and a deployment shape for passage of the intravesical device through the individual's urea. In some embodiments, the retention shape is a pretzel or bi-oval shape. In some embodiments, the deployment shape is suitable for deployment through a catheter. In some embodiments, the intravesical device can be retrieved using a cytoscope and forceps. In some embodiments, gemcitabine is administered to the bladder about every three weeks during the induction phase and then every three months thereafter during the maintenance phase.

In some embodiments, provided herein are methods of treating individuals with high-risk non-muscle invasive bladder cancer comprising administering gemcitabine locally to the bladder of the individual, wherein each gemcitabine administration comprises delivering gemcitabine continuously to the bladder for at least seven days during a gemcitabine delivery period. In some embodiments, gemcitabine is administered to the bladder about every three weeks for up to the first six months and every three months thereafter. In some embodiments, gemcitabine is administered to the bladder about every three weeks for the first 21 weeks and then every three months thereafter for a total of about 2 years. In some embodiments, gemcitabine is administered to the bladder about every three weeks for the first 24 weeks and then every three months thereafter for a total of about 2 years. In some embodiments, about 225 mg of gemcitabine is administered during each administration. In some embodiments, the individual has BCG unresponsive non-muscle invasive bladder cancer. In some embodiments, the individual has refused or is ineligible for radical cystectomy.

In some embodiments, the method of treating individuals with high-risk non-muscle invasive bladder cancer comprises an induction phase and a maintenance phase. In some embodiments, the induction phase comprises administering gemcitabine locally to the bladder multiple times, without interruption. In some embodiments, each gemcitabine administration comprises a gemcitabine delivery period of at least seven days, during which a therapeutically effective amount of gemcitabine is released into the bladder. In some embodiments, administering gemcitabine comprises inserting an intravesical drug delivery system comprising gemcitabine and removing the intravesical drug delivery system about three weeks later. In some embodiments, the gemcitabine delivery period is less than three weeks. For example, in some embodiments, the induction phase comprises administering gemcitabine to the bladder every three weeks without interruption, wherein gemcitabine is delivered to the bladder for two to three weeks. In some embodiments, the induction phase comprises administering gemcitabine to the bladder every three weeks without interruption, wherein gemcitabine is delivered to the bladder for one to three weeks. In some embodiments, gemcitabine is administered to the bladder eight consecutive times without interruption during the induction phase. In some embodiments, gemcitabine is delivered to the bladder by release from an intravesical drug delivery system. In some embodiments, the induction phase is about six months, such as about 24 weeks. In some embodiments, gemcitabine is administered about every three weeks for about 24 weeks during the induction phase. In some embodiments, about 225 mg gemcitabine is administered during each administration. In some embodiments, the individual has BCG unresponsive non-muscle invasive bladder cancer. In some embodiments, the individual has refused or is ineligible for radical cystectomy.

In some embodiments, the induction phase comprises administering gemcitabine on weeks 0, 3, 6, 9, 12, 15, 18, and 21. In some embodiments, the induction phase comprises inserting an intravesical drug delivery system comprising gemcitabine on weeks 0, 3, 6, 9, 12, 15, 18, and 21 and removing each intravesical drug delivery system about three weeks later on weeks 3, 6, 9, 12, 15, 18, 21, and 24. In some embodiments, the induction phase comprises inserting a first intravesical drug delivery system comprising gemcitabine into the bladder in week 0 and removing the first intravesical drug delivery system in week 3, inserting a second intravesical drug delivery system comprising gemcitabine into the bladder in week 3 and removing the second intravesical drug delivery system in week 6, inserting a third intravesical drug delivery system comprising gemcitabine into the bladder in week 6 and removing the third intravesical drug delivery system in week 9, inserting a fourth intravesical drug delivery system comprising gemcitabine into the bladder in week 9 and removing the intravesical drug delivery system device in week 12, inserting a fifth intravesical drug delivery system comprising gemcitabine into the bladder in week 12 and removing the fifth intravesical drug delivery system in week 15, inserting a sixth intravesical drug delivery system comprising gemcitabine into the bladder in week 15 and removing the sixth intravesical drug delivery system in week 18, inserting a seventh intravesical drug delivery system comprising gemcitabine into the bladder in week 18 and removing the seventh intravesical drug delivery system in week 21, inserting an eighth intravesical drug delivery system in week 21 and removing the eighth intravesical drug delivery system in week 24. In some embodiments, the intravesical drug delivery system comprises about 150 mg to about 300 mg, about 200 mg to about 250 mg, or about 225 mg gemcitabine. In some embodiments, the intravesical drug delivery system remains in the bladder for about three weeks. In some embodiments, the individual has BCG unresponsive non-muscle invasive bladder cancer. In some embodiments, the individual has refused or is ineligible for radical cystectomy.

In some embodiments, a maintenance phase follows the induction phase. In some embodiments, there is a rest period between the induction phase and the maintenance phase, during which gemcitabine is not administered. In some embodiments, the rest period is about three to about five months. In some embodiments, the rest period is about 12 weeks. In some embodiments, gemcitabine is administered less frequently in the maintenance phase than in the induction phase. In some embodiments, the maintenance phase comprises rest periods during which no gemcitabine is delivered to the bladder of the individual. In some embodiments, the maintenance phase comprises delivering gemcitabine to the bladder of the individual quarterly or about every three months. In some embodiments, the intravesical drug delivery system comprises about 150 mg to about 300 mg, about 200 mg to about 250 mg, or about 225 mg gemcitabine. In some embodiments, the individual has BCG unresponsive non-muscle invasive bladder cancer. In some embodiments, the individual has refused or is ineligible for radical cystectomy.

In some embodiments, the maintenance phase comprises inserting an intravesical drug delivery system comprising gemcitabine into the bladder of the individual about every three months. In some embodiments, the intravesical drug delivery system remains in the bladder for about three weeks. In some embodiments, the intravesical drug delivery system comprises about 150 mg to about 300 mg, about 200 mg to about 250 mg, or about 225 mg gemcitabine. In some embodiments, the maintenance phase comprises administering gemcitabine to the bladder of the individual in weeks 36, 48, 60, 72, 84, and 96 of a dosing schedule. In some embodiments, the maintenance phase comprises inserting an intravesical drug delivery system comprising gemcitabine to the bladder of the individual in weeks 36, 48, 60, 72, 84, and 96 of a dosing schedule, and removing the intravesical drug delivery system in weeks 39, 51, 63, 75, 87, and 99 after an about three week indwelling period. In some embodiments, the individual has BCG unresponsive non-muscle invasive bladder cancer. In some embodiments, the individual has refused or is ineligible for radical cystectomy.

Also provided herein is a method of treating high-risk non-muscle invasive bladder cancer in an individual comprising administering gemcitabine locally to the bladder of the individual about every 12 weeks, such as quarterly. In some embodiments, each gemcitabine administration is about three weeks. In some embodiments, each gemcitabine administration comprises a gemcitabine delivery period of at least seven days, during which a therapeutically effective amount of gemcitabine is released into the bladder. In some embodiments, administering gemcitabine comprises inserting an intravesical drug delivery system comprising gemcitabine and removing the intravesical drug delivery system about three weeks later. In some embodiments, gemcitabine is administered about every 12 weeks for at least six months. In some embodiments, gemcitabine is administered every about 12 weeks for at least one year. In some embodiments, gemcitabine is administered every about 12 weeks for about 18 months. In some embodiments, about 225 mg of gemcitabine is administered to the individual during each administration. In some embodiments, the individual has BCG unresponsive non-muscle invasive bladder cancer. In some embodiments, the individual has refused or is ineligible for radical cystectomy.

Also provided herein is a method of treating high-risk non-muscle invasive bladder cancer in an individual comprising a dosing schedule of at least 21 weeks comprising administering about 225 mg gemcitabine locally to the bladder about every three weeks. In some embodiments, administering about 225 mg gemcitabine about every three weeks comprises inserting an intravesical drug delivery system into the bladder and removing the intravesical drug delivery system about three weeks later, wherein the intravesical drug delivery system comprises about 150 mg to about 300 mg, about 200 mg to about 250 mg, or about 225 mg gemcitabine. In some embodiments, the intravesical drug delivery system continuously delivers gemcitabine to the bladder during a gemcitabine delivery period. In some embodiments, the gemcitabine delivery period is at least 7 days. In some embodiments, the gemcitabine delivery period is about three weeks or less. In some embodiments, the gemcitabine delivery period is at least one week or at least two weeks. In some embodiments, the gemcitabine delivery period is about one to about two weeks. In some embodiments, less than 225 mg of gemcitabine is delivered to the individual. In some embodiments, not all of the gemcitabine contained in the intravesical drug delivery system is released into the bladder. In some embodiments, the individual has BCG unresponsive non-muscle invasive bladder cancer. In some embodiments, the individual has refused or is ineligible for radical cystectomy.

In some embodiments, provided herein is a method of treating high-risk non-muscle invasive bladder cancer in an individual comprising inserting an intravesical drug delivery system comprising about 150 mg to about 300 mg, about 200 mg to about 250 mg, or about 225 mg gemcitabine into the bladder and removing the intravesical drug delivery system about three weeks later, wherein on the day that the intravesical drug delivery system is removed, a new intravesical drug delivery system comprising 225 mg gemcitabine is inserted into the bladder. In some embodiments, an intravesical drug delivery system is inserted and removed eight consecutive times, without interruption. For example, in some embodiments, an intravesical drug delivery system is inserted in week 0, 3, 6, 9, 12, 15, 18, and 21 and the intravesical drug delivery system is removed in weeks 3, 6, 9, 12, 15, 18, 21, and 24. In some embodiments, the individual has BCG unresponsive non-muscle invasive bladder cancer. In some embodiments, the individual has refused or is ineligible for radical cystectomy.

In some embodiments, the method comprises administering gemcitabine on weeks 0, 3, 6, 9, 12, 15, 18, 21, 36, 48, 60, 72, 84, and 96. In some embodiments, administering gemcitabine comprises inserting an intravesical drug delivery system comprising gemcitabine into the bladder and removing the intravesical drug delivery system about three weeks later. In some embodiments, the intravesical drug delivery system comprises about 150 mg to about 300 mg, about 200 mg to about 250 mg, or about 225 mg gemcitabine. In some embodiments, the intravesical drug delivery system releases gemcitabine during a gemcitabine delivery period of at least one week. In some embodiments, the gemcitabine delivery period is at least two weeks. In some embodiments, the gemcitabine delivery period is less than three weeks. In some embodiments, the individual has BCG unresponsive non-muscle invasive bladder cancer. In some embodiments, the individual has refused or is ineligible for radical cystectomy.

In some embodiments, the method comprises administering gemcitabine by inserting an intravesical drug delivery system comprising gemcitabine into the bladder and removing the intravesical drug delivery system about three weeks later. In some embodiments, the intravesical drug delivery system is administered every 3 weeks through Week 24, and then every 12 weeks until Week 96.

In some embodiments, the method comprises a dosing schedule of at least 99 weeks comprising i) inserting a first intravesical drug delivery system into the bladder in week 0, and removing the first intravesical drug delivery system in week 3; ii) inserting a second intravesical drug delivery system into the bladder in week 3 after removing the first intravesical drug delivery system, and removing the second intravesical drug delivery system in week 6; iii) inserting a third intravesical drug delivery system into the bladder in week 6 after removing the second intravesical drug delivery system, and removing the third intravesical drug delivery system in week 9; iv) inserting a fourth intravesical drug delivery system into the bladder in week 9 after removing the third intravesical drug delivery system, and removing the fourth intravesical drug delivery system in week 12; v) inserting a fifth intravesical drug delivery system into the bladder in week 12 after removing the fourth intravesical drug delivery system, and removing the fifth intravesical drug delivery system in week 15; vi) inserting a sixth intravesical drug delivery system into the bladder in week 15 after removing the fifth intravesical drug delivery system, and removing the sixth intravesical drug delivery system in week 18; vii) inserting a seventh intravesical drug delivery system into the bladder in week 18 after removing the sixth intravesical drug delivery system, and removing the seventh intravesical drug delivery system in week 21; viii) inserting an eighth intravesical drug delivery system into the bladder in week 21 after removing the seventh intravesical drug delivery system, and removing the eighth intravesical drug delivery system in week 24; ix) inserting a ninth intravesical drug delivery system into the bladder in week 36, and removing the ninth intravesical drug delivery system in week 39; x) inserting a tenth intravesical drug delivery system into the bladder in week 48, and removing the tenth intravesical drug delivery system in week 51; xi) inserting an eleventh intravesical drug delivery system into the bladder in week 60, and removing the eleventh intravesical drug delivery system in week 63; xi) inserting a twelfth intravesical drug delivery system into the bladder in week 72, and removing the twelfth intravesical drug delivery system in week 75; xi) inserting a thirteenth intravesical drug delivery system into the bladder in week 84, and removing the thirteenth intravesical drug delivery system in week 87; xi) inserting a fourteenth intravesical drug delivery system into the bladder in week 96, and removing the fourteenth intravesical drug delivery system in week 99; wherein each intravesical drug delivery system comprises an intravesical device comprising about 225 mg of gemcitabine.

In some embodiments, the method comprises i) deploying an intravesical drug delivery system into the bladder once every 3 weeks over approximately 6 months; followed by ii) deploying an intravesical drug delivery system into the bladder once every 12 weeks over approximately 18 months; wherein each intravesical drug delivery system is removed 3 weeks after each administration. In some embodiments, the method comprises deploying a total of about 14 intravesical drug delivery systems over about 2 years.

In some embodiments, provided herein is a method of treating high-risk non-muscle invasive bladder cancer in an individual comprising administering gemcitabine locally to the bladder of the individual, wherein gemcitabine administration comprises delivering gemcitabine continuously to the bladder for at least 7 days during a gemcitabine delivery period, wherein during the gemcitabine delivery period, there is an effective amount of gemcitabine in the urine of the individual for treating high-risk non-muscle invasive bladder cancer. In some embodiments, the urine concentration of gemcitabine during the delivery period is about 1 μg/mL to about 25 μg/mL, about 1 μg/mL to about 20 μg/mL, about 1 μg/mL to about 15 μg/mL, about 1 μg/mL to about 10 μg/mL. In some embodiments, the urine concentration of gemcitabine during the delivery period is about 18.4 μg/mL. In some embodiments, the maximum urine concentration of gemcitabine during the delivery period is about 18.4 μg/mL. In some embodiments, the urine concentration of gemcitabine during the delivery period is about 16.1 μg/mL. In some embodiments, the urine concentration of gemcitabine during the delivery period is about 8.9 μg/mL. In some embodiments, the urine concentration of gemcitabine during the delivery period is about 6.9 μg/mL. In some embodiments, the concentration of gemcitabine in the urine reaches a maximum concentration between about 2 to about 4 days following administration of the gemcitabine. In some embodiments, the concentration of gemcitabine in the urine reaches a maximum concentration about 2 days, about 3 days, or about 4 days following administration of the gemcitabine. In some embodiments, the mean cumulative excreted gemcitabine in urine expressed as percentage of the administered dose from 0-168 hours is between about 36% to about 54%. In some embodiments, the mean cumulative excreted gemcitabine in urine expressed as percentage of the administered dose from 0-168 hours is between about 35% to about 40%, about 40% to about 45%, about 45% to about 50%, or about 50% to about 55%. In some embodiments, the mean cumulative excreted gemcitabine in urine expressed as percentage of the administered dose from 0-168 hours is between about 36.2% to about 53.8%. In some embodiments, the deaminated metabolite of gemcitabine, dFdU, is detected in urine following administration of gemcitabine to the bladder of an individual. In some embodiments, the mean cumulative excreted dFdU amount in urine expressed as percentage of the administered dose from 0-168 hours is between about 11% to about 23%. In some embodiments, the mean cumulative excreted dFdU amount in urine expressed as percentage of the administered dose from 0-168 hours is between about 10% to about 15%, about 15% to about 20%, or about 20% to about 25%. In some embodiments, the mean cumulative excreted dFdU amount in urine expressed as percentage of the administered dose from 0-168 hours is between about 11.1% to about 23.3%. In some embodiments, the delivery period is less than three weeks. In some embodiments, the delivery period is about one to about three weeks. In some embodiments, the delivery period is about one to about two weeks. In some embodiments, administering gemcitabine comprises inserting an intravesical drug delivery system comprising gemcitabine into the bladder of the individual and removing the intravesical drug delivery period about three weeks later. In some embodiments, the intravesical drug delivery system releases about 70% of the total gemcitabine in the first seven days of administration. In some embodiments, the intravesical drug delivery system releases about 90% of the total gemcitabine by day 21 of administration. In some embodiments, the intravesical drug delivery system releases gemcitabine during a first phase of the delivery at a first release rate followed by a second phase of the delivery having a second release rate. In some embodiments, gemcitabine is administered to the bladder about every three weeks for the first 24 weeks and then every three months thereafter for a total of about two years. In some embodiments, 225 mg of gemcitabine is administered during each administration. In some embodiments, the individual has BCG unresponsive non-muscle invasive bladder cancer. In some embodiments, the individual has refused or is ineligible for radical cystectomy.

In some embodiments, provided herein is a method of treating high-risk non-muscle invasive bladder cancer in an individual comprising administering gemcitabine locally to the bladder of the individual, wherein gemcitabine administration comprises delivering gemcitabine continuously to the bladder for at least 7 days during a gemcitabine delivery period, wherein the gemcitabine is delivered via an intravesical drug delivery system comprising an intravesical device and a drug, such as gemcitabine. In some embodiments the intravesical device comprises a housing configured for intravesical insertion and a dosage form comprising gemcitabine, wherein the housing holds the dosage form and is configured to release gemcitabine in an amount effective for the treatment of high-risk non-muscle invasive bladder cancer. In some embodiments, the intravesical device comprises a housing defining a reservoir, a first unit contained within the reservoir comprising gemcitabine, and a second unit comprises a functional agent that facilitates in vivo release of gemcitabine from the housing. In some embodiments, the agent that facilitates release is urea. In some embodiments, the intravesical device comprises about 150 mg to about 300 mg, about 200 mg to about 250 mg, or about 225 mg gemcitabine. In some embodiments, the intravesical drug delivery system releases about 0.5 mg/day to about 50 mg/day, about 1 mg/day to about 40 mg/day, about 3 mg/day to about 30 mg/day, about 5 mg/day to about 20 mg/day or about 1 mg/day to about 15 mg/day. In some embodiments, the intravesical device is elastically deformable between a retention shape configured to retain the intravesical device in the individual's bladder and a deployment shape for passage of the intravesical device through the individual's urea. In some embodiments, the retention shape is a pretzel or bi-oval shape. In some embodiments, the deployment shape is suitable for deployment through a catheter. In some embodiments, the intravesical device can be retrieved using a cytoscope and forceps. In some embodiments, gemcitabine is administered to the bladder about every three weeks for the first 24 weeks and then every three months thereafter for a total of about 2 years. In some embodiments, 225 mg of gemcitabine is administered during each administration. In some embodiments, the individual has BCG unresponsive non-muscle invasive bladder cancer. In some embodiments, the individual has refused or is ineligible for radical cystectomy.

In some embodiments, provided herein is a method of treating high-risk non-muscle invasive bladder cancer in an individual comprising administering gemcitabine locally to the bladder of the individual, wherein gemcitabine administration comprises delivering gemcitabine continuously to the bladder for at least 7 days during a gemcitabine delivery period, wherein the individual has carcinoma in situ. In some embodiments, the individual has high grade disease. In some embodiments, the individual has high grade Ta or T1 disease. In some embodiments, the individual has papillary disease. In some embodiments, gemcitabine is administered to the bladder about every three weeks for the first 24 weeks and then about every three months thereafter for a total of about two years. In some embodiments, about 225 mg of gemcitabine is administered during each administration. In some embodiments, the individual has BCG unresponsive non-muscle invasive bladder cancer. In some embodiments, the individual has received previous intravesical BCG therapy. In some embodiments, BCG therapy comprises repeated instillations of BCG over the course of three or six weeks.

In some embodiments, the individual with high-risk non muscle invasive bladder cancer is ineligible for or has elected not to undergo a radical cystectomy. In some embodiments, gemcitabine is administered to the bladder of the individual every about three weeks for the first 24 weeks and then about every three months thereafter for a total of about two years. In some embodiments, about 225 mg of gemcitabine is administered during each administration. In some embodiments, the individual has BCG unresponsive non-muscle invasive bladder cancer. In some embodiments, the individual has refused or is ineligible for radical cystectomy.

In some embodiments, the present methods comprising locally administering gemcitabine to the bladder result in reduced side effects compared to alternative treatments for high-risk non muscle invasive bladder cancer. In some embodiments, locally administering gemcitabine to the bladder comprises administering gemcitabine directly within the bladder. In some embodiments, the methods reduce the incidence of adverse effects, micturition urgency, pollakiuria, dysuria, noninfective cystitis, urinary tract infection, pruritus, and/or diarrhea compared to alternative therapies. In some embodiments, about 91% of individuals within the population of patients experience an adverse effect. In some embodiments, less than about 91% of individuals within the population of patients experience an adverse effect. In some embodiments, between about 75% to about 91% of individuals within the population of patients experience an adverse effect. In some embodiments, about 30% of individuals within the population of patients experience a greater than grade 3 adverse effect. In some embodiments, less than about 35% of individuals within the population of patients experience a greater than grade 3 adverse effect. In some embodiments, between about 20% to about 35% of individuals within the population of patients experience a greater than grade 3 adverse effect. In some embodiments about 35% of individuals within the population of patients experience micturition urgency. In some embodiments, less than about 40% of individuals within the population of patients experience micturition urgency. In some embodiments, between about 25% to about 40% of individuals within the population of patients experience micturition urgency. In some embodiments about 35% of individuals within the population of patients experience pollakiuria. In some embodiments, less than about 40% of individuals within the population of patients experience pollakiuria. In some embodiments, between about 25% to about 40% of individuals within the population of patients experience pollakiuria. In some embodiments about 26% of individuals within the population of patients experience dysuria. In some embodiments, less than about 30% of individuals within the population of patients experience dysuria. In some embodiments, between about 15% to about 30% of individuals within the population of patients experience dysuria. In some embodiments about 22% of individuals within the population of patients experience noninfective cystitis. In some embodiments, less than about 30% of individuals within the population of patients experience noninfective cystitis. In some embodiments, between about 15% to about 30% of individuals within the population of patients experience noninfective cystitis. In some embodiments about 22% of individuals within the population of patients experience a urinary tract infection. In some embodiments, less than about 30% of individuals within the population of patients experience a urinary tract infection. In some embodiments, between about 15% to about 30% of individuals within the population of patients experience a urinary tract infection. In some embodiments, less than 5% of individuals within the population of patients experience pruritus. In some embodiments, between about 0% to about 5% of individuals within the population of patients experience pruritus. In some embodiments, less than 5% of individuals within the population of patients experience diarrhea. In some embodiments, between about 0% to about 5% of individuals within the population of patients experience diarrhea. In some embodiments about 11% of individuals within the population of patients experience hematuria. In some embodiments about 9% of individuals within the population of patients experience hematuria. In some embodiments, less than about 15% of individuals within the population of patients experience hematuria. In some embodiments, between about 0% to about 15% of individuals within the population of patients experience hematuria. In some embodiments about 6% of individuals within the population of patients experience urinary tract pain. In some embodiments, less than about 10% of individuals within the population of patients experience urinary tract pain. In some embodiments, between about 0% to about 10% of individuals within the population of patients experience urinary tract pain. In some embodiments about 4% of individuals within the population of patients experience urinary retention. In some embodiments, less than about 10% of individuals within the population of patients experience urinary retention. In some embodiments, between about 0% to about 10% of individuals within the population of patients experience urinary retention. In some embodiments about 2% of individuals within the population of patients experience renal impairment. In some embodiments, less than about 5% of individuals within the population of patients experience renal impairment. In some embodiments, between about 0% to about 5% of individuals within the population of patients experience renal impairment. In some embodiments about 2% of individuals within the population of patients experience urosepsis. In some embodiments, less than about 5% of individuals within the population of patients experience urosepsis. In some embodiments, between about 0% to about 5% of individuals within the population of patients experience urosepsis.

The present application in one aspect provides a method of treating bladder cancer in an individual comprising administering to the individual an effective amount of an antibody that binds to PD-1. In some embodiments, the method comprises administering (e.g., via intravenous infusion) to the individual about 360 mg of an antibody that binds to PD-1 about every three weeks, and the antibody that binds to PD-1 is administered at least twice. In some embodiments, the antibody that binds to PD-1 comprises a variable heavy chain (VH) and a variable light chain (VL), wherein the VH comprises a CDRH1 comprising the amino acid sequence set forth in SEQ ID NO:1, a CDRH2 comprising the amino acid sequence set forth in SEQ ID NO: 2, a CDRH3 comprising the amino acid sequence set forth in SEQ ID NO:3, and the VL comprises a CDRL1 comprising the amino acid sequence set forth in SEQ ID NO: 4, a CDRL2 comprising the amino acid sequence set forth in SEQ ID NO: 5, and a CDRL3 comprising the amino acid sequence set forth in SEQ ID NO: 6. In some embodiments, the antibody that binds to PD-1 is Cetrelimab.

In some aspects, provided herein is a method of treating a bladder cancer in an individual comprising administering an antibody that binds to PD-1 comprising a variable heavy chain (VH) and a variable light chain (VL), wherein the VH comprises a CDRH1 comprising the amino acid sequence set forth in SEQ ID NO:1, a CDRH2 comprising the amino acid sequence set forth in SEQ ID NO:2, a CDRH3 comprising the amino acid sequence set forth in SEQ ID NO:3, and the VL comprises a CDRL1 comprising the amino acid sequence set forth in SEQ ID NO: 4, a CDRL2 comprising the amino acid sequence set forth in SEQ ID NO: 5, and a CDRL3 comprising the amino acid sequence set forth in SEQ ID NO: 6 to the individual.

In some embodiments, there is provided a method of treating a bladder cancer in an individual comprising (i) administering to the individual an effective amount of an antibody that binds to PD-1. In some embodiments, the antibody that binds to PD-1 is administered multiple times. In some embodiments, the antibody that binds to PD-1 is administered twice, 3 times, 4 times, 5 times, 6 times, 7, times, 8 times, 9 times, 10 times, 11 times, 12 times, 13 times, 14 times, 15 times, 16 times, 17 times, 18 times, 19 times, or 20 times. In some embodiments, the antibody that binds to PD-1 is administered weekly, every 2 weeks, every 3 weeks, every 4 weeks, monthly or every 2 months. In some embodiments, about 120 mg, about 240 mg, about 360 mg, or about 480 mg of the antibody that binds to PD-1 is administered.

In some aspects, provided herein is a method of treating a bladder cancer in an individual comprising administering to the individual an antibody that binds to PD-1 about every three weeks for about 78 weeks. In some embodiments, administration of the antibody that binds to PD-1 occurs every 3 weeks for at least 24 weeks. In some embodiments, the method comprises administering the antibody that binds to PD-1 on weeks 0, 3, 6, 9, 12, 15, 18, 21, 24, 27, 30, 33, 36, 39, 42, 45, 48, 51, 54, 57, 60, 63, 66, 69, 72, 75, and 78 of a dosing schedule. In some embodiments, administration of the antibody that binds to PD-1 occurs every 3 weeks for between about 3 and 24, about 24 and 48, about 48 and 72, or about 72 and 96 weeks. In some embodiments, administration of the antibody that binds to PD-1 occurs every 3 weeks for about 78 weeks. In some embodiments, about 360 mg of the antibody that binds to PD-1 is administered (e.g., via intravenous infusion). In some embodiments, the individual has BCG unresponsive non-muscle invasive bladder cancer. In some embodiments, the individual has refused or is ineligible for radical cystectomy.

In some embodiments, provided herein are methods of treating individuals with high-risk non-muscle invasive bladder cancer comprising administering to the individual an antibody that binds to PD-1. In some embodiments, the antibody that binds to PD-1 is administered about every three weeks for the first 18 months. In some embodiments, about 360 mg of the PD-1 antibody is administered (e.g., via intravenous infusion) during each administration. In some embodiments, the individual has BCG unresponsive non-muscle invasive bladder cancer. In some embodiments, the individual has refused or is ineligible for radical cystectomy.

In some embodiments, provided herein are methods of treating individuals with high-risk non-muscle invasive bladder cancer comprising administering an antibody that binds to PD-1 comprising a variable heavy chain (VH) and a variable light chain (VL), wherein the VH comprises a CDRH1 comprising the amino acid sequence set forth in SEQ ID NO:1, a CDRH2 comprising the amino acid sequence set forth in SEQ ID NO:2, a CDRH3 comprising the amino acid sequence set forth in SEQ ID NO:3; and the VL comprises a CDRL1 comprising the amino acid sequence set forth in SEQ ID NO: 4, a CDRL2 comprising the amino acid sequence set forth in SEQ ID NO: 5, and a CDRL3 comprising the amino acid sequence set forth in SEQ ID NO: 6 to the individual. In some embodiments, the PD-1 antibody has a VH comprising the amino acid sequence set forth in SEQ ID NO: 7 and a VL comprising the amino acid sequence set forth in SEQ ID NO:8. In some embodiments, the antibody comprises a heavy chain comprising the amino acid sequence set forth in SEQ ID NO:9 and a light chain comprising the amino acid sequence set forth in SEQ ID NO: 10. In some embodiments, the antibody is cetrelimab. In some embodiments, the antibody has an IgG4 isotype. In some embodiments, the antibody is a full length antibody. In some embodiments, the PD-1 antibody is a human antibody. In some embodiments, the antibody comprises a S228P mutation. In some embodiments, the antibody that binds to PD-1 inhibits PD-1. In some embodiments, the antibody that binds to PD-1 enhances pro-inflammatory cytokine production from T-cells. In some embodiments, the antibody that binds to PD-1 is administered at a dose of about 360 mg. In some embodiments, the antibody that binds to PD-1 is administered about every three weeks. In some embodiments, the antibody that binds to PD-1 is administered systemically. In some embodiments, the antibody that binds to PD-1 is administered intravenously. In some embodiments, the antibody that binds to PD-1 is administered at least twice, at least three times, at least four times or at least ten times. In some embodiments, the antibody that binds to PD-1 is delivered about every three weeks for about 18 months. In some embodiments, the antibody that binds to PD-1 is delivered about every three weeks for the first about 18 months of a two year dosing schedule. In some embodiments, the individual has BCG unresponsive non-muscle invasive bladder cancer. In some embodiments, the individual has refused or is ineligible for radical cystectomy.

Also provided herein is a method of treating high-risk non-muscle invasive bladder cancer in an individual comprising administering to the individual an antibody that binds to PD-1 about every three weeks. In some embodiments, about 360 mg of the antibody that binds to PD-1 is administered to the individual during each administration. In some embodiments, the individual has BCG unresponsive non-muscle invasive bladder cancer. In some embodiments, the individual has refused or is ineligible for radical cystectomy.

Also provided herein is a method of treating high-risk non-muscle invasive bladder cancer in an individual comprising a dosing schedule of at least 24 weeks comprising administering (e.g., via intravenous infusion) 360 mg of an antibody that binds to PD-1 about every three weeks. In some embodiments, the individual has BCG unresponsive non-muscle invasive bladder cancer. In some embodiments, the individual has refused or is ineligible for radical cystectomy.

In some embodiments, provided herein is a method of treating high-risk non-muscle invasive bladder cancer in an individual comprising administering an antibody that binds to PD-1, wherein the individual has carcinoma in situ. In some embodiments, the individual has high grade disease. In some embodiments, the individual has high grade Ta or T1 disease. In some embodiments, the individual has papillary disease. In some embodiments, the antibody that binds to PD-1 is administered about every three weeks for about the first 18 months. In some embodiments, about 360 mg of the PD-1 antibody is administered (e.g., via intravenous infusion) during each administration. In some embodiments, the individual has BCG unresponsive non-muscle invasive bladder cancer. In some embodiments, the individual has received previous intravesical BCG therapy. In some embodiments, BCG therapy comprises repeated instillations of BCG over the course of three or six weeks.

In some embodiments, the individual with high-risk non muscle invasive bladder cancer is ineligible for or has elected not to undergo a radical cystectomy. In some embodiments, the antibody that binds to PD-1 is administered about every three weeks for the first 18 months. In some embodiments, about 360 mg of the PD-1 antibody is administered (e.g., via intravenous infusion) during each administration. In some embodiments, the individual has BCG unresponsive non-muscle invasive bladder cancer. In some embodiments, the individual has refused or is ineligible for radical cystectomy.

In some embodiments, the individual is ineligible for radical cystectomy under the National Comprehensive Cancer Network (NCCN) guidelines. For example, the individual may be unfit for curative therapy due to frailty. Prior to the present methods, such individuals typically received palliative radiation without chemotherapy (3.5 Gy/fraction—10 treatments; or 7Gy/fraction—7 treatments; TURBT; or no treatment).

In some embodiments, the individual cannot tolerate radical cystectomy based upon the American Society of Anesthesiology (ASA) guidelines. For example, the individual who cannot tolerate radial cystectomy may be deemed medically unfit for surgery requiring general or epidural anesthesia.

In other embodiments, the individual may not be suitable for radical cystectomy due to a lack of post-operative care infrastructure (e.g., as determined by the Comprehensive Geriatric Assessment provided by the American Society of Anesthesiologists). In some embodiments, an individual is not suitable for radical cystectomy due to frailty (e.g., as determined by the Comprehensive Geriatric Assessment provided by the American Society of Anesthesiologists). Under these guidelines, an individual is deemed frail if he or she shows abnormal independent activities of daily living, severe malnutrition, cognitive impairment, or comorbidities cumulative illness rating scale for geriatrics (CISR-G) grades 3-4.

### C. Endpoints

The various aspects and embodiments described in this section in the context of a method of treatment comprising administering gemcitabine also apply to gemcitabine for use according to the methods described herein, and minitablets comprising gemcitabine for use according to the methods described herein. Similarly, the various aspects and embodiments described in this section in the context of a method of treatment comprising administering gemcitabine also apply to gemcitabine for use with an intravesical drug delivery system, according to the methods described herein, and gemcitabine for use according to the methods described herein, in combination with an intravesical drug delivery system, and gemcitabine for use according to the methods described herein, wherein the gemcitabine is administered by an intravesical drug delivery system.

In some embodiments, provided herein is a method of treating high-risk non-muscle invasive bladder cancer in an individual comprising administering gemcitabine locally to the bladder of the individual, wherein gemcitabine administration comprises delivering gemcitabine continuously to the bladder for at least 7 days during a gemcitabine delivery period, wherein the individual experiences a complete response (CR). In some embodiments, the complete response is measured by determining the presence of high-grade disease using results from cystoscopy and urine cytology. In some embodiments, a complete response is defined as a negative cystoscopy and negative urine cytology or positive cystoscopy with benign or low grade non-muscle invasive bladder cancer and negative urine cytology. In some embodiments, the complete response rate is determined from the treated analysis set. In some embodiments, the treated analysis set includes the evaluable analysis set and individuals who discontinued from the study. In some embodiments, the evaluable analysis set is defined as all treated participants who had active disease at baseline and adequate disease assessment post-baseline, or who had progressed, died, or was withdrawn from treatment due to recurrence of high-risk disease or progressive disease. In some embodiments, the complete response rate is determined from the evaluable analysis set. In some embodiments, the individual has a durable complete response. In some embodiments the overall CR rate will be measured by determining the proportion of participants without presence of high-grade disease using results from cystoscopy and centrally read urine cytology at any timepoint. In some embodiments, individuals who remain free of recurrence of high grade disease 12 months after treatment have achieved a durable complete response. In some embodiments, gemcitabine is administered to the bladder of the individual about every three weeks for the first 24 weeks and then about every three months thereafter for a total of about two years. In some embodiments, about 225 mg of gemcitabine is administered during each administration. In some embodiments, the individual has BCG unresponsive non-muscle invasive bladder cancer. In some embodiments, the individual has refused or is ineligible for radical cystectomy.

In some embodiments, provided herein is a method of treating high-risk non-muscle invasive bladder cancer in a population of patients comprising administering gemcitabine locally to the bladder of each patient, wherein gemcitabine administration comprises delivering gemcitabine continuously to the bladder for at least 7 days during a gemcitabine delivery period, wherein such treatment results in a complete response (CR) rate in a population of patients who have received such treatment of at least 50%. In some embodiments the complete response rate in a population of patients who have received such treatment is between about 50% and 60%, about 60% and 70%, about 70% and 80%, about 80% and 90%, about 90% and 100%. In some embodiments, the complete response rate in a population of patients who have received such treatment is about 81%. In some embodiments, the complete response rate in a population of patients who have received such treatment is about 82%. In some embodiments, the complete response rate in a population of patients who have received such treatment is about 82%, wherein the complete response is determined from the evaluable analysis set. In some embodiments, the complete response rate in a population of patients who have received such treatment is about 87%, wherein the complete response is determined from the evaluable analysis set. In some embodiments, the complete response rate in a population of patients who have received such treatment is about 83%. In some embodiments, the complete response rate in a population of patients who have received such treatment is about 83%, wherein the complete response is determined from the full analysis set. In some embodiments, the complete response rate in a population of patients who have received such treatment is about 79%. In some embodiments, the complete response rate in a population of patients who have received such treatment is about 79%, wherein the complete response is determined from the treated analysis set. In some embodiments, the complete response rate in a population of patients who have received such treatment is about 77%. In some embodiments, the complete response rate in a population of patients who have received such treatment is about 77%, wherein the complete response is determined from the treated analysis set. In some embodiments, the complete response rate in a population of patients who have received such treatment is about 73%. In some embodiments, the complete response rate in a population of patients who have received such treatment is about 73%, wherein the complete response is determined from the treated analysis set. In some embodiments, the CR in a population of patients who have received such treatment is about 78%. In some embodiments, the CR in a population of patients who have received such treatment is greater than about 51%. In some embodiments, the CR in a population of patients who have received such therapy is about 51% to about 87%. In some embodiments, the CR in a population of patients who have received such therapy is about 70% to about 90%. In some embodiments, the CR in a population of patients who have received such therapy is about 72%, about 73%, about 74%, about 75%, about 76%, about 77%, or about 78%. In some embodiments, the CR is about 70% to about 80%. In some embodiments, the complete response rate in the population of patients is between about 71% to about 91%. In some embodiments, the 95% confidence interval of a complete response rate in the population of patients is between about 71% to about 91%. In some embodiments, the 95% confidence interval of a complete response rate in the population of patients is between about 70% to about 90%. In some embodiments, the individual has a complete response within 3 months following the start of administration of gemcitabine. In some embodiments, the population of patients comprises individuals with high-risk non-muscle invasive bladder cancer. In some embodiments, the population of patients comprises individuals with CIS. In some embodiments, about 60% to about 80%, such as about 70% of individuals within the population of patients have CIS. In some embodiments, the population of patients comprises individuals who have CIS and Ta disease. In some embodiments, about 20% to about 40%, such as about 30% of individuals within the population of patients have CIS and Ta disease. In some embodiments, the population of patients comprises CIS and T1 disease. In some embodiments, the population of patients comprises individuals who are unresponsive to BCG therapy. In some embodiments, the median total doses of prior BCG in the population of patients is about 12. In some embodiments, the population of patients comprises individuals who have received 7 to 24 prior doses of BCG. In some embodiments, the median time from last BCG to CIS diagnosis in the population of patients is about 3 months. In some embodiments, the population of patients comprises individuals who have received BCG between 1 and 22 months prior to the start of treatment with gemcitabine. In some embodiments, the population of patients comprises individuals who do not receive a radical cystectomy. In some embodiments, the majority of individuals in the population of patients declined radical cystectomy. In some embodiments, about 96% of individuals within the population of patients decline radical cystectomy. In some embodiments, about 4% of individuals within the population of patients are ineligible for a radical cystectomy. In some embodiments, the median age in the population of patients is over 65. In some embodiments, the median age in the population of patients is about 72. In some embodiments, the majority of individuals in the population of patients are male. In some embodiments, about 83% of individuals within the population of patients are male. In some embodiments, about 96% of individuals within the population of patients have an ECOG performance status 0.

In some embodiments, such treatments results in a 12-month CR rate in the population of patients of at least about 40%. In some embodiments, the 12-month CR rate in the population of patients is between about 43% to about 79%. In some embodiments, the 95% confidence interval of a 12-month CR rate in the population of patients is between about 43% to about 79%. In some embodiments, the 12-month CR rate in the population of patients is between about 40% to about 50%, about 50% to about 60%, about 60% to about 70%, about 70% to about 80%. In some embodiments, the 12-month CR rate in the population of patients is about 64%. In some embodiments, the 12-month CR rate is calculated from all treated patients in the population of patients. In some embodiments, the 12-month CR rate is defined as the proportion of patients who have achieved a CR that lasts for at least 12 months.

In some embodiments, such treatments results in a 15-month CR rate in the population of patients of at least about 40%. In some embodiments, the 15-month CR rate in the population of patients is between about 43% to about 79%. In some embodiments, the 95% confidence interval of a 15-month CR rate in the population of patients is between about 43% to about 79%. In some embodiments, the 15-month CR rate in the population of patients is between about 40% to about 50%, about 50% to about 60%, about 60% to about 70%, about 70% to about 80%. In some embodiments, the 15-month CR rate in the population of patients is about 64%. In some embodiments, the 15-month CR rate is calculated from all treated patients in the population of patients. In some embodiments, the 15-month CR rate is defined as the proportion of patients who have achieved a CR that lasts for at least 15 months.

In some embodiments, provided herein is a method of treating high risk non-muscle invasive bladder cancer in a population of patients comprising administering gemcitabine locally to the bladder of each patient, wherein gemcitabine administration comprises delivering gemcitabine continuously to the bladder for at least 7 days during a gemcitabine delivery period, wherein such treatment results in a complete response rate that is at least 1.5 fold the complete response rate achieved by the standard of care. In some embodiments, such treatment results in a complete response rate that is between about 1.5 and 1.7, 1.7 and 1.9, 1.9 and 2.1, or 2.1 and 2.3 fold the complete response rate achieved by the standard of care, such treatment results in a complete response rate that is at least 1.5 fold the complete response rate achieved by the standard of care. In some embodiments, gemcitabine is delivered locally to the bladder about every three weeks (Q3W) for about 24 weeks. In some embodiments, gemcitabine is delivered locally to the bladder about every three weeks (Q3W) for about 24 weeks during an induction phase and then about every three months during a maintenance phase. In some embodiments, the therapy is a bladder sparing therapy. In some embodiments, a bladder sparing therapy is defined as a therapy that does not comprise the surgical removal of an individual's bladder and allows the induvial to retain their bladder. In some embodiments, the individual does not receive a radical cystectomy.

In some embodiments, such treatment results in a complete response rate that is between about 1.5 and 1.7, 1.7 and 1.9, 1.9 and 2.1, or 2.1 and 2.3 fold the complete response rate achieved by valrubicin. In some embodiments, such treatment results in a complete response rate that is between about 1.5 and 1.7, 1.7 and 1.9, 1.9 and 2.1, or 2.1 and 2.3 fold the complete response rate achieved by a checkpoint inhibitor therapy. In some embodiments, gemcitabine is delivered locally to the bladder about every three weeks (Q3W) for about 24 weeks. In some embodiments, gemcitabine is delivered locally to the bladder about every three weeks (Q3W) for about 24 weeks during an induction phase and then about every three months during a maintenance phase. In some embodiments, the therapy is a bladder sparing therapy. In some embodiments, the individual does not receive a radical cystectomy.

In some embodiments, such treatment results in a complete response rate that is between about 1.5 and 1.7, 1.7 and 1.9, 1.9 and 2.1, or 2.1 and 2.3 fold the complete response rate achieved by a checkpoint inhibitor therapy. In some embodiments, such treatment results in a complete response rate that is between about 1.5 and 1.7, 1.7 and 1.9, 1.9 and 2.1, or 2.1 and 2.3 fold the complete response rate achieved by pembrolizumab. In some embodiments, gemcitabine is delivered locally to the bladder about every three weeks (Q3W) for about 24 weeks. In some embodiments, gemcitabine is delivered locally to the bladder about every three weeks (Q3W) for about 24 weeks during an induction phase and then about every three months during a maintenance phase. In some embodiments, the therapy is a bladder sparing therapy. In some embodiments, the individual does not receive a radical cystectomy.

In some embodiments, such treatment results in a median duration of response (DOR) that is between about 9 and 18 months. In some embodiments, the median duration of response (DOR) is between about 9 and 30 months. In some embodiments, the median DOR is at least 9 months, at least 10 months, at least 11 months, at least 12 months, at least 13 months, or at least 14 months. In some embodiments, the median DOR is between about 10 and 11 months. In some embodiments, the median DOR is between about 25 and about 27 months. In some embodiments, the median DOR is about 11 months. In some embodiments, the median DOR is about 26 months. In some embodiments, a CR is ongoing after between about 9 and about 18 months of follow-up. In some embodiments, a CR is ongoing after at least 9 months, at least 10 months, at least 11 months, at least 12 months, at least 13 months, or at least 14 months of follow-up. In some embodiments, a CR is ongoing after a median of 10.6 months of follow-up. In some embodiments, the median DOR in the population of patients is not reached within 10.6 months. In some embodiments, a CR is ongoing after a median of 48 weeks of follow-up. In some embodiments, the median DOR in the population of patients is not reached within about 48 weeks. In some embodiments, the DOR measurement is calculated from patients who have responded to treatment. In some embodiments, DOR is defined as the date of first CR achieved to the date of first evidence of recurrence or progression or death, using cystoscopy, centrally read bladder biopsy and urine cytology, and imaging, if available.

In some embodiments, such treatment results in a 6-month duration of response (DOR) rate in the population of patients of at least about 50%. In some embodiments, the 6-month DOR rate in the population of patients is between about 69% to about 95%. In some embodiments, the 6-month DOR rate in the population of patients is between about 50% to about 55%, about 55% to about 60%, about 60% to about 65%, about 65% to about 70%, about 70% to about 75%, about 75% to about 80%, about 80% to about 85%, about 85% to about 90%, or about 90% to about 95%. In some embodiments, the 6-month DOR rate is about 87%. In some embodiments, a 6-month duration of response (DOR) rate is defined as the proportion of patients who have achieved a DOR that lasts for at least 6 months.

In some embodiments, such treatment results in a one-year duration of response (DOR) rate in the population of patients of at least about 50%. In some embodiments, the one-year DOR rate in the population of patients is between about 50% to about 89%. In some embodiments, the one-year DOR rate in the population of patients is between about 50% to about 55%, about 55% to about 60%, about 60% to about 65%, about 65% to about 70%, about 70% to about 75%, about 75% to about 80%, about 80% to about 85%, about 85% to about 89%. In some embodiments, the one-year DOR rate is about 77%. In some embodiments, the one-year DOR rate is about 75%. In some embodiments, a one-year duration of response (DOR) rate is defined as the proportion of patients who have achieved a DOR that lasts for at least one year.

In some embodiments, such treatment results in an 18-month duration of response (DOR) rate in the population of patients of at least about 50%. In some embodiments, the 18-month DOR rate in the population of patients is between about 50% to about 89%. In some embodiments, the 18-month DOR rate in the population of patients is between about 50% to about 55%, about 55% to about 60%, about 60% to about 65%, about 65% to about 70%, about 70% to about 75%, about 75% to about 80%, about 80% to about 85%, about 85% to about 89%. In some embodiments, the one-year DOR rate is about 75%. In some embodiments, an 18-month duration of response (DOR) rate is defined as the proportion of patients who have achieved a DOR that lasts for at least 18 months.

In some embodiments, such treatment results in a 6-month event-free survival in the population of patients of at least 85%. In some embodiments, such treatment results in a 6-month event-free survival in the population of patients of at least 67%. In some embodiments, such treatment results in a 6-month event-free survival in the population of patients of between about 85% and about 90%, about 90% and about 95%, or about 95% and about 100%. In some embodiments, such treatment results in a 6-month event-free survival in the population of patients of about 93%. In some embodiments, such treatment results in a 6-month event-free survival in the population of patients of about 87%. In some embodiments, such treatment results in a 6-month event-free survival in the population of patients of about 84%. In some embodiments, the 6-month event-free survival in the population of patients is between about 69% to about 95%. In some embodiments, the 95% confidence interval of a 6-month event-free survival in the population of patients is between about 69% to about 95%. In some embodiments, the 95% confidence interval of a 6-month event-free survival in the population of patients is between about 67% to about 93%. In some embodiments, such treatment results in a 12-month event-free survival in the population of patients of at least 70%. In some embodiments, such treatment results in a 12-month event-free survival in the population of patients of at least 44%. In some embodiments, such treatment results in a 12-month event-free survival in the population of patients of between about 70% and about 75%, about 75% and about 80%, about 80% and about 85%, about 85% and about 90%, about 90% and about 95%, or about 95% and about 100%. In some embodiments, such treatment results in a 12-month event-free survival in the population of patients of about 84%. In some embodiments, such treatment results in a 12-month event-free survival in the population of patients of about 67%. In some embodiments, such treatment results in a 12-month event-free survival in the population of patients of about 75%. In some embodiments, the 12-month event-free survival in the population of patients is between about 50% to about 88%. In some embodiments, the 95% confidence interval of a 12-month event-free survival in the population of is between about 50% to about 88%. In some embodiments, the 95% confidence interval of a 12-month event-free survival in the population of is between about 44% to about 83%. In some embodiments, such treatment results in an 18-month event-free survival in the population of patients of at least 70%. In some embodiments, such treatment results in an 18-month event-free survival in the population of patients of at least 44%. In some embodiments, such treatment results in an 18-month event-free survival in the population of patients of between about 70% and about 75%, about 75% and about 80%, about 80% and about 85%, about 85% and about 90%, about 90% and about 95%, or about 95% and about 100%. In some embodiments, such treatment results in an 18-month event-free survival in the population of patients of about 84%. In some embodiments, such treatment results in an 18-month event-free survival in the population of patients of about 67%. In some embodiments, such treatment results in an 18-month event-free survival in the population of patients of about 75%. In some embodiments, the 18-month event-free survival in the population of patients is between about 50% to about 88%. In some embodiments, the 95% confidence interval of an 18-month event-free survival in the population of patients is between about 50% to about 88%. In some embodiments, the 95% confidence interval of a 18-month event-free survival in the population of is between about 44% to about 83%. In some embodiments, such treatment results in a 24-month event-free survival in the population of patients of at least 70%. In some embodiments, such treatment results in a 24-month event-free survival in the population of patients of at least 44%. In some embodiments, such treatment results in a 24-month event-free survival in the population of patients of between about 70% and about 75%, about 75% and about 80%, about 80% and about 85%, about 85% and about 90%, about 90% and about 95%, or about 95% and about 100%. In some embodiments, such treatment results in a 24-month event-free survival in the population of patients of about 75%. In some embodiments, such treatment results in a 24-month event-free survival in the population of patients of about 67%. In some embodiments, the 24-month event-free survival in the population of patients is between about 50% to about 88%. In some embodiments, the 95% confidence interval of a 24-month event-free survival in the population of patients is between about 50% to about 88%. In some embodiments, the 95% confidence interval of a 24-month event-free survival in the population of is between about 44% to about 83%. In some embodiments, event-free survival is defined as the time between treatment and having a specific ‘event’ such as a symptom recurrence, progression, or death. In some embodiments, a 6-month event-free survival is defined as the time between treatment and having a specific ‘event’ such as a symptom recurrence, progression, or death over a 6-month period. In some embodiments, a 12-month event-free survival is defined as the time between treatment and having a specific ‘event’ such as a symptom recurrence, progression, or death over a 12-month period. In some embodiments, an 18-month event-free survival is defined as the time between treatment and having a specific ‘event’ such as a symptom recurrence, progression, or death over an 18-month period. In some embodiments, a 24-month event-free survival is defined as the time between treatment and having a specific ‘event’ such as a symptom recurrence, progression, or death over a 24-month period.

In some embodiments, there are at least 20 individuals in the population of patients. In some embodiments, there are between about 20 and about 30, about 30 and about 40, about 40 and about 50, about 50 and about 60, about 60 and about 70, about 70 and about 80, about 80 and about 90, or about 90 and about 100 individuals in the population of patients. In some embodiments, there are 23 individuals in the population of patients. In some embodiments, there are 54 individuals in the population of patients.

In some embodiments, provided herein is a method of treating high-risk non-muscle invasive bladder cancer in a population of patients comprising administering gemcitabine locally to the bladder of each patient, wherein gemcitabine administration comprises delivering gemcitabine continuously to the bladder for at least 7 days during a gemcitabine delivery period, wherein a two-year surveillance period begins at about 24 to 47 days after the first administration of gemcitabine. In some embodiments, cystoscopy and cytology assessments are performed at about week 12, about week 24, about week 36, about week 48, about week 60, about week 72, about week 84, about week 96, about week 120, and about week 144 during treatment. In some embodiments, a complete response is observed during a scheduled cystoscopy and cytology assessment during treatment. In some embodiments, gemcitabine is delivered locally to the bladder about every three weeks (Q3W) for about 24 weeks. In some embodiments, gemcitabine is delivered locally to the bladder about every three weeks (Q3W) for about 24 weeks during an induction phase and then about every three months during a maintenance phase. In some embodiments, the therapy is a bladder sparing therapy. In some embodiments, the individual does not receive a radical cystectomy.

In some embodiments, provided herein is a method of treating high-risk non-muscle invasive bladder cancer that is unresponsive to intravesical Bacillus Calmette-Guerin (BCG) therapy in a population of patients comprising administering gemcitabine locally to the bladder of each patient, wherein gemcitabine administration comprises delivering gemcitabine continuously to the bladder for at least 7 days during a gemcitabine delivery period, wherein such treatment results in an improved complete response rate compared to a standard of care treatment. In some embodiments, the standard of care treatment comprises radical cystectomy. In some embodiments, the standard of care treatment comprises a systemic or intravesical chemotherapy. In some embodiments, the standard of care treatment comprises mitomycin C therapy. In some embodiments, the standard of care treatment comprises Valrubicin. In some embodiments, the standard of care treatment comprises pembrolizumab. In some embodiments, the standard of care treatment comprises paclitaxel. In some embodiments, the standard of care treatment comprises an immune checkpoint therapy such as pembrolizumab. In some embodiments, the standard of care treatment includes an adjuvant chemotherapy such as mitomycin C, epirubicin, or doxorubicin. In some embodiments, the standard of care treatment comprises a systemic or intravesical combination chemotherapy. In some embodiments, the combination intravesical chemotherapy is composed of gemcitabine and docetaxel. In some embodiments, gemcitabine is delivered locally to the bladder about every three weeks (Q3W) for about 24 weeks. In some embodiments, gemcitabine is delivered locally to the bladder about every three weeks (Q3W) for about 24 weeks during an induction phase and then about every three months during a maintenance phase. In some embodiments, the therapy is a bladder sparing therapy. In some embodiments, the individual does not receive a radical cystectomy.

In some embodiments, provided herein is a method of treating high-risk non-muscle invasive bladder cancer comprising administering gemcitabine to an individual, wherein gemcitabine administration comprises delivering gemcitabine continuously to the bladder for at least 7 days during a gemcitabine delivery period, wherein following such treatment the individual exhibits no evidence of bladder cancer. In some embodiments, following such treatment, the individual has a biopsy-proven benign or low-grade NMIBC. In some embodiments, the biopsy-proven benign or low-grade NMIBC comprises a low-grade Ta bladder cancer. In some embodiments, the low-grade NMIBC is a non-invasive papillary (Ta) tumor. In some embodiments, gemcitabine is delivered locally to the bladder about every three weeks (Q3W) for about 24 weeks. In some embodiments, gemcitabine is delivered locally to the bladder about every three weeks (Q3W) for about 24 weeks during an induction phase and then about every three months during a maintenance phase. In some embodiments, the therapy is a bladder sparing therapy. In some embodiments, the individual does not receive a radical cystectomy.

In some embodiments, the methods provided herein result in bladder sparing in an individual with HR-NIMBC that is unresponsive to BCG therapy. In some embodiments, the bladder sparing methods provided herein result in significant improvements on quality of life for treated individuals. In some embodiments, a quality-of-life score is calculated for treated individuals based on results from the European Organization for Research and Treatment of Cancer-Quality of Life Questionnaire-Core (EORTC-QLQ-C30) or European Organization for Research and Treatment of Cancer-Non-Muscle Invasive Bladder Cancer Questionnaire (EORTC-QLQ-NMIBC24). In some embodiments, the quality-of-life score of the individual is maintained or increases following treatment by the methods provided herein. In some embodiments, quality of life improvements include avoiding the risks of mortality and morbidity associated with radical cystectomy, as well as maintaining continence, sexual function, fertility, and bowel function. In some embodiments, gemcitabine is delivered locally to the bladder about every three weeks (Q3W) for about 24 weeks. In some embodiments, gemcitabine is delivered locally to the bladder about every three weeks (Q3W) for about 24 weeks during an induction phase and then about every three months during a maintenance phase. In some embodiments, the individual does not receive a radical cystectomy.

In some embodiments, the EORTC-QLQ-C30 is a core questionnaire for evaluating the quality of life of participants participating in cancer clinical studies. In some embodiments, the EORTC-QLQ-C30 is a 30-item questionnaire with 9 multi-item subscales and 6 single items. In some embodiments, the EORTC-QLQ-C30 incorporates 5 functional scales (physical, role, cognitive, emotional, and social functioning), 3 symptom scales (fatigue, pain, and nausea or vomiting), and a global health status or quality of life scale. In some embodiments, the single items assess dyspnea, appetite loss, sleep disturbance, constipation, diarrhea, and perceived financial impact of disease and treatment. Ratings for each item range from 1 (not at all) to 4 (very much). In some embodiments, the EORTC-QLQ-C30 is designed to be used across cancer populations and takes about 11 minutes to complete.

In some embodiments, the EORTC-QLQ-NMIBC24 is a 24-item questionnaire for evaluating the quality of life of participants with superficial (non-muscle invasive) bladder cancer. In some embodiments, the questionnaire is designed to supplement the QLQ-C30 and incorporates 6 multi-item scales and 5 single items. In some embodiments, ratings for each item in the EORTC-QLQ-NMIBC24 range from 1 (not at all) to 4 (very much). In some embodiments, the scales cover urinary symptoms, malaise, worries about the future, bloating and flatulence, sexual function, and male sexual problems. In some embodiments, the single items assess intravesical treatment issues, sexual intimacy, sexual enjoyment, risk of contaminating partner, and female sexual problems. In some embodiments, the EORTC-QLQ-NMIBC24 takes about 8 minutes to complete.

### II. Intravesical Drug Delivery Systems

The various aspects and embodiments described in this section in the context of a method of treatment comprising administering gemcitabine using an intravesical drug delivery system also apply to gemcitabine for use according to the methods described herein, and minitablets comprising gemcitabine for use according to the methods described herein. In particular, the aspects and embodiments described in this section also relate to gemcitabine for use with an intravesical drug delivery system for use in the methods provided herein, wherein the method comprises continuous delivery of gemcitabine to the bladder by an intravesical drug delivery system comprising the intravesical device, and to gemcitabine for use according to the methods described herein, wherein the gemcitabine is administered by an intravesical drug delivery system.

In some embodiments, the methods provided herein include administering gemcitabine using an intravesical drug delivery system. Any intravesical drug delivery system comprises an intravesical device and gemcitabine. In some embodiments, the intravesical device is capable of delivering the gemcitabine within the bladder in suitable amounts, at suitable rates, and over suitable durations.

In some embodiments, the intravesical device is configured to be deployed into the bladder through the working channel of a catheter, cystoscope, or other deployment instrument positioned in the urethra. In some embodiments, the intravesical device is elastically deformable between a deployment shape for passage through the deployment instrument and a bladder retention shape in which the device (i) resists becoming entrained in urine and excreted when the individual voids, and (ii) intended to be tolerable to the individual, e.g., does not overly press on the bladder wall, or enter the ureter orifices. Such intravesical devices are known in the art. In some embodiments, the intravesical drug delivery system or intravesical device is one described in one or more of U.S. Pat. Nos. 10,543,166, 9,283,361, 8,679,094, 10,857,336, 9,457,176, 8,690,840, 9,107,816, 10,137,287, 9,814,671, 10,729,823, 10,315,019, 10,933,170, 10,737,078, 10,894,150, and 11,020,575, which are incorporated herein by reference in their entirety.

In various embodiments, the intravesical drug delivery system may release the gemcitabine continuously or intermittently to achieve a concentration of the drug in the bladder that produces a sustained, therapeutically effective concentration of the drug in urine in the bladder as described in the methods provided herein. In certain embodiments, the intravesical drug delivery system may release the gemcitabine in an amount of about 0.5 mg/day to about 50 mg/day, about 1 mg/day to about 40 mg/day, about 3 mg/day to about 30 mg/day, about 5 mg/day to about 20 mg/day, or about 1 mg/day to about 15 mg/day. In certain embodiments, the intravesical drug delivery system may release the gemcitabine in an amount (such as a maximum amount) of about 42 mg/day. In certain embodiments, the intravesical drug delivery system may release the gemcitabine in an amount of about 12 mg/day. In certain embodiments, these release rates are provided over a treatment period as described herein. In certain embodiments, these release rates are provided over a delivery period from 14 days to 21 days. In certain embodiments, these release rates are provided over a delivery period of about 7 days. In certain embodiments, these release rates are provided over a delivery period of about 14 days. In some embodiments, the intravesical drug delivery system releases gemcitabine at a maximum rate of 42 mg FBE/day on day two and a rate of 12 mg FBE/day on day 7.

In some embodiments, the intravesical device include a device body, which may be a housing, and a drug payload which includes a suitable amount of the gemcitabine. The drug payload may be referred to herein as a dosage form. The drug payload may be contained in a lumen, or reservoir, within the device body. The device body, in combination with the form and formulation of the drug payload, may control release/delivery of the gemcitabine into the bladder. The release of gemcitabine from the intravesical drug delivery systems described herein may be driven and controlled by different mechanisms of action. In various embodiments, the gemcitabine may be released from the intravesical device by diffusion through a wall of the housing, by diffusion through one or more defined apertures in a wall of the housing, by osmotic pressure through an aperture in the housing, by osmotic pressure through one or more transiently formed microchannels in the housing, by erosion of a drug formulation in contact with urine in the bladder, by diffusion through a drug-permeable polymer or matrix component defining part of the device housing, or by a combination thereof. Examples of such intravesical devices or intravesical drug delivery systems are described in the above-listed U.S. patents incorporated by reference.

Following in vivo deployment, the intravesical drug delivery system releases the gemcitabine. Release may occur, as described above, due to an osmotic pressure gradient between the interior and exterior of the intravesical device, the drug passing through one or more orifices or passing pores in the intravesical device under the force of osmotic pressure. Release may also occur by diffusion, whereby the drug passes through one or more orifices or passing pores in the intravesical device and/or through a drug-permeable wall of the intravesical device, due to a drug concentration gradient between the interior and exterior of the intravesical device. Combinations of these release modes within a single intravesical device are possible, and in some embodiments are preferred in order to achieve an overall drug release profile not readily achievable from either mode individually.

In some embodiments, at least some of the material(s) used to form the device body may be water permeable so that solubilizing fluid (e.g., urine) can diffuse into the intravesical device to contact the drug payload, e.g., enter a drug reservoir portion to solubilize the non-liquid forms of the gemcitabine (and/or any immunomodulating agent, additional therapeutic agent, functional agent, or a combination thereof also) contained within the intravesical device following its deployment into the bladder. For example, silicone, certain thermoplastic polyurethanes, or other biocompatible elastomeric materials may be used. In other embodiments, the device body may be formed, at least in part, of a water-impermeable material.

The drug payload may be housed in the intravesical device in various forms, which may depend on the particular mechanism by which the intravesical drug delivery system controllably releases the gemcitabine into fluid (e.g., urine) in the bladder. In some embodiments, the drug is provided in a solid, semi-solid, or other non-liquid form, which advantageously may facilitate stable storage of the drug before the intravesical device is used and advantageously may enable the drug payload of the intravesical device to be stored in smaller volume than would be possible if the drug were housed in the form of a liquid solution. In some embodiments, the non-liquid form is selected from tablets, granules, pellets, powders, semisolids (e.g., an ointment, cream, paste, or gel), capsules, and combinations thereof. In some embodiments, the intravesical device body includes a drug reservoir lumen. In some of these embodiments, the drug reservoir lumen holds one or several drug tablets or other solid drug units, wherein at least a portion of the tablets/units includes gemcitabine. In some embodiments, the intravesical device holds from about 10 to 100 cylindrical drug tablets, such as mini-tablets. In certain embodiments, the mini-tablets each have a diameter of about 1.0 to about 3.3 mm, such as about 1.5 to about 3.1 mm, and a length of about 1.5 to about 4.7 mm, such as about 2.0 to about 4.5 mm. In one embodiment, the drug is in the form of a plurality of tablets, such as mini-tablets described in U.S. Pat. No. 8,343,516, which is incorporated by reference herein.

In some other embodiments, the gemcitabine may be provided in the intravesical device as suspensions, solutions, or emulsions in oily or aqueous vehicles, and may contain formulatory agents such as suspending, stabilizing and/or dispersing agents. Alternatively, the gemcitabine may be in powder form, obtained by aseptic isolation of sterile solid or by lyophilization from solution, for constitution with a suitable vehicle, e.g., sterile, pyrogen-free water, before use.

In some particular embodiments, the intravesical drug delivery system is configured to provide controlled release of the gemcitabine by osmotic pressure, as described, for example, in U.S. Pat. No. 10,729,823, which as noted above is incorporated by reference herein. In some of these embodiments, the intravesical device includes a housing defining a reservoir; a first unit (e.g., a first plurality of tablets) contained within the reservoir, the first unit comprising gemcitabine; and a second unit (e.g., a second plurality of tablets) contained within the reservoir, the second unit comprising a functional agent and not comprising gemcitabine. One or more of the first unit tablets may fill a length from about 1 cm to about 5 cm of the lumen of the tube, and one or more of the second unit tablets may fill a length from about 10 cm to about 14 cm of the lumen of the tube. The functional agent is a substance that facilitates in vivo release of the drug from the housing. For example, it may be an osmotic agent, such as urea. In some embodiments, the housing is in the form of an elongated elastomeric tube having a lumen (i.e., the reservoir) in which all of the first and second tablets are aligned and contained, wherein the elastomeric tube includes one or more apertures or microchannels configured to provide release of the gemcitabine in vivo by osmotic pressure.

In some embodiments, the intravesical drug delivery system contains a unit concentration of about 225 mg of gemcitabine. In some of these embodiments, the intravesical drug delivery system is configured to deliver about 100 to about 225 mg of gemcitabine (e.g., about 140 mg, about 160 mg, about 180 mg, about 200 mg, or about 220 mg of gemcitabine) to the individual over at least about or about a seven day period, at least about or about a fourteen-day period, or at least about or about a three-week period.

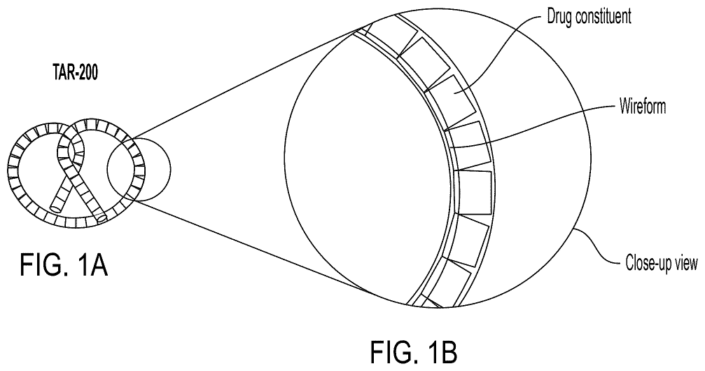

In some embodiments, the intravesical drug delivery system is a TAR-200/gemcitabine product (hereafter, TAR-200), an intravesical drug delivery system consisting of two components: (i) the drug constituent, which consists of gemcitabine minitablets (225 mg, free base equivalent) and osmotic minitablets containing urea as the osmotic agent; and (ii) the intravesical device constituent, which is comprised of a dual lumen silicone part with a single laser-machined orifice and a superelastic nitinol wire (as shown in FIG. 1). The large lumen of the silicone part contains the gemcitabine and urea minitablets and serves as an osmotic pump to release drug in a controlled manner. The smaller lumen contains a nitinol wire in a predefined coil form to provide retention of the intravesical drug delivery system in the individual's bladder during the indwelling period.

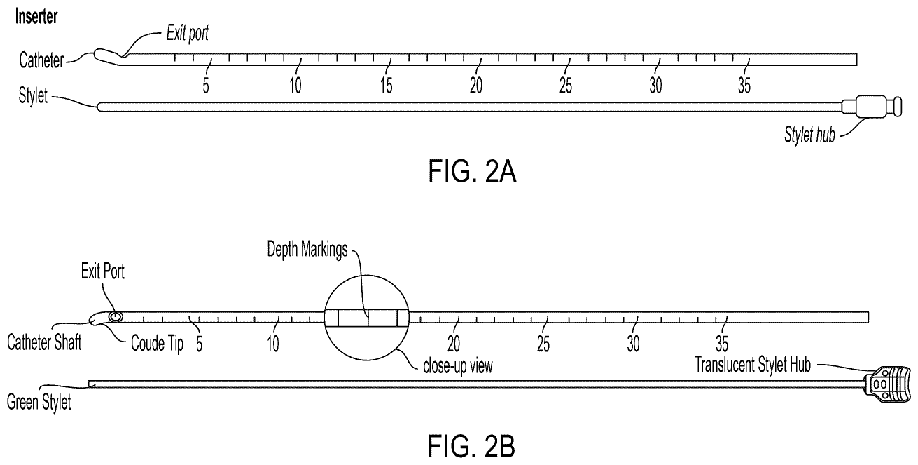

In some embodiments, the intravesical drug delivery system is provided in a kit with a urological placement catheter configured to facilitate deployment of the intravesical device into the bladder of an individual. In some embodiments, the urological placement catheter is described in U.S. Pat. No. 10,064,980, which is incorporated herein by reference. In some embodiments, TAR-200 is kitted with a urological placement catheter (Urinary Placement Catheter), for the transurethral placement of intravesical drug delivery systems, as shown in FIG. 2. In some embodiments, TAR-200 is removed from the bladder transurethrally via cystoscopy and endoscopic graspers.

III. Antibodies that Bind to PD-1

The various aspects and embodiments described in this section in the context of a method of treatment comprising administering to an individual an antibody or an antigen binding fragment thereof that binds to PD-1 also apply to the antibody or antigen binding fragment thereof that binds to PD-1 for use according to the methods described herein.

The following section describes various aspects (embodiments) of PD-1-antibodies, any and all of which can be used in the methods described herein.

In some aspects, provided herein are methods for treating bladder cancer comprising administering to an individual an antibody or an antigen binding fragment thereof that binds to PD-1. “PD-1” refers to human programmed cell death protein 1, PD-1. PD-1 is also known as CD279 or PDCD1. The amino acid sequence of the mature human PD-1 (without signal sequence) is shown in SEQ ID NO: 11. The extracellular domain spans residues 1-150, the transmembrane domain spans residues 151-171 and the cytoplasmic domain spans residues 172-268 of SEQ ID NO: 11.

SEQ ID NO: 11
PGWFLDSPDRPWNPPTFSPALLVVTEGDNATFTCSFSNTSESFVLNWYR
MSPSNQTDKLAAFPEDRSQPGQDCRFRVTQLPNGRDFHMSVVRARRNDS
GTYLCGAISLAPKAQIKESLRAELRVTERRAEVPTAHPSPSPRPAGQFQ
TLVVGVVGGLLGSLVLLVWVLAVICSRAARGTIGARRTGQPLKEDPSAV
PVFSVDYGELDFQWREKTPEPPVPCVPEQTEYATIVFPSGMGTSSPARR
GSADGPRSAQPLRPEDGHCSWPL

In some embodiments, the antibody that binds to PD-1 comprises amino acid sequences set forth in the Table 1 and any of those disclosed in U.S. Pat. No. 10,894,830 (herein incorporated by reference in its entirety).

TABLE 1
PD-1 Antibody Amino Acid Sequences

| | | |
|---|---|---|
| HCDR1 | SEQ ID | SYAIS |
| | NO: 1 | |
| HCDR2 | SEQ ID | GHPIFDTANYAQKFQG |
| | NO: 2 | |
| HCDR3 | SEQ ID | PGLAAAYDTGSLDY |
| | NO: 3 | |
| LCDR1 | SEQ ID | RASQSVRSYLA |
| | NO: 4 | |
| LCDR2 | SEQ ID | DASNRAT |
| | NO: 5 | |
| LCDR3 | SEQ ID | QQRNYWPLT |
| | NO: 6 | |
| heavy | SEQ ID | QVQLVQSGAEVKKPGSSVKVSCKASGGTFSSYAISWVRQAPGQGLEWMGG |
| chain | NO: 7 | HIPIFDTANYAQKFQGRVTITADESTSTAYMELSSLRSEDTAVYYCARPG |
| variable | | LAAAYDTGSLDYWGQGTLVTVSS |
| region | | |
| (VH) | | |
| light chain | SEQ ID | EIVLTQSPATLSLSPGERATLSCRASQSVRSYLAWYQQKPGQAPRLLIYD |
| variable | NO: 8 | ASNRATGIPARFSGSGSGTDFTLTISSLEPEDFAVYYCQQRNYWPLTFGQ |
| region | | GTKVEIK |
| (VL) | | |
| heavy | SEQ ID | QVQLVQSGAEVKKPGSSVKVSCKASGGTFSSYAISWVRQAPGQGLEWMGG |
| chain | NO: 9 | IIPIFDTANYAQKFQGRVTITADESTSTAYMELSSLRSEDTAVYYCARPG |
| | | LAAAYDTGSLDYWGQGTLVTVSSASTKGPSVFPLAPCSRSTSESTAALGC |
| | | LVKDYFPEPVTVSWNSGALTSGVHTFPAVLQSSGLYSLSSVVTVPSSSLG |
| | | TKTYTCNVDHKPSNTKVDKRVESKYGPPCPPCPAPEFLGGPSVFLFPPKP |
| | | KDTLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPV |
| | | LDSDGSFFLYSRLTVDKSRWQEGNVFSCSVMHEALHNHYTQKSLSLSLG |
| light | SEQ ID | EIVLTQSPATLSLSPGERATLSCRASQSVRSYLAWYQQKPGQAPRLLIYD |
| chain | NO: 10 | ASNRATGIPARFSGSGSGTDFTLTISSLEPEDFAVYYCQQRNYWPLTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |

In some embodiments, the antibody that binds to PD-1 comprises a variable heavy chain (VH) and a variable light chain (VL), wherein the VH comprises a CDRH1 comprising the amino acid sequence set forth in SEQ ID NO:1, a CDRH2 comprising the amino acid sequence set forth in SEQ ID NO:2, a CDRH3 comprising the amino acid sequence set forth in SEQ ID NO:3, and the VL comprises a CDRL1 comprising the amino acid sequence set forth in SEQ ID NO: 4, a CDRL2 comprising the amino acid sequence set forth in SEQ ID NO: 5, and a CDRL3 comprising the amino acid sequence set forth in SEQ ID NO: 6. In some embodiments, the antibody that binds to PD-1 is an antagonistic anti-PD-1 antibody.

In some embodiments, the antagonistic anti-PD-1 antibody is cetrelimab. Cetrelimab is an IgG4/K antibody characterized by the following amino acid sequences: the CDRH1 of SEQ ID NO: 1, the CDRH2 of SEQ ID NO: 2, the CDRH3 of SEQ ID NO: 3, the CDRL1 of SEQ ID NO: 4, the CDRL2 of SEQ ID NO: 5, the CDRL3 of SEQ ID NO: 6, the VH of SEQ ID NO: 7, the VL of SEQ ID NO: 8, the heavy chain of SEQ ID NO: 9 and the light of SEQ ID NO: 10.

In some embodiments, the antibody or antigen binding fragment thereof that binds to PD-1 is administered at a dose of between about 240 mg and about 480 mg. In some embodiments, the antibody or antigen binding fragment thereof that binds to PD-1 is administered at a dose of about 360 mg.

In some embodiments, the antagonistic anti-PD-1 antibody or the antigen binding fragment thereof is administered or provided for administration in a pharmaceutical composition comprising between about 10 mg/ml to about 150 mg/ml (e.g., 30 mg/ml, e.g., 150 mg/ml) of the antagonistic anti-PD-1 antibody or the antigen binding fragment thereof and one or more pharmaceutically acceptable excipients.

“Pharmaceutically acceptable excipients” refer to solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

In some embodiments, the anti-PD-1 antibody or the antigen binding fragment thereof is administered by an intravenous infusion.

In some embodiments, the antagonistic anti-PD-1 antibody or the antigen binding fragment thereof is diluted into a volume of between about 100 ml and 1000 ml prior to administration.

In some embodiments, duration of the intravenous infusion is between about 20 minutes and about 80 minutes.

In some embodiments, duration of the intravenous infusion is about 20, about 30, about 40, about 50, about 60, about 70 or about 80 minutes.

In some embodiments, the anti-PD-1 antibody or the antigen binding fragment thereof is an IgG1, an IgG2, and IgG3 or an IgG4 isotype. In some embodiments, the anti-PD-1 antibody or the antigen binding fragment thereof is an IgG4 isotype. In some embodiments, the antagonistic anti-PD-1 antibody or the antigen binding fragment thereof is an IgG4 isotype and comprises proline at position 228, residue numbering according to the EU Index. In some embodiments, the antagonistic anti-PD-1 antibody is a nG4m (a) allotype. In some embodiments, the antagonistic anti-PD-1 antibody or an antigen binding fragment thereof has at least one substitution in an Fc region to modulate antibody effector functions or antibody half-life.

### IV. Articles of Manufacture

In some aspects, provided herein is an article of manufacture comprising a packaging material and an intravesical drug delivery system comprising 225 mg gemcitabine and a package insert comprising instructions for use according to the methods disclosed herein. In some embodiments, the article of manufacture further comprises a urinary placement catheter. In some embodiments, the urinary placement catheter comprises a catheter and a stylet. In some embodiments, the article of manufacture further comprises a lubricant and/or a syringe. In some embodiments, the instructions for use provide instructions for administering the intravesical drug delivery system comprising 225 mg gemcitabine according to the methods provided herein.

In some embodiments, the article of manufacture comprises an intravesical drug delivery system comprising about 150 mg to about 300 mg, about 200 mg to about 250 mg, or about 225 mg gemcitabine. In some embodiments, the article of manufacture further comprises a urinary placement catheter. In some embodiments, the urinary placement catheter comprises a catheter and a stylet. In some embodiments, the article of manufacture further comprises a lubricant and/or a syringe. In some embodiments, the instructions for use provide instructions for administering the intravesical drug delivery system comprising 225 mg gemcitabine according to the methods provided herein.

In some embodiments, the article of manufacture comprises a packaging material, a TAR-200 intravesical drug delivery system as shown in FIGS. 1A-1B and a urinary placement catheter comprising a catheter and a stylet as shown in FIGS. 2A-2B. TAR-200 is an intravesical drug delivery system consisting of two components: (i) the drug constituent, which consists of gemcitabine minitablets (225 mg, free base equivalent) and osmotic minitablets containing urea as the osmotic agent; and (ii) the intravesical device constituent, which is comprised of a dual lumen silicone part with a single laser-machined orifice and a superelastic nitinol wire (“wireform”). The large lumen of the silicone part contains the gemcitabine and urea minitablets and serves as an elementary osmotic pump to release drug in a controlled manner. The smaller lumen contains the nitinol wire in a predefined form to provide retention of the system in the bladder during the indwelling period. In some embodiments, the intravesical device comprises a deployment shape. In some embodiments, the intravesical drug delivery system and the urinary placement catheter are packaged in separate pouches (e.g., Tyvek pouches). In some embodiments, the intravesical drug delivery system and the urinary placement catheter are packaged together in the same pouch (e.g., a Tyvek pouch).

In some embodiments, the package insert provides instructions for insertion and/or removal of the intravesical drug delivery system. In some embodiments, the package insert provides instructions for the intravesical drug delivery system to be inserted transurethrally into the bladder using the Urinary Placement Catheter and removed from the bladder using endoscopic non-cutting grasping forceps and a standard flexible or rigid cystoscope. In some embodiments, the package insert provides instructions for inserting the intravesical drug delivery system, comprising: (1) Prepare for Placement: gather supplies, including Inserter, TAR-200, and two 10 mL slip tip syringes filled with water-based lubricant. If needed, fill syringes manually. Open TAR-200 outer foil pouch at tear notch. Open TAR-200 and Inserter Tyvek pouches from chevron side; (2) Introduce Empty Catheter into Bladder: (a) Lubricate tip of empty Catheter; (b) Introduce empty Catheter (without stylet) into urethra and advance until urine return. Note depth marking printed on Catheter to maintain position during procedure; (3) After Placing Catheter into Bladder, Inject Lubricant and Insert TAR-200: (a) Using first lubricant syringe, inject 2-3 mL of lubricant into Catheter's end; (b) Load either end of TAR-200 into Catheter and advance until completely uncoiled inside Catheter; (c) Using second lubricant syringe, inject another 2-3 mL of lubricant into Catheter's end; (d) Slowly insert stylet completely into Catheter until stylet hub is in flush contact with Catheter's end. This will ensure TAR-200 completely exits Catheter into bladder; I Remove Catheter and stylet from urethra together. TAR-200 should remain inside bladder. Note: If TAR-200 cannot be advanced due to resistance, remove catheter and stylet completely. Ensure TAR-200 is removed as well. Do not attempt to place the removed TAR-200 again.

In some embodiments, the package insert provides instructions for inserting the intravesical drug delivery system, comprising: (1) Gather supplies, including the UPC and two sterile 10 ml slip tip syringes filled with sterile water-based lubricant. If needed, fill syringes manually; (2) Inspect all UPC device pouches for damage. DO NOT USE any product that is damaged or has damaged packaging; (3) After preparation of the urethral meatus, lubricate the tip of the empty catheter shaft. NOTE: Depth markings are aligned with upward direction of coude tip; (4) Introduce empty catheter shaft (without green stylet) into urethra and advance until urine return. Note depth marking printed on catheter shaft to maintain position during procedure; (5) Refer to the instructions for use provided with the intravesical drug delivery system on how to load the intravesical drug delivery system into the UPC; (6) Once the intravesical drug delivery system is loaded into the UPC, slowly insert the green stylet until the white stylet hub is in flush contact with the catheter's end. This will ensure that the intravesical drug delivery system completely exits the UPC into the bladder; (7) After withdrawing the catheter shaft, visually confirm that the intravesical drug delivery system is no longer in the UPC.

In some embodiments, the package insert provides instructions for removing the intravesical drug delivery system, comprising: Insert Cystoscope and Grasp TAR-200: (a) Introduce cystoscope into bladder and visualize TAR-200, (b) Introduce grasping forceps through cystoscope's working channel, (c) Grasp TAR-200 over silicone tubing and wireform. Do not grasp on or near ends of TAR-200; and Remove TAR-200: (a) While grasping TAR-200, remove cystoscope and forceps out of urethra together to remove TAR-200 under direct vision. Do not remove TAR-200 through cystoscope's working channel. Doing so can damage TAR-200 and/or cystoscope; (b) After removal, inspect TAR-200 to confirm it is intact and unbroken.

In some embodiments, the package insert provides instructions for dosing TAR-200 (225 mg gemcitabine) in a 21 day dosing cycle, wherein a TAR-200 intravesical drug delivery system is inserted transurethrally into the bladder, remains indwelling for three weeks, and is then removed and replaced with a new TAR-200 intravesical drug delivery system every 3 weeks for the duration of the dosing period. In some embodiments, insertion and/or removal of the TAR-200 intravesical drug delivery system is inserted every 3 weeks±3 days.

In some embodiments, the package insert provides instructions for a method of treating individuals with high-risk non-muscle invasive bladder cancer comprising administering gemcitabine locally to the bladder of the individual, wherein each gemcitabine administration comprises delivering gemcitabine continuously to the bladder for at least 7 days during a gemcitabine delivery period. In some embodiments, gemcitabine is administered to the bladder about every three weeks for up to the first about six months and about every three months thereafter. In some embodiments, gemcitabine is administered to the bladder about every three weeks for about the first 24 weeks and then about every three months thereafter for a total of about two years. In some embodiments, about 225 mg of gemcitabine is administered during each administration. In some embodiments, the individual has BCG unresponsive non-muscle invasive bladder cancer. In some embodiments, the individual has refused or is ineligible for radical cystectomy.

In some aspects, provided herein is an article of manufacture comprising a packaging material and an antibody that binds to PD-1 and a package insert comprising instructions for use according to the methods disclosed herein. In some embodiments, the antibody that binds to PD-1 is contained in a vial or a syringe. In some embodiments, the antibody is cetrelimab. In some embodiments, the antibody is packaged as a lyophilized formulation in a vial. In some embodiments, the antibody is packaged as a liquid formulation in a vial. In some embodiments, the cetrelimab is provided in a pharmaceutical composition suitable for intravenous administration, such as any one of the pharmaceutical compositions described herein. In some embodiments, the instructions for use comprise instructions for administering an antibody that binds to PD-1. In some embodiments, the PD-1 antibody is in a vial. In some embodiments, the PD-1 antibody is packaged with an infusion pump and an intravenous administration set (IV set).

In some embodiments, the article of manufacture comprises an antibody that binds to PD-1. In some embodiments, the antibody is cetrelimab. In some embodiments, the antibody is packaged as a lyophilized formulation in a vial. In some embodiments, the antibody is packaged as a liquid formulation in a vial. In some embodiments, the cetrelimab is provided in a pharmaceutical composition suitable for intravenous administration, such as any one of the pharmaceutical compositions described herein. In some embodiments, the instructions for use provide instructions for administering the antibody that binds to PD-1 according to the methods provided herein. In some embodiments, the PD-1 antibody is in a vial. In some embodiments, the PD-1 antibody is packaged with an infusion pump and an intravenous administration set (IV set).

In some embodiments, the article of manufacture comprises Cetrelimab 240 mg Final Lyophilized Product. In some embodiments, the package insert provides instructions for reconstitution of the 240 mg/vial in 30 mL solution.

In some embodiments, the package insert provides instructions for intravenous administration of the anti-PD-1 antibody. In some embodiments, the package insert provides instructions for further dilution of the reconstituted product with 0.9% sodium chloride injection to make 100 mL intravenous (IV) solution, and for administration of cetrelimab via IV infusion.

In some embodiments, the package insert provides instructions for dosing cetrelimab in a 21 day dosing cycle, wherein cetrelimab is administered intravenously about every three weeks for the duration of the dosing period. In some embodiments, administration of cetrelimab is instructed every 3 weeks±3 days. In some embodiments, the package insert provides instructions for administration of cetrelimab.

In some embodiments, the package insert provides instructions for a method of treating individuals with high-risk non-muscle invasive bladder cancer comprising administering to the individual an antibody that binds to PD-1 about every three weeks for the duration of the dosing period. In some embodiments, the antibody that binds to PD-1 is administered about every three weeks for about the first 18 months. In some embodiments, about 360 mg of the PD-1 antibody is administered (e.g., via intravenous infusion) during each administration. In some embodiments, the individual has BCG unresponsive non-muscle invasive bladder cancer. In some embodiments, the individual has refused or is ineligible for radical cystectomy.

### Kits

In some aspects, provided herein are kits for carrying out any methods described herein.

In some embodiments, provided herein is a kit comprising an intravesical drug delivery system containing 225-mg free base equivalents of gemcitabine, such as the TAR-200 device illustrated in FIGS. 1A-1B. In some embodiments, the kit further comprises a Urinary Placement Catheter (“Inserter”) consisting of a clear catheter and a colored or opaque stylet (e.g., a green stylet), such as the catheter and stylet as shown in FIGS. 2A-2B. In some embodiments, the intravesical drug delivery system comprises a deployment shape. In some embodiments, the intravesical drug delivery system and the urinary placement catheter are packaged in separate pouches (e.g., Tyvek pouches). In some embodiments, the intravesical drug delivery system and the urinary placement catheter are packaged together in the same pouch (e.g., a Tyvek pouch). In some embodiments, the kit further comprises instructions for inserting the intravesical drug delivery system into the bladder of an individual, and/or instructions for removing the intravesical drug delivery system. In some embodiments, the instructions require use of water-based lubricant, syringes, non-cutting, grasping forceps, and/or a flexible or rigid cystoscope. In some embodiments, the water-based lubricant, syringes, non-cutting, grasping forceps, and/or a flexible or rigid cystoscope are not included in the kit. In some embodiments, the kit comprises instructions for using the intravesical drug delivery system according to any of the methods provided herein.

In some embodiments, the kit provides instructions for inserting the intravesical drug delivery system, comprising: (1) Prepare for Placement: gather supplies, including Inserter, TAR-200, and two 10 mL slip tip syringes filled with water-based lubricant. If needed, fill syringes manually. Open TAR-200 outer foil pouch at tear notch. Open TAR-200 and Inserter Tyvek pouches from chevron side; (2) Introduce Empty Catheter into Bladder: (a) Lubricate tip of empty Catheter; (b) Introduce empty Catheter (without stylet) into urethra and advance until urine return. Note depth marking printed on Catheter to maintain position during procedure; (3) After Placing Catheter into Bladder, Inject Lubricant and Insert TAR-200: (a) Using first lubricant syringe, inject 2-3 mL of lubricant into Catheter's end; (b) Load either end of TAR-200 into Catheter and advance until completely uncoiled inside Catheter; (c) Using second lubricant syringe, inject another 2-3 mL of lubricant into Catheter's end; (d) Slowly insert stylet completely into Catheter until stylet hub is in flush contact with Catheter's end. This will ensure TAR-200 completely exits Catheter into bladder; I Remove Catheter and stylet from urethra together. TAR-200 should remain inside bladder. Note: If TAR-200 cannot be advanced due to resistance, remove catheter and stylet completely. Ensure TAR-200 is removed as well. Do not attempt to place the removed TAR-200 again.

In some embodiments, the kit provides instructions for inserting the intravesical drug delivery system, comprising: (1) Gather supplies, including the UPC and two sterile 10 ml slip tip syringes filled with sterile water-based lubricant. If needed, fill syringes manually; (2) Inspect all UPC device pouches for damage. DO NOT USE any product that is damaged or has damaged packaging; (3) After preparation of the urethral meatus, lubricate the tip of the empty catheter shaft. NOTE: Depth markings are aligned with upward direction of coude tip; (4) Introduce empty catheter shaft (without green stylet) into urethra and advance until urine return. Note depth marking printed on catheter shaft to maintain position during procedure; (5) Refer to the instructions for use provided with the intravesical drug delivery system on how to load the intravesical drug delivery system into the UPC; (6) Once the intravesical drug delivery system is loaded into the UPC, slowly insert the green stylet until the white stylet hub is in flush contact with the catheter's end. This will ensure that the intravesical drug delivery system completely exits the UPC into the bladder; (7) After withdrawing the catheter shaft, visually confirm that the intravesical drug delivery system is no longer in the UPC.

In some embodiments, the kit provides instructions for removing the intravesical drug delivery system, comprising: Insert Cystoscope and Grasp TAR-200: (a) Introduce cystoscope into bladder and visualize TAR-200, (b) Introduce grasping forceps through cystoscope's working channel, (c) Grasp TAR-200 over silicone tubing and wireform. Do not grasp on or near ends of TAR-200; and Remove TAR-200: (a) While grasping TAR-200, remove cystoscope and forceps out of urethra together to remove TAR-200 under direct vision. Do not remove TAR-200 through cystoscope's working channel. Doing so can damage TAR-200 and/or cystoscope; (b) After removal, inspect TAR-200 to confirm it is intact and unbroken.

In some aspects, provided herein is a kit comprising an intravesical drug delivery system provided herein. In some embodiments, the intravesical drug delivery system comprises an intravesical drug delivery system comprising 225-mg free base equivalents of gemcitabine. In some embodiments, the intravesical drug delivery system comprises a deployment shape and a retention shape. For example, the intravesical drug delivery system may be elastically deformable between a relatively straightened or uncoiled shape suited for insertion through a lumen (e.g., the urethra) into the bladder of the individual (the deployment shape) and a retention shape suited to retain the intravesical drug delivery system within the bladder. For the purposes of this disclosure, terms such as “relatively expanded shape,” “relatively higher-profile shape,” or “retention shape” generally denote any shape suited for retaining the intravesical drug delivery system in the intended implantation location, including but not limited to a pretzel or bi-oval shape or other coiled shape (e.g., comprising overlapping coils) that is suited for retaining the intravesical drug delivery system in the bladder. In some embodiments, the relatively expanded shape is a pretzel or bi-oval shape. In some embodiments, the kit further comprises a Urinary Placement Catheter.

In some embodiments, the kit further comprises instructions for administering the intravesical drug delivery system according to any of the methods disclosed herein.

In some embodiments, the kit comprises instructions for a method of treating individuals with high-risk non-muscle invasive bladder cancer comprising administering gemcitabine locally to the bladder of the individual, wherein each gemcitabine administration comprises delivering gemcitabine continuously to the bladder for at least 7 days during a gemcitabine delivery period. In some embodiments, gemcitabine is administered to the bladder about every three weeks for about the first six months and about every three months thereafter. In some embodiments, gemcitabine is administered to the bladder about every three weeks for about the first 24 weeks and then about every three months thereafter for a total of about two years. In some embodiments, about 225 mg of gemcitabine is administered during each administration. In some embodiments, the individual has BCG unresponsive non-muscle invasive bladder cancer. In some embodiments, the individual has refused or is ineligible for radical cystectomy.

In some embodiments, provided herein is a kit comprising Cetrelimab 240 mg Final Lyophilized Product (e.g., provided as 240 mg/vial, which can be reconstituted in 30 mL of solution). In some embodiments, the kit further comprises instructions for reconstitution of the lyophilized product. In some embodiments, the kit further provides instructions for dilution of the reconstituted product with 0.9% sodium chloride injection to make 100 mL IV solution and to administer the cetrelimab via IV infusion.

In some aspects, provided herein is a kit comprising a PD-1 antibody described herein. In some embodiments, the anti-PD-1 antibody is cetrelimab. In some embodiments, the cetrelimab is provided in a pharmaceutical composition suitable for intravenous administration, such as any one of the pharmaceutical compositions described herein.

In some embodiments, the kit further comprises instructions for administering an anti-PD-1 antibody or antigen-binding fragment thereof according to any of the methods disclosed herein.

In some aspects, provided herein is kit comprising an antibody that binds to PD-1 and a package insert comprising instructions for use according to the methods disclosed herein. In some embodiments, the PD-1 antibody is packaged with an infusion pump and an intravenous administration set (IV set). In some embodiments, the antibody is cetrelimab. In some embodiments, the antibody is lyophilized. In some embodiments, the antibody Is packaged as a liquid formulation in a vial. In some embodiments, the cetrelimab is provided in a pharmaceutical composition suitable for intravenous administration, such as any one of the pharmaceutical compositions described herein. In some embodiments, the instructions for use comprise instructions for administering an antibody that binds to PD-1.

In some embodiments, the kit comprises instructions for a method of treating individuals with high-risk non-muscle invasive bladder cancer comprising administering to the individual an antibody that binds to PD-1 about every three weeks for the duration of the dosing period. In some embodiments, the antibody that binds to PD-1 is administered about every three weeks for about the first 18 months. In some embodiments, about 360 mg of the PD-1 antibody is administered (e.g., via intravenous infusion) during each administration. In some embodiments, the individual has BCG unresponsive non-muscle invasive bladder cancer. In some embodiments, the individual has refused or is ineligible for radical cystectomy.

### EMBODIMENTS

Various embodiments of the methods, systems, articles of manufacture, and kits for treating muscle invasive bladder cancer in an individual provided herein are included in the following non-limiting list of embodiments.
1. A method of treating high-risk non-muscle invasive bladder cancer (HR-NMIBC) that is unresponsive to intravesical Bacillus Calmette-Guerin (BCG) therapy in an individual comprising administering to the bladder of the individual about 225 mg of gemcitabine about every three weeks (Q3W) for at least about 24 weeks, wherein such treatment results in a complete response rate in a population of patients who have received such treatment of at least 50%.
2. A method of bladder sparing in an individual having high-risk non-muscle invasive bladder cancer (HR-NMIBC) that is unresponsive to intravesical Bacillus Calmette-Guerin (BCG) therapy comprising administering to the bladder of the individual about 225 mg of gemcitabine about every three weeks (Q3W) for at least about 24 weeks, wherein the individual does not receive a radical cystectomy.
3. A method of treating high-risk non-muscle invasive bladder cancer (HR-NMIBC) that is unresponsive to intravesical Bacillus Calmette-Guerin (BCG) therapy in an individual who is eligible for radical cystectomy, the method comprising administering to the bladder of the individual about 225 mg of gemcitabine about every three weeks (Q3W) for at least about 24 weeks.
4. A method of treating high-risk non-muscle invasive bladder cancer (HR-NMIBC) that is unresponsive to intravesical Bacillus Calmette-Guerin (BCG) therapy in an individual comprising inserting an intravesical drug delivery system comprising about 225 mg gemcitabine into the bladder of the individual and removing the intravesical drug delivery system about three weeks later for at least about 24 weeks, wherein such treatment results in a complete response rate in a population of patients who have received such treatment of at least 50%.
5. A method of increasing the complete response rate for treating high-risk non-muscle invasive bladder cancer (HR-NMIBC) that is unresponsive to intravesical Bacillus Calmette-Guerin (BCG) therapy in an individual comprising administering to the individual about 225 mg of gemcitabine about every three weeks (Q3W) for at least about 24 weeks, wherein such treatment results in a complete response rate that is at least 1.5 fold of the complete response rate achieved by valrubicin in a population of patients who have received such treatment.
6. A method of inducing a complete response (CR) rate of about 72.7% in a patient population with high-risk non-muscle invasive bladder cancer (HR-NMIBC) that is unresponsive to intravesical Bacillus Calmette-Guerin (BCG) therapy, the method comprising inserting an intravesical drug delivery system comprising about 225 mg gemcitabine into the bladder of an individual with HR-NMIBC, and removing the intravesical drug delivery system about three weeks later for at least about 24 weeks.
7. A method of inducing a complete response (CR) rate of about 76.7% in a patient population with high-risk non-muscle invasive bladder cancer (HR-NMIBC) that is unresponsive to intravesical Bacillus Calmette-Guerin (BCG) therapy, the method comprising inserting an intravesical drug delivery system comprising about 225 mg gemcitabine into the bladder of an individual with HR-NMIBC, and removing the intravesical drug delivery system about three weeks later for at least about 24 weeks.
8. A method of treating high-risk non-muscle invasive bladder cancer (HR-NMIBC) that is unresponsive to intravesical Bacillus Calmette-Guerin (BCG) therapy in an individual comprising administering to the bladder of the individual about 225 mg of gemcitabine about every three weeks (Q3W) for at least about 24 weeks, wherein such treatment results in an increased complete response rate compared to the complete response rate achieved by valrubicin.
9. A method of treating high-risk non-muscle invasive bladder cancer (HR-NMIBC) that is unresponsive to intravesical Bacillus Calmette-Guerin (BCG) therapy in an individual comprising administering gemcitabine to the bladder of the individual every three weeks (Q3W) for at least about 24 weeks during eight administration periods, wherein each administration period comprises a delivery period of at least seven days when gemcitabine is released from an intravesical drug delivery system, wherein the concentration of gemcitabine in the urine of the individual is at least 4 μg/mL during each delivery period.
10. The method of embodiment 9, wherein the concentration of gemcitabine in the urine of the individual is between 4 μg/mL and 50 μg/mL during each administration period.
11. A method of treating high-risk non-muscle invasive bladder cancer (HR-NMIBC) that is unresponsive to intravesical Bacillus Calmette-Guerin (BCG) therapy comprising (a) administering to the bladder of the individual about 225 mg of gemcitabine during an administration period about every three weeks (Q3W) for at least about 24 weeks, (b) inducing a complete response, and wherein each administration period comprises a delivery period of at least seven days.
12. The method of any one of embodiments 1-3, 5, and 8-11, wherein about 225 mg of gemcitabine is administered to the individual during an administration period.
13. The method of embodiment 12, wherein each administration period is about three weeks.
14. The method of embodiment 12 or 13, wherein each administration period begins with inserting a drug delivery system comprising about 225 mg gemcitabine into the bladder and each administration period ends with removing the drug delivery system from the bladder.
15. The method of any one of embodiments 1-3, 5, and 8-14, wherein gemcitabine is released into the bladder during a delivery period.
16. The method of embodiment 14, wherein the delivery period is less than the administration period.
17. The method of embodiment 14 or embodiment 15, wherein the delivery period is one week to three weeks.
18. The method of any one of embodiments 1-17, wherein the gemcitabine administered to the individual is a monotherapy for treatment of the HR-NMIBC that is unresponsive to intravesical BCG therapy.
19. The method of any one of embodiments 2, 3, 5, or 8-10, wherein such treatment results in a complete response rate in a population of patients who have received such treatment of at least 50%.
20. The method of any one of embodiments 1, 4, or 12, wherein the complete response rate in the population of patients is about 60% to about 87%.
21. The method of any one of embodiments 1, 4, or 12, wherein the complete response rate in the population of patients is between about 71% to about 91%.
22. The method of any one of embodiments 1, 4, 12, 20, or 21, wherein the complete response rate in the population of patients is about 87%, about 83%, about 82%, about 79%, about 77%, or about 73%.
23. The method of any one of embodiments 1˜4 or 6-22, wherein the complete response rate in the population of patients is improved compared to the standard of care, optionally wherein the complete response rate in the population of patients is improved at least 1.5 fold compared to the complete response rate for standard of care.
24. The method of any one of embodiments 1˜4 or 6-23, wherein the complete response rate in the population of patients is improved compared to a checkpoint inhibitor therapy, optionally wherein the complete response rate is improved at least 1.5 fold compared to the complete response rate for the checkpoint inhibitor therapy.
25. The method of any one of embodiments 1˜4 or 6-24, wherein the complete response rate in the population of patients is improved compared to treatment with valrubicin, optionally wherein the complete response rate is improved at least 1.5 fold compared to the complete response rate for valrubicin.
26. The method of any one of embodiments 1-25, wherein the individual has a complete response within three months of the start of treatment with gemcitabine.
27. A method of treating high-risk non-muscle invasive bladder cancer (HR-NMIBC) that is unresponsive to intravesical Bacillus Calmette-Guerin (BCG) therapy in an individual comprising administering to the individual about 360 mg of an antibody that binds to PD-1 about every three weeks (Q3W) for about 6 months, wherein the antibody that binds to PD-1 comprises a variable heavy chain (VH) and a variable light chain (VL), wherein the VH comprises a CDRH1 comprising the amino acid sequence set forth in SEQ ID NO:1, a CDRH2 comprising the amino acid sequence set forth in SEQ ID NO:2, a CDRH3 comprising the amino acid sequence set forth in SEQ ID NO:3; and the VL comprises a CDRL1 comprising the amino acid sequence set forth in SEQ ID NO: 4, a CDRL2 comprising the amino acid sequence set forth in SEQ ID NO: 5, and a CDRL3 comprising the amino acid sequence set forth in SEQ ID NO: 6.
28. The method of embodiment 26, wherein the complete response rate is at least 30%.
29. The method of embodiment 27 or 28, wherein the complete response rate is about 33%.
30. The method of any one of embodiments 27-29, wherein the antibody that binds to PD-1 is administered to the individual as a monotherapy for treatment of the high-risk non-muscle invasive bladder cancer (HR-NMIBC) that is unresponsive to intravesical Bacillus Calmette-Guerin (BCG) therapy.
31. The method of any one of embodiments 27-30, wherein the antibody that binds to PD-1 is administered every three weeks (Q3W) for about 18 months.
32. The method of any one of embodiments 1, 4-6, 11-31, wherein a complete response is determined on the basis of a negative cytology analysis.
33. The method of any one of embodiments 1, 4-6, 11-31, wherein a complete response is determined on the basis of a negative biopsy.
34. The method of any one of embodiments 1-25, or 32-33, wherein the individual is administered gemcitabine within 12 months of completion of the last dose of BCG therapy.
35. The method of any one of embodiments 1-25, or 32-34, wherein the individual has received i) a minimum of five of six full doses of an induction course of BCG; and ii) two or three doses of a maintenance course, or two of six doses of a second induction course of BCG therapy.
36. The method of any one of embodiments 1-35, wherein the individual has carcinoma in situ (CIS) and/or wherein the population of patients comprises one or more individuals with CIS.
37. The method of any one of embodiments 1-36, wherein the individual has a histologically confirmed diagnosis of persistent or recurrent CIS and/or wherein the population of patients comprises one or more individuals with a histologically confirmed diagnosis of persistent or recurrent CIS.
38. The method of any one of embodiments 1-37, wherein the individual has Ta stage bladder cancer, and/or wherein the population of patients comprises one or more individuals with Ta stage bladder cancer.
39. The method of embodiment 38, wherein the individual has high grade Ta stage bladder cancer and/or wherein the population of patients comprises one or more individuals with high grade Ta stage bladder cancer.
40. The method of any one of embodiments 1-37, wherein the individual has T1 stage bladder cancer and/or wherein the population of patients comprises one or more individuals with T1 stage bladder cancer.
41. The method of embodiment 40, wherein the individual has high grade T1 stage bladder cancer and/or wherein the population of patients comprises one or more individuals with high grade T1 stage bladder cancer.
42. The method of any one of embodiments 1-41, wherein the individual has papillary bladder cancer and/or wherein the population of patients comprises one or more individuals with papillary bladder cancer.
43. The method of any one of embodiments 1-42, wherein the individual has a mixed histology tumor and/or wherein the population of patients comprises one or more individuals with a mixed histology tumor.
44. The method of any one of embodiments 1-43, wherein the individual is ineligible or elected not to undergo radical cystectomy.
45. The method of embodiment 44, wherein the individual elected not to undergo radical cystectomy due to bladder preservation or concern about quality of life after bladder removal.
46. The method of any one of embodiments 1-45, wherein the bladder cancer does not have neuroendocrine, micropapillary, signet ring cell, plasmacytoid, or sarcomatoid features.
47. The method of any one of embodiments 1-46, wherein the individual does not have a urothelial carcinoma or histological variant at any site outside of the urinary bladder.
48. The method of any one of embodiments 1-47, comprising performing a transurethral resection of bladder tumor (TURBT) if the individual has papillary bladder cancer.
49. The method of embodiment 48, wherein the individual has carcinoma in situ (CIS) following TURBT.
50. The method of any one of embodiments 1-49, wherein following treatment with gemcitabine, the individual is monitored for recurrence.
51. The method of any one of embodiments 1-26 or 32-50, wherein following treatment with gemcitabine, the individual does not receive valrubicin, systemic gemcitabine, docetaxel, pembrolizumab or paclitaxel for treating the high-risk non-muscle invasive bladder cancer (HR-NMIBC) that is unresponsive to intravesical Bacillus Calmette-Guerin (BCG) therapy.
52. The method of any one of embodiments 1-26 or 32-51, wherein treatment with gemcitabine causes tumor ablation.
53. The method of any one of embodiments 1-26 or 32-52, wherein following treatment with gemcitabine, the individual does not receive a transurethral resection of bladder tumor (TURBT).
54. The method of any one of embodiments 1-26 or 32-53, wherein the individual does not progress to muscle-invasive bladder cancer.
55. The method of any one of embodiments 1-26 or 32-54, wherein the quality-of-life score of the individual is maintained or increases following treatment with gemcitabine.
56. The method of any one of embodiments 1-26 or 32-55, wherein the individual has asymptomatic macrohematuria or dysuria prior to the treatment with gemcitabine.
57. The method of any one of embodiments 1-3, 5, 8-26 or 32-56, wherein administering gemcitabine comprises inserting an intravesical drug delivery system comprising about 225 mg gemcitabine into the bladder of the individual and removing the intravesical drug delivery system about three weeks later.
58. The method of any one of embodiments 1-26 or 32-56, comprising i) inserting an intravesical drug delivery system comprising about 225 mg of gemcitabine into the bladder of the individual; ii) removing the intravesical drug delivery system about three weeks later; iii) inserting a new intravesical drug delivery system comprising about 225 mg of gemcitabine into the bladder on the day that the intravesical drug delivery system is removed in step ii), iv) removing the intravesical drug delivery system about three weeks later; wherein steps ii) and iii) are completed seven times.
59. The method of any one of embodiments 1-26 or 32-56, comprising a dosing schedule of at least 24 weeks comprising i) inserting a first intravesical drug delivery system into the bladder in week 0, and removing the first intravesical drug delivery system in week 3; ii) inserting a second intravesical drug delivery system into the bladder in week 3 after removing the first intravesical drug delivery system, and removing the second intravesical drug delivery system in week 6; iii) inserting a third intravesical drug delivery system into the bladder in week 6 after removing the second intravesical drug delivery system, and removing the third intravesical drug delivery system in week 9; iv) inserting a fourth intravesical drug delivery system into the bladder in week 9 after removing the third intravesical drug delivery system, and removing the fourth intravesical drug delivery system in week 12; v) inserting a fifth intravesical drug delivery system into the bladder in week 12 after removing the fourth intravesical drug delivery system, and removing the fifth intravesical drug delivery system in week 15; vi) inserting a sixth intravesical drug delivery system into the bladder in week 15 after removing the fifth intravesical drug delivery system, and removing the sixth intravesical drug delivery system in week 18; vii) inserting a seventh intravesical drug delivery system into the bladder in week 18 after removing the sixth intravesical drug delivery system, and removing the seventh intravesical drug delivery system in week 21; viii) inserting an eighth intravesical drug delivery system into the bladder in week 21 after removing the seventh intravesical drug delivery system, and removing the eighth intravesical drug delivery system in week 24; wherein each intravesical drug delivery system comprises an intravesical device comprising about 225 mg of gemcitabine.
60. The method of any one of embodiments 1-26 or 32-59, further comprising a maintenance therapy phase, wherein the maintenance therapy phase comprises administering about 225 mg of gemcitabine to the bladder of the individual for about three weeks about every two to four months.
61. The method of embodiment 60, wherein the maintenance therapy phase comprises administering about 225 mg gemcitabine to the bladder of the individual for about three weeks about every three months.
62. The method of embodiment 60 or embodiment 61, wherein the maintenance therapy phase begins at least 30 weeks after the start of treatment with gemcitabine.
63. The method of embodiment 62, wherein the maintenance therapy begins about 36 weeks after the start of treatment with gemcitabine.
64. The method of any one of embodiments 60-63, wherein the maintenance therapy comprises administering about 225 mg gemcitabine to the bladder of the individual during weeks 36-39, 48-51, 60-63, 72-75, 84-87, or 96-99.
65. The method of any one of embodiments 60-64, comprising i) inserting an intravesical drug delivery system into the bladder of the individual on about week 36 and removing the intravesical drug delivery system on about week 39; ii) inserting an intravesical drug delivery system into the bladder of the individual on about week 48 and removing the intravesical drug delivery system on about week 51; iii) inserting an intravesical drug delivery system into the bladder of the individual on about week 60 and removing the intravesical drug delivery system on about week 63; iv) inserting an intravesical drug delivery system into the bladder of the individual on about week 72 and removing the intravesical drug delivery system on about week 75; v) inserting an intravesical drug delivery system into the bladder of the individual on about week 84 and removing the intravesical drug delivery system on about week 87; vi) inserting an intravesical drug delivery system into the bladder of the individual on about week 96 and removing the intravesical drug delivery system on about week 99; wherein the intravesical drug delivery system comprises an intravesical device comprising about 225 mg of gemcitabine.
66. The method of any one of embodiments 57-65, wherein gemcitabine is delivered from the intravesical device during a delivery period.
67. The method of embodiment 66, wherein the delivery period is one to three weeks.
68. The method of embodiment 67, wherein the concentration of gemcitabine in the urine is from about 1 μg/mL to about 25 μg/mL or from about 4 μg/mL to about 50 μg/mL during the delivery period.
69. The method of any one of embodiments 4, 6, 9, or 57-68, wherein the intravesical drug delivery system releases gemcitabine at a rate of 10 mg/day to 45 mg/day for the first seven days of administration.
70. The method of any one of embodiments 4, 6, 9, or 57-69, wherein the intravesical drug delivery system comprises an intravesical device that comprises a housing which contains and controllably releases the gemcitabine and is elastically deformable between a retention shape configured to retain the intravesical device in the individual's bladder and a deployment shape for passage of the intravesical device through the individual's urethra.
71. The method of embodiment 70, wherein the intravesical device comprises a dual lumen silicon part with a single laser-machined orifice.
72. The method of embodiment 71, wherein the dual lumen silicon part comprises a large lumen and a small lumen.
73. The method of embodiment 72, wherein the small lumen comprises a super-elastic nitinol wire.
74. The method of embodiment 73, wherein the super-elastic nitinol wire is in a predefined form to provide retention of the system in the bladder.
75. The method of any one of embodiments 72-74, wherein the large lumen of the dual lumen silicon part comprises the gemcitabine.
76. The method of any one of embodiments 72-75, wherein the large lumen of the dual lumen silicon part comprises osmotic minitablets.
77. The method of embodiment 76, wherein the osmotic minitablets contain urea as an osmotic agent.
78. The method of any one of embodiments 4, 6, 9, or 57-77, wherein the intravesical drug delivery system is deployed into the bladder of the individual by a urinary placement catheter.
79. The method of any one of embodiments 1-78, wherein the median duration of response in the population of patients is not reached within about 10.6 months.
80. The method of any one of embodiments 1-78, wherein the median duration of response in the population of patients is not reached within about 48 weeks.
81. The method of any one of embodiments 1-79, wherein such treatment results in a 6-month event-free survival in the population of patients of at least 85%.
82. The method of any one of embodiments 1-79, wherein the 6-month event-free survival in the population of patients is between about 69% to about 95%.
83. The method of embodiment 81 or embodiment 82, wherein such treatment results in a 6-month event-free survival in the population of patients of about 87% or about 93%.
84. The method of any one of embodiments 1-83, wherein such treatment results in a 12-month event-free survival in the population of patients of at least 70%.
85. The method of any one of embodiments 1-83, wherein the 12-month event-free survival in the population of patients is between about 50% to about 88%.
86. The method of embodiment 84 or embodiment 85, wherein such treatment results in a 12-month event-free survival in the population of patients of about 75% or about 84%.
87. The method of any one of embodiments 1-86, wherein such treatment results in an 18-month event-free survival in the population of patients of at least 70%.
88. The method of any one of embodiments 1-86, wherein such treatment results in an 18-month event-free survival in the population of patients between about 50% to about 88%.
89. The method of embodiment 87 or embodiment 88, wherein such treatment results in an 18-month event-free survival in the population of patients of about 75% or about 84%.
90. The method of any one of embodiments 1-89, wherein such treatment results in a 24-month event-free survival in the population of patients of at least 70%.
91. The method of any one of embodiments 1-89, wherein such treatment results in a 24-month event-free survival in the population of patients between about 50% to about 88%.
92. The method of embodiment 90 or embodiment 91, wherein such treatment results in a 24-month event-free survival in the population of patients of about 75%.
93. A kit comprising an intravesical drug delivery system comprising about 225 mg of gemcitabine and instructions for use comprising the method of any one of embodiments 1-26 or 32-92.
94. The kit of embodiment 93, further comprising a urinary placement catheter.
95. The kit of embodiment 94, wherein the urinary placement catheter comprises a catheter and a stylet.
96. The kit of any one of embodiments 93-95, further comprising a lubricant and/or a syringe.
97. An article of manufacture comprising an intravesical drug delivery system comprising an intravesical device comprising about 225 mg of gemcitabine and a package insert comprising instructions for use according to the method of any one of embodiments 1-26 or 32-92.
98. An intravesical drug delivery system comprising an intravesical device comprising about 225 mg gemcitabine for use in the method of any one of embodiments 1-26 or 32-92.
99. Gemcitabine for use according to the method of any one of embodiments 1-26 or 32-92.
100. Use of gemcitabine for treating high-risk non-muscle invasive bladder cancer (HR-NMIBC) that is unresponsive to intravesical Bacillus Calmette-Guerin (BCG) therapy in an individual comprising administering to the bladder of the individual about 225 mg of gemcitabine about every three weeks (Q3W) for at least about 24 weeks, wherein such treatment results in a complete response rate in a population of patients who have received such treatment of at least 50%.
101. The method of any one of embodiments 1-26 or 32-92, wherein the gemcitabine is gemcitabine free base equivalent (FBE).
102. The method of any one of embodiments 1-26, 32-92, or 101, wherein a) less than about 91% of individuals within the population of patients experience an adverse effect; b) less than about 35% of individuals within the population of patients experience a greater than grade 3 adverse effect; c) less than about 40% of individuals within the population of patients experience micturition urgency; d) less than about 40% of individuals within the population of patients experience pollakiuria; e) less than about 30% of individuals within the population of patients experience noninfective cystitis; f) less than about 30% of individuals within the population of patients experience a urinary tract infection; g) less than about 5% of individuals within the population of patients experience pruritus; h) less than about 5% of individuals within the population of patients experience diarrhea; i) less than about 30% of individuals within the population of patients experience dysuria; j) less than about 15% of individuals within the population of patients experience hematuria; k) less than about 10% of individuals within the population of patients experience urinary tract pain; 1) less than about 10% of individuals within the population of patients experience urinary retention; m) less than about 5% of individuals within the population of patients experience renal impairment; and/or n) less than about 5% of individuals within the population of patients experience urosepsis.
103. Minitablets comprising gemcitabine for use according to the method of any one of embodiments 1-26, 32-92, or 101-102.
104. Gemcitabine for use with an intravesical device, for use according to the method of any one of embodiments 1-26, 32-92, or 101-102.
105. Gemcitabine for use according to the method of any one of embodiments 1-26, 32-92, 101-102, in combination with an intravesical device.
106. Gemcitabine for use according to the method of any one of embodiments 1-26, 32-92, 101-102, wherein the gemcitabine is administered by an intravesical device.
107. The method of any one of embodiments 27-49, wherein the VH comprises the amino acid sequence set forth in SEQ ID NO: 7 and the VL comprises the amino acid sequence set forth in SEQ ID NO:8.
108. The method of any one of embodiments 27-49 or 107, wherein antibody that binds to PD-1 comprises a heavy chain comprising the amino acid sequence set forth in SEQ ID NO:9 and the light chain comprising the amino acid sequence set forth in SEQ ID NO: 10.
109. The method of any one of embodiments 27-49, 107, or 108, wherein the antibody that binds to PD-1 is cetrelimab.
110. The method of any one of embodiments 27-49, or 107-109, wherein the antibody that binds to PD-1 is administered intravenously.
111. The method of any one of embodiments 1-26, 32-92, 101-102, wherein amount of gemcitabine administered to the individual is a clinically effective amount of gemcitabine.
112. The method of any one of embodiments 1-26, 32-92, 101-102, or 111, wherein there are at least 20 individuals in the population of patients.
113. The method of any one of embodiments 1-26, 32-92, 101-102, or 111-112, wherein there are 23 individuals in the population of patients.
114. The method of any one of embodiments 1-26, 32-92, 101-102, or 111-112, wherein there are 54 individuals in the population of patients.
115. The method of any one of embodiments 1-26, 32-92, 101-102, or 111-112, wherein there are 85 individuals in the population of patients.
116. The method of any one of embodiments 1-26, 32-92, 101-102, or 111-115, wherein the individual is ineligible for radical cystectomy due to age, high American Society of Anesthesiologists class score, or medical and surgical comorbidities.
117. The method of any one of embodiments 1-26, 32-92, 101-102, or 111-116, wherein the individual elects not to undergo a radical cystectomy to preserve their bladder, to preserve sexual function, concern about quality of life after cystectomy, or concern about mortality and morbidity risk associated with radical cystectomy.
118. The method of any one of embodiments 1-26, 32-92, 101-102, or 111-117, wherein about 51% of patients in the population of patients refuse radical cystectomy to preserve their blader.
119. The method of any one of embodiments 1-26, 32-92, 101-102, or 111-118, wherein about 37% of patients in the population of patients refuse radical cystectomy because of morbidity and mortality concerns.
120. The method of any one of embodiments 4, 6, 7, 9, 10, 12-26, 32-92, 101-102, or 111-119, wherein the intravesical drug delivery system is a TAR-200 intravesical drug delivery system.
121. The method of any one of embodiments 1-26, 32-92, 101-102, or 111-120, wherein the median duration of response in the population of patients is about 26 months.
122. The method of any one of embodiments 1-26, 32-92, 101-102, or 111-121, wherein such treatment results in a one-year duration of response (DOR) rate in the population of patients of at least 50%.
123. The method of embodiment 122, wherein one-year duration of response (DOR) rate in the population of patients is between about 50% to about 89%.
124. The method of embodiment 122 or embodiment 123, wherein the one-year DOR rate is about 77%.
125. The method of any one of embodiments 1-26, 32-92, 101-102, or 111-124, wherein the individual is at risk of disease progression.
126. The method of any one of embodiments 1-26, 32-92, 101-102, or 111-125, wherein the individual has an ECOG performance status of 0-2.
127. The method of any one of embodiments 1-26, 32-92, 101-102, or 111-126, wherein such treatment results in a 12-month CR rate in the population of patients of at least 40%.
128. The method of embodiment 127, wherein the 12-month CR rate in the population of patients is between about 43% to about 79%.
129. The method of embodiment 127 or embodiment 128, wherein the 12-month CR rate in the population of patients is about 64%.
130. The method of any one of embodiments 1-26, 32-92, 101-102, or 111-129, wherein such treatment results in a 15-month CR rate in the population of patients of at least 40%.
131. The method of embodiment 130, wherein the 15-month CR rate in the population of patients is between about 43% to about 79%.
132. The method of embodiment 130 or embodiment 131, wherein the 15-month CR rate in the population of patients is about 64%.

### EXAMPLES

The following examples are included for illustrative purposes only and are not intended to limit the scope of the present disclosure.

### Example 1

### Study Design

This study was an open-label, parallel-group, multi-center study evaluating the efficacy and safety of intravesical TAR-200 alone, systemic intravenous (IV) cetrelimab alone, or intravesical TAR-200 in combination with systemic IV cetrelimab in participants with HR NMIBC (CIS with or without papillary disease) unresponsive to prior intravesical BCG therapy, who are either ineligible for or have elected not to undergo RC after making an informed decision. Participants were randomly assigned (1:1:2) to receive intravesical-TAR-200 alone (Cohort 1), IV cetrelimab alone (Cohort 2), or intravesical TAR-200 in combination with systemic IV cetrelimab (Cohort 3). The amounts provided were hoped to be clinically effective amounts, however analysis of the data generated during this study would be required to conclude that the endpoints were met.

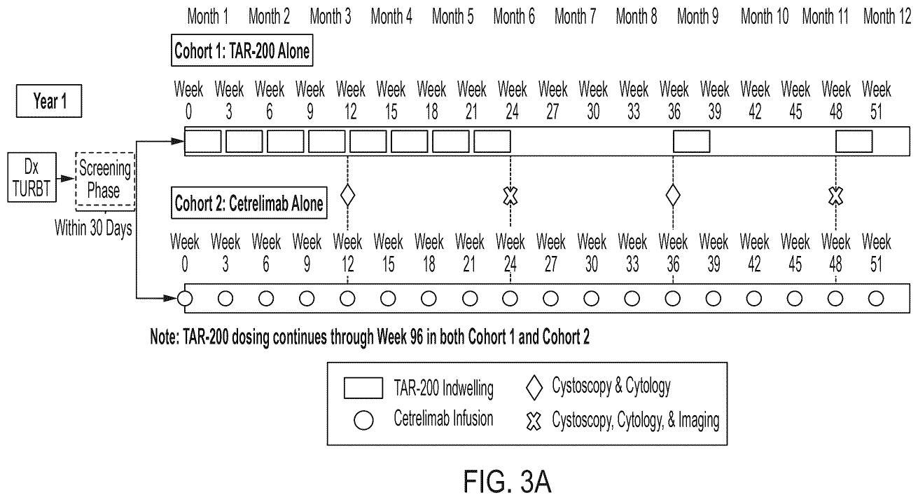

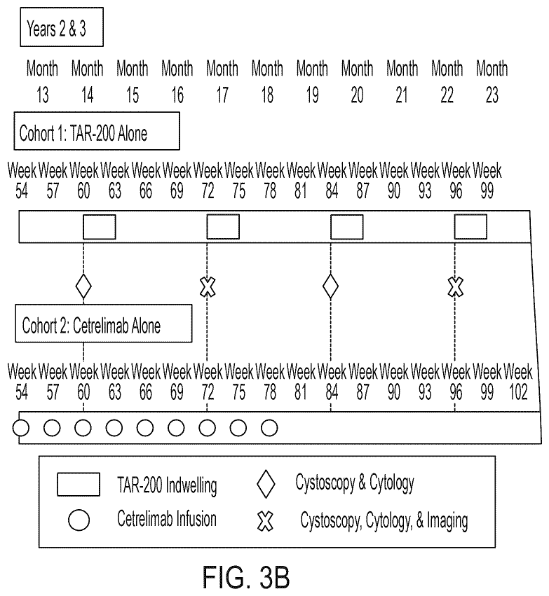

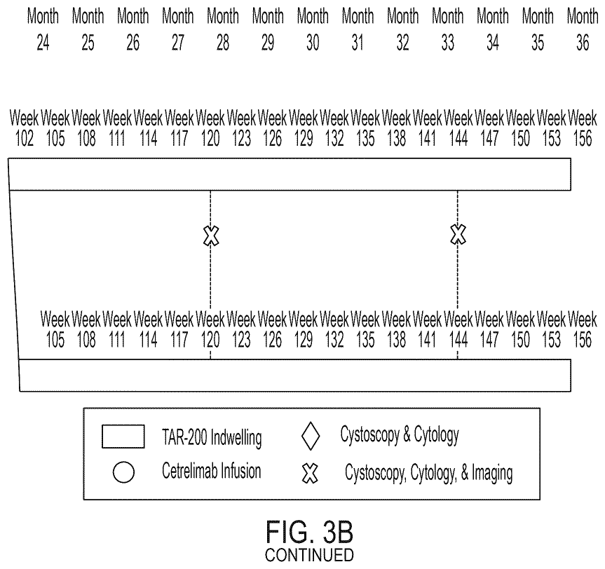

For Cohort 1, TAR-200 alone was dosed every 3 weeks (Q3W) for up to the first 24 weeks (6 months) on study, as shown in FIG. 3A. Dosing of TAR-200 then occurred every 12 weeks through Week 99 (Year 2, as shown in FIG. 3B). Each TAR-200 was removed after a 3-week dwell time through Week 99 (Year 2). For Cohort 2, cetrelimab was dosed at 360 mg IV Q3W through Week 78 (18 months), as shown in FIGS. 3A and 3B. For Cohort 3, TAR-200 was dosed Q3W for up to the first 24 weeks (6 months), as shown in FIG. 3A. Dosing of TAR-200 then occurred every 12 weeks through Week 99 (Year 2, as shown in FIG. 3B). Each TAR-200 was removed after a 3-week dwell time through Week 99 (Year 2). Cetrelimab was dosed at 360 mg IV Q3W through Week 78 (18 months). Dosing of TAR-200 then occurred every 12 weeks through Week 99 (Year 2).

The following analysis was performed on data from patients in Cohort 1 and Cohort 2 treated through 20 Feb. 2023 (clinical cut-off date).

### Study Primary Objective

The primary study objective was to evaluate the overall complete response (CR) rate in participants treated with TAR-200 alone (Cohort 1), or IV cetrelimab alone (Cohort 2) with Carcinoma in Situ (CIS), with or without concomitant high-grade Ta or T1 papillary disease.

### Study Primary Endpoint

The primary endpoint evaluated in this study was the complete response (CR) rate. The CR rate was defined as the proportion of participants who met at least one of the following: locally assessed negative cystoscopy and negative (including atypical) centrally read urine cytology, or positive cystoscopy with biopsy-proven benign or low-grade NMIBC and negative (including atypical) centrally read urine cytology at any time point.

### Study Secondary Objectives

The secondary objectives of this study included the following. 1) To evaluate the duration of response (DOR) in participants treated with TAR-200 alone (Cohort 1), or IV cetrelimab alone (Cohort 2) with CIS (with or without concomitant Ta or T1 papillary disease) who achieve a CR. 2) To determine the overall survival (OS) in all participants, defined as the time from the date of first dose of study treatment to death; if a participant has not died at the time of analysis, the participant will be censored at the date last known alive. 3) To evaluate the pharmacokinetics (PK) of gemcitabine and major metabolite 2′,2′ difluorodeoxyuridine (dFdU) in urine and plasma in TAR-200 alone (Cohort 1). 4) To evaluate the PK and immunogenicity of IV cetrelimab in serum in cetrelimab alone (Cohort 2). 5) To evaluate health-related quality of life (HRQoL) in all participants. 6) To assess the safety and tolerability of participants receiving TAR-200 alone (Cohort 1), or IV cetrelimab alone (Cohort 2).

### Overview

Safety analyses were performed on the treated analysis set defined as all randomized participants who received at least one dose of any study intervention. Summaries of treatment-emergent adverse events (TEAEs), serious TEAEs, TEAEs by toxicity grade, study drug/device/procedure related TEAEs, and TEAEs leading to treatment discontinuation or death, if any, are provided.

The efficacy analysis presented is limited to the primary efficacy measure in this study, CR rate, and preliminary data on the duration of response. It was performed on the evaluable analysis set defined as all treated participants (receiving at least one dose of any study drug) who have active disease at baseline and adequate disease assessment (centrally assessed urine cytology, tumor biopsy (when available) and cystoscopy) from at least one post-baseline disease assessment visit (from approximately 12 weeks since start of treatment), or who have progressed, died, or been withdrawn from treatment due to the recurrence of high-risk disease or progressive disease. The summary of overall complete response (CR) rate for each cohort is provided.

### Subject and Treatment Information

Participants were randomized to 1 of 3 cohorts in a 1:1:2 ratio and received treatment: 22 in Cohort 1 (TAR-200 alone), 23 in Cohort 2 (cetrelimab alone), and 43 in cohort 3 (TAR-200+cetrelimab) with median follow-up times of 40.9 weeks for cohort 1, 39.9 weeks for cohort 2, and 40.1 weeks for cohort 3.

The primary reasons for refusal included preservation of bladder and concern about quality of life after cystectomy. Additionally, 2 participants were ineligible for radical cystectomy (RC) with the primary reasons being advanced age (1 participant) and underlying medical and surgical comorbidities (1 participant). Three subjects were missing a reason for not receiving RC. At baseline, participants had carcinoma in situ (CIS) only, had CIS with concurrent Ta disease, or had CIS with concurrent T1 histology.

In the treated analysis set, at the clinical cut-off, the median (range) number of treatment cycles was 7.5 (1; 13) in cohort 1, and 5.0 (2; 25) in cohort 2. In cohort 1, the median (range) number of TAR-200 administrations received was 7.5 (1; 13) with median treatment duration of 23.7 weeks, and in cohort 2 the median (range) number of cetrelimab administrations received was 5.0 (2; 25) with median treatment duration of 12.3 weeks.

Twenty-one of the 43 (48.8%) participants in cohort 3 are ongoing at the clinical cutoff date with at least one of treatment (TAR-200 or/and cetrelimab). In cohort 1, 15 (68.2%) participants are ongoing with TAR-200, and in cohort 2, 9 (39.1%) participants are ongoing with cetrelimab.

### Efficacy

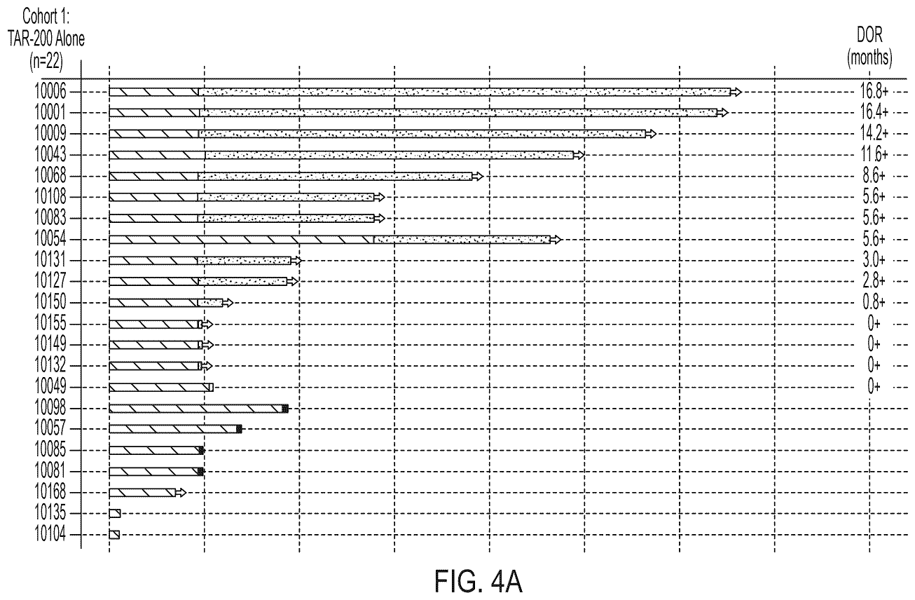

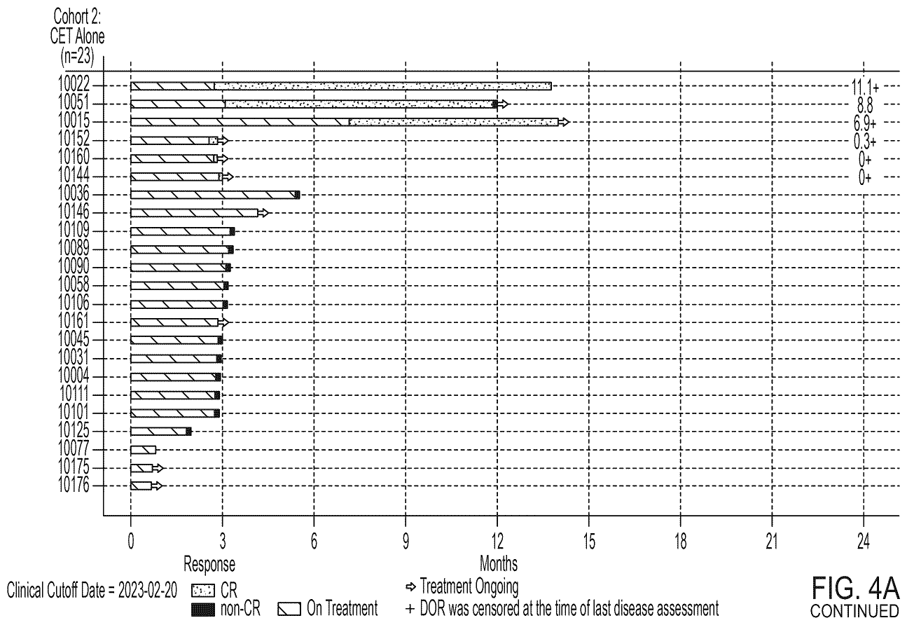

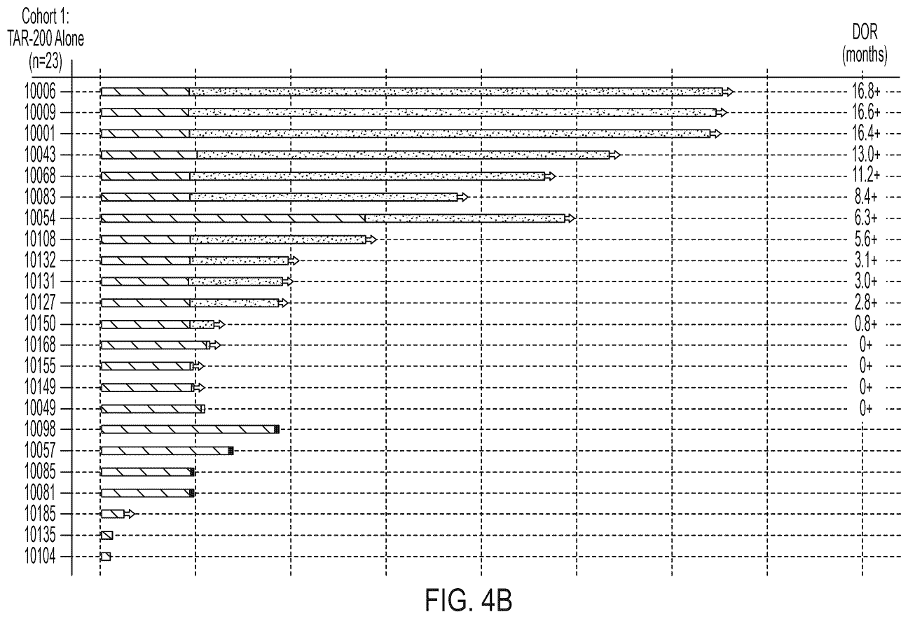

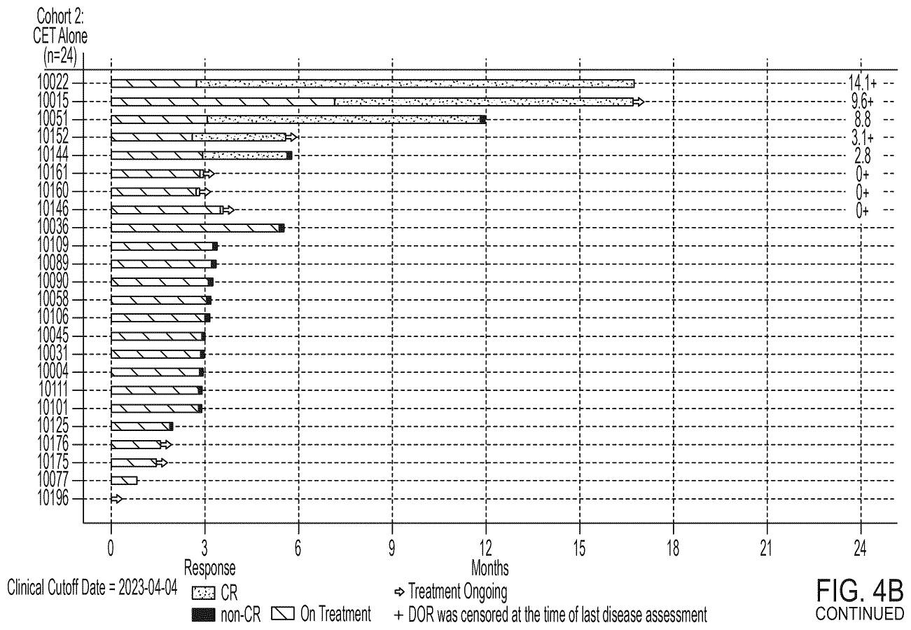

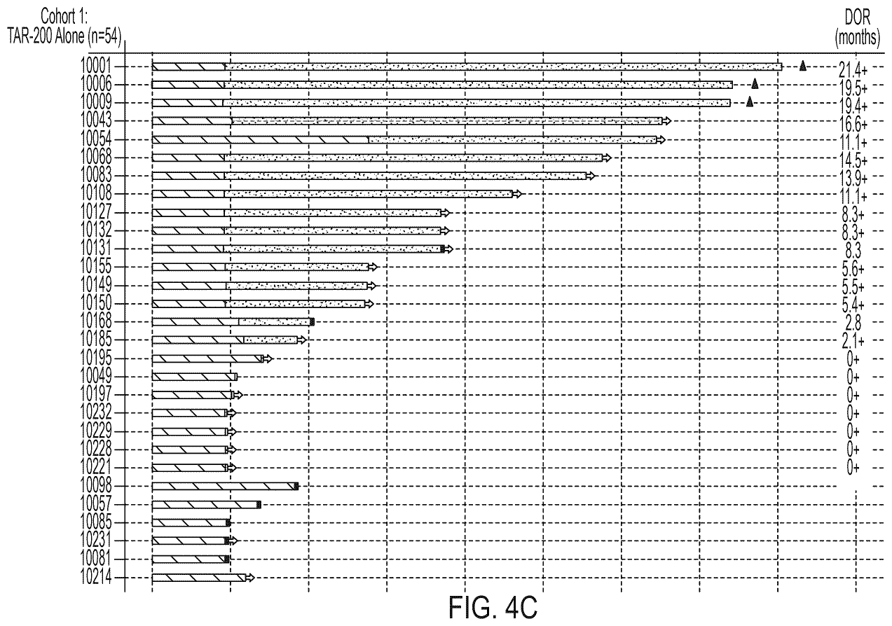

In the evaluable analysis set, there were 19 participants in cohort 1, and 18 participants in cohort 2. There were 15, and 6 complete responses, respectively, in the 2 cohorts. The overall CR rate and corresponding 95% confidence interval (CI) was 78.9% (54.4%, 93.9%) in cohort 1, and 33.3% (13.3%, 59.0%) in cohort 2. The median duration of response was not estimable for either cohort. Among the responders, no participants had an event in cohort 1, and 1 had an event (16.7%) in cohort 2 (Table 2, FIG. 4A).

The overall CR rate (95% CI) based on investigator was 84.2% (60.4%, 96.6%) in cohort 1, and 44.4% (21.5%, 69.2%) in cohort 2. The overall concordance rate between central and investigator assessment was 82.9%. Forty-four of the 45 overall CRs (97.8%) confirmed by central were also assessed as overall CR by investigator and the other 1 was missing investigator assessment.

TABLE 2
Summary of Best Overall response by
Central Review; Treated Analysis Set

| | Cohort 1 | | Cohort 2 | |
|---|---|---|---|---|
| | TAR-200 | | CET | |
| Analysis set: Treated | 22 | | 23 | |
| Evaluable^{a} | 19 | (86.4%) | 18 | (78.3%) |
| Non-Evaluable | 3 | (13.6%) | 5 | (21.7%) |
| Best overall response | | | | |
| Complete response (CR)^{b} | 15 | (78.9%) | 6 | (33.3%) |
| 95% CI^{c} | (54.4%, 93.9%) | | (13.3%, 59.0%) | |
| Non-complete response (Non-CR) | 4 | (21.1%) | 12 | (66.7%) |

Note:
Response is based on centrally reviewed urine cytology, local cystoscopy, and central biopsy (if available). CR does not have to be confirmed.
Note:
Percentages for response rates are calculated based on the number of evaluable subjects.
^{a}Evaluable Analysis Set is defined as all treated participants who have active disease at baseline and adequate disease assessment post-baseline, or who have progressed, died, been withdrawn from study due to recurrence of high-risk disease or progressive disease.
^{b}A Complete Response is defined as having a negative central cytology and negative central biopsy, if available.
^{c}95% CI is estimated using the exact method based on binomial distribution.
Clinical Cutoff Date = 20 Feb. 2023

### Safety

Overall, 21 (95.5%) participants in cohort 1, and 18 (78.3%) participants in cohort 2 experienced at least one treatment-emergent adverse event (TEAE) during the reporting period. The most common TEAEs (at least in 20%) were as follows: Cohort 1: micturition urgency (36.4%), pollakiuria (36.4%), cystitis noninfective (22.7%), dysuria (22.7%), and urinary tract infection (22.7%), Cohort 2: pruritus (21.7%).

Table 3, provides the numbers of subjects who experienced TEAEs, grade 3 or higher TEAEs, serious TEAEs, TEAEs leading to treatment discontinuation, TEAEs with fatal outcome, and the associated relationships to study treatment, procedure or device.

Grade 3 or higher TEAEs occurred in 4 (18.2%) participants in cohort 1, and 2 (8.7%) in cohort 2. Most of these TEAEs were reported in only 1 participant and approximately half were not related to study treatment per investigator assessment.

One participant experienced a serious TEAE (4.5%) in cohort 1, and one participant experienced a serious TEAE (4.3%) in cohort 2. None of these serious TEAEs were reported in more than 1 participant and the majority were not related to study treatment per investigator assessment.

Immune related TEAEs (irAEs) as reported by the investigator were experienced by 10 (43.5%) participants in cohort 2. The most common irAE (at least in 10%) was pruritus in 3 participants (13.0%) in cohort 2. Grade 3 or higher irAEs were reported in 6 (14.0%) and 2 (8.7%) participants in cohort 1 and 3, respectively. Five of 10 participants (50.0%) in cohort 2 who experienced an irAE required treatment with a steroid. For those events with toxicity grade 3 or higher, steroid was used in 1 (1 oral) participant in cohort 2. The median time for those grade 3 or higher irAEs to be resolved or improved to grade 51.5 days in cohort 2.

There were 0 deaths during the study in the reporting period.

There were 4 (18.2%) participants who experienced TEAEs leading to discontinuation of TAR-200 in cohort 1. Most TEAEs were Grade≤2 and were associated with lower urinary tract symptoms. These TEAEs are similar to those historically experienced with intravesical BCG, with up to 60% of patients discontinuing BCG in published series due to Grade 1 AEs (Serretta, Vincenzo et al. Urologia internationalis vol. 96, 1 (2016): 20-4.). There was 1 (4.3%) participant who experienced TEAEs leading to discontinuation of cetrelimab in cohort 2.

Based on the Sponsor's early experience to date and as the study continues to enroll, several specific mitigations have been put in place to ameliorate these issues. The allowed time to re-exposure was extended in patients recovering from an AE beyond 3 weeks was added to the protocol via a recent global amendment (Amendment 3). This amendment affords patients more than 3 weeks from the onset of symptoms to resolve these symptoms to <Grade 2 prior to subsequent redosing. Post placement cystoscopy is also recommended if there is any concern that the intravesical drug delivery system may not be optimally in place within the bladder lumen following the insertion procedure. Patients have further been re-advised to hydrate liberally while on study. Lastly, dosing should not be performed in any patient with signs of an active or evolving UTI, even in those with urine culture CFUs <105. These measures have already materially resulted in a lower frequency of early TAR-200 discontinuations as the study has continued to enroll globally.

TABLE 3
Overall Summary of Treatment-emergent Adverse Events; Treated Analysis Set

| | Cohort 1 | Cohort 2 | |
|---|---|---|---|
| | TAR-200 | CET | Overall |
| Analysis set: Treated | 22 | 23 | 45 |
| Subjects with 1 or more: | | | |
| TEAEs | 21 (95.5%) | 18 (78.3%) | 39 (86.7%) |
| Related TEAEs^{a} | 16 (72.7%) | 10 (43.5%) | 26 (57.8%) |
| TAR-200 | 15 (68.2%) | 0 | 15 (33.3%) |
| Cetrelimab | 0 | 10 (43.5%) | 10 (22.2%) |
| Insertion Procedure | 4 (18.2%) | 0 | 4 (8.9%) |
| Removal Procedure | 1 (4.5%) | 0 | 1 (2.2%) |
| Urinary Placement Catheter | 3 (13.6%) | 0 | 3 (6.7%) |
| Grade >=3 TEAEs | 4 (18.2%) | 2 (8.7%) | 6 (13.3%) |
| Grade >=3 Related TEAEs^{a} | 0 | 2 (8.7%) | 2 (4.4%) |
| TAR-200 | 0 | 0 | 0 |
| Cetrelimab | 0 | 2 (8.7%) | 2 (4.4%) |
| Insertion Procedure | 0 | 0 | 0 |
| Removal Procedure | 0 | 0 | 0 |
| Urinary Placement Catheter | 0 | 0 | 0 |
| Serious TEAEs | 1 (4.5%) | 1 (4.3%) | 2 (4.4%) |
| Related Serious TEAEs^{a} | 0 | 1 (4.3%) | 1 (2.2%) |
| TAR-200 | 0 | 0 | 0 |
| Cetrelimab | 0 | 1 (4.3%) | 1 (2.2%) |
| Insertion Procedure | 0 | 0 | 0 |
| Removal Procedure | 0 | 0 | 0 |
| Urinary Placement Catheter | 0 | 0 | 0 |
| TEAEs Leading to Treatment Discontinuation | 4 (18.2%) | 1 (4.3%) | 5 (11.1%) |
| Related TEAEs Leading to Treatment | 2 (9.1%) | 1 (4.3%) | 3 (6.7%) |
| Discontinuation | | | |
| TAR-200 | 1 (4.5%) | 0 | 1 (2.2%) |
| Cetrelimab | 0 | 1 (4.3%) | 1 (2.2%) |
| Insertion Procedure | 1 (4.5%) | 0 | 1 (2.2%) |
| Removal Procedure | 0 | 0 | 0 |
| Urinary Placement Catheter | 0 | 0 | 0 |
| TEAEs with Fatal Outcome^{c} | 0 | 0 | 0 |
| Related TEAEs with Fatal Outcome^{a, c} | 0 | 0 | 0 |
| TAR-200 | 0 | 0 | 0 |
| Cetrelimab | 0 | 0 | 0 |
| Insertion Procedure | 0 | 0 | 0 |
| Removal Procedure | 0 | 0 | 0 |
| Urinary Placement Catheter | 0 | 0 | 0 |
| COVID-19 associated TEAEs^{d} | 3 (13.6%) | 4 (17.4%) | 7 (15.6%) |
| COVID-19 associated Serious TEAEs^{d} | 0 | 0 | 0 |
| COVID-19 associated Non-serious TEAEs^{d} | 3 (13.6%) | 4 (17.4%) | 7 (15.6%) |

Key: AE = adverse event; TEAE = treatment emergent adverse event; CET = cetrelimab
^{a}An AE is categorized as related if the investigator determines that there is a possible, or causal relationship between the AE and insertion or removal procedure or UPC.
^{b}Number of subjects who experienced AEs related to TAR-200, insertion procedure, removal procedure, or UPC which led to discontinuation of TAR-200 or those who experienced cetrelimab related AEs which led to discontinuation of cetrelimab.
^{c}Based on the TEAEs with outcome assessed as Fatal, irrespective of the grade.
^{d}COVID-19 associated AEs are based on events that code to a COVID-19 medDRA term and events that are identified via the COVID-19 Case of AEs form.
Subjects are counted only once for any given event, regardless of the number of times they actually experienced the event.

### CONCLUSION

This analysis included data from a total of 45 patients randomized and treated in Cohort 1 or Cohort 2. Review of efficacy data demonstrated robust CR rates for Cohort 1 by central review of 78.9, and with the lower bound of 95% confidence interval higher than 50% in Cohorts 1 and 2. The median DOR is not reached with approximately 9 months of median follow up. Safety data for TAR-200 alone, and cetrelimab alone demonstrated an acceptable safety and tolerability profile. Immune related adverse events were consistent with the known safety profile of anti-PD-1 antibodies and were manageable.

Thus, following completion of the clinical work, the conclusion was that the data showed a striking 78.9% CR rate for monotherapy comprising local and continuous administration of a demonstrated clinically effective amount of gemcitabine to the bladder. This represents a significant advance in treatment of individuals with HR-NMIBC over the currently available therapies and makes this therapy a viable alternative to radical cystectomy for these high-risk patients.

### Example 2

This study was an open-label, parallel-group, multi-center study evaluating the efficacy and safety of intravesical TAR-200 alone, systemic intravenous (IV) cetrelimab alone, or intravesical TAR-200 in combination with systemic IV cetrelimab in participants with HR NMIBC (CIS with or without papillary disease) unresponsive to prior intravesical BCG therapy, who are either ineligible for or have elected not to undergo RC after making an informed decision. Participants were randomly assigned (1:1:2) to receive intravesical-TAR-200 alone (Cohort 1), IV cetrelimab alone (Cohort 2), or intravesical TAR-200 in combination with systemic IV cetrelimab (Cohort 3). The amounts provided were hoped to be clinically effective amounts, however analysis of the data generated during this study would be required to conclude that the endpoints were met.

For Cohort 1, TAR-200 alone was dosed every 3 weeks (Q3W) for up to the first 24 weeks (6 months) on study, as shown in FIG. 3A. Dosing of TAR-200 then occurred every 12 weeks through Week 99 (Year 2, as shown in FIG. 3B). Each TAR-200 was removed after a 3-week dwell time through Week 99 (Year 2). For Cohort 2, cetrelimab was dosed at 360 mg IV Q3W through Week 78 (18 months), as shown in FIGS. 3A and 3B. For Cohort 3, TAR-200 was dosed Q3W for up to the first 24 weeks (6 months), as shown in FIG. 3A. Dosing of TAR-200 then occurred every 12 weeks through Week 99 (Year 2, as shown in FIG. 3B). Each TAR-200 was removed after a 3-week dwell time through Week 99 (Year 2). Cetrelimab was dosed at 360 mg IV Q3W through Week 78 (18 months). Dosing of TAR-200 then occurred every 12 weeks through Week 99 (Year 2).

The following analysis was performed on updated data from patients in Cohort 1 and Cohort 2 treated through 4 Apr. 2023 (clinical cut-off date). Results for the TAR-200 monotherapy arm and Cetrilimab monotherapy arm are provided.

In the safety analysis (n=23), 16 (69.6%) patients are ongoing at clinical cutoff with TAR-200 and 7 (30.4%) patients discontinued treatment. Among the 7 patients with TAR-200 discontinuation, the reason for discontinuation was persistent disease in 2 patients, adverse event in 2 patients, subject refused further treatment in 2 patients, and physician decision in 1 patient.

In the safety analysis set, 16 (69.6%) patients were White, 19 (82.6%) patients were male, and the median (range) age was 72.0 (40; 81) years. Twenty-two (95.7%) patients elected to not undergo RC. The primary reasons for refusal included preservation of bladder in 13 patients and concern about quality of life after cystectomy in 7 patients. At baseline, 16 (69.6%) patients had carcinoma in situ (CIS) only and 7 (30.4%) patients had CIS with concurrent Ta disease. All patients had adequate prior BCG therapy as defined by the FDA Guidance for Industry. Baseline characteristics, including information on prior BCG therapy are summarized below (Table 4, and Table 5A, 5B).

TABLE 4
Patient Population Demographics in TAR-
200 and Cetrelimab Monotherapy Groups

| | TAR-200 | Cetrelimab |
|---|---|---|
| Characteristics | (N = 23) | (N = 24) |
| Age, years, median (range) | 72.0 (40-81) | 70.0 (51-88) |
| Sex, male, % | 82.6 | 79.2 |
| Race, % | | |
| White | 69.6 | 83.3 |
| Asian | 0 | 4.2 |
| Black or African American | 4.3 | 0 |
| Not reported | 26.1 | 8.3 |
| Unknown | 0 | 4.2 |
| ECOG performance status 0, % | 95.7 | 95.8 |

TABLE 5A
Patient Population Baseline Characteristics
in TAR-200 and Cetrelimab Monotherapy Groups

| | TAR-200 | Cetrelimab |
|---|---|---|
| Characteristics | (N = 23) | (N = 24) |
| Tumor stage, %^{a} | | |
| CIS only | 69.6 | 65.2 |
| CIS + papillary disease | 30.4 | 34.7 |
| Total doses of prior BCG, n, median (range) | 12 (7-24) | 12 (7-30) |
| Time from last BCG to CIS diagnosis, | 3.0 (1-22) | 3.1 (0-11) |
| months, median (range) | | |
| Reason for not receiving RC, % | | |
| Declined | 95.7 | 100 |
| Ineligible | 4.3 | 0 |

^{a}Stages are mutually exclusive. Urothelial carcinoma in situ includes patients
with CIS only. CIS + pTa includes patients who have both CIS and pTa.
CIS + pT1 includes patients with CIS and pT1 or CIS, pTa, and pT1.
ECOG, Eastern Cooperative Oncology Group; RC, radical cystectomy.

TABLE 5B
Detailed summary of NMIBC Baseline Disease Characteristics
in the TAR-200 Monotherapy Group

TAR-200

| Analysis set: Safety | 23 | |
|---|---|---|
| Baseline ECOG | | |
| N | 23 | |
| 0 | 22 | (95.7%) |
| 1 | 1 | (4.3%) |
| 2 | 0 | |
| Tumor Stage^{a} | | |
| N | 23 | |
| Carcinoma in situ only | 16 | (69.6%) |
| CIS + pTa | 7 | (30.4%) |
| CIS + pT1 | 0 | |
| Number of Prior Intravesical BCG Doses | | |
| N | 23 | |
| Mean (SD) | 13.5 | (4.89) |
| Median | 12.0 | |
| Range | (7; 24) | |
| Number of Prior Intravesical BCG Doses | | |
| N | 23 | |
| <7 | 0 | |
| 7-9 | 7 | (30.4%) |
| 10-14 | 7 | (30.4%) |
| 15-19 | 6 | (26.1%) |
| 20+ | 3 | (13.0%) |
| Time from most recent BCG dose to diagnosis | | |
| of BCG unresponsive disease (months) | | |
| N | 22 | |
| Mean (SD) | 4.2 | (4.59) |
| Median | 3.0 | |
| Range | (1; 22) | |
| Reason for not receiving RC | | |
| N | 23 | |
| Ineligible | 1 | (4.3%) |
| Medical and surgical comorbidities | 1 | (4.3%) |
| Age | 0 | |
| Elected Not to Undergo | 22 | (95.7%) |
| Preservation of bladder | 13 | (56.5%) |
| Concern about quality of life after | 7 | (30.4%) |
| cystectomy | | |
| Concern about mortality & morbidity | 1 | (4.3%) |
| risk associated with procedure | | |
| Preservation sexual function | 1 | (4.3%) |

Key: RC = radical cystectomy
^{a}Stages are mutually exclusive. CIS + pT1 includes subjects with CIS and pT1 or CIS, pTa, and pT1.

The preliminary efficacy assessment was performed on the efficacy analysis set in the TAR-200 monotherapy cohort (n=22) (Table 6). The analysis set includes 20 patients with at least 1 post-baseline disease assessment and 2 non-evaluable patients who discontinued from study with no post-baseline assessment. The analysis showed a robust overall CR rate of 72.7% (95% CI: 49.8%, 89.3%), with 16 patients achieving CR, 4 patients achieving non-CR, and 2 patients discontinued from study with no post-baseline assessment.

The overall CR rate (95% CI) based on investigator assessment was 77.3% (95% CI: 54.6%, 92.2%) in TAR-200 monotherapy cohort. The overall concordance rate between central and investigator assessment was 95.5%. Of the 17 CRs by investigator, 16 CRs were confirmed by central assessment.

At the clinical cut-off date of 4 Apr. 2023, the median DOR was not reached after a median of 10.6 months follow-up, with 15 out of 16 responders having achieved CR at first disease assessment at week 12. Further, 15 out of 16 patients with documented CR continue to be on treatment with ongoing CR. In the efficacy analysis set (n=22), 10 (45.5%) patients had an ongoing CR at 6 months, 7 (31.8%) patients had an ongoing CR at 12 months, and 3 (13.6%) patients had an ongoing CR at 18 months. Only 1 out of 16 patients discontinued treatment 46 days after achieving complete response due to physician decision and refused further follow-up. None of the patients with documented CR had a reported recurrence or progression event. None of the patients in the analysis set had documented progression to muscle invasive or metastatic disease and no death events were reported.

It is important to note that the CR rate of 72.7% (95% CI: 49.8%, 89.3%) with TAR-200 monotherapy based on central review was achieved in accordance with FDA guidance for industry on BCG unresponsive NMIBC. The definition of CR is consistent with the FDA's definition; therefore, patients with positive urine cytology and negative tissue biopsy or patients with negative urine cytology and non-evaluable cystoscopy were not considered to have a CR. Hence, 15 out of 16 patients achieved CR at first disease assessment at week 12 and 4 patients with non-CR discontinued prior to month 6 (week 24).

TABLE 6
Summary of Best Overall response by
Central Review; Treated Analysis Set

| | Cohort 1 | Cohort 2 |
|---|---|---|
| | TAR-200 | CET |
| Analysis set: Efficacy | 22 | 21 |
| Best overall response | | |
| Complete response (CR) | 16 (72.7%) | 8 (38.1%) |
| 95% CI^{a} | (49.8%, 89.3%) | (18.1%, 61.6%) |
| Non-complete response (Non-CR) | 6 (27.3%) | 13 (61.9%) |
| Non-evaluable | 2 (9.1%) | 1 (4.8%) |

Note:
Response is based on centrally reviewed urine cytology, local cystoscopy, and central biopsy (if available). CR does not have to be confirmed.
Note:
Percentages for response rates are calculated based on the number of evaluable subjects.
^{a}95% CI is estimated using the exact method based on binomial distribution.
Clinical Cutoff Date = 2023 Apr. 4

The preliminary safety assessment includes data on a total of 23 patients with TAR-200 monotherapy with a median follow-up of 10.6 months (0; 23) (Table 7). Overall, 21 (91.3%) patients in TAR-200 monotherapy experienced at least one treatment-emergent adverse event (TEAE) during the reporting period. Micturition urgency (34.8%), pollakiuria (34.8%), dysuria (23.4%), cystitis noninfective (21.7%), and urinary tract infection (21.7%) were the most commonly reported TEAEs (at least 20%) and were mostly Grade 1-2 events that were manageable with symptomatic therapy (Table 8).

TEAEs ≥G3 were reported in 7 (30.4%) patients, two of which were related to TAR-200 monotherapy. The rate of TEAEs ≥G3 is not unexpected in this elderly and frail patient population. Three serious TEAEs were reported in this cohort: cystitis noninfective and urinary tract infection each in 1 patient which resolved and were deemed unrelated by the investigator, renal impairment and urosepsis in 1 patient which resolved and was deemed related by the investigator. Three (13.0%) patients had TEAEs that led to treatment discontinuation, however, only 2 (8.7%) patients had TAR-200 or insertion procedure related TEAEs leading to discontinuation (cystitis noninfective in each patient).

TABLE 7
Overall Summary of Treatment-emergent Adverse Events; Treated Analysis Set

| | Cohort 1 | Cohort 2 | |
|---|---|---|---|
| | TAR-200 | CET | Overall |
| Analysis set: Safety | 23 | 24 | 47 |
| Subjects with 1 or more: | | | |
| TEAEs | 21 (91.3%) | 19 (79.2%) | 40 (85.1%) |
| Related TEAEs^{a} | 17 (73.9%) | 12 (50.0%) | 29 (61.7%) |
| TAR-200 | 15 (65.2%) | 0 | 15 (31.9%) |
| Cetrelimab | 0 | 12 (50.0%) | 12 (25.5%) |
| Insertion Procedure | 5 (21.7%) | 0 | 5 (10.6%) |
| Removal Procedure | 2 (8.7%) | 0 | 2 (4.3%) |
| Urinary Placement Catheter | 4 (17.4%) | 0 | 4 (8.5%) |
| Grade >=3 TEAEs | 7 (30.4%) | 1 (4.2%) | 8 (17.0%) |
| Grade >=3 Related TEAEs^{a} | 2 (8.7%) | 1 (4.2%) | 3 (6.4%) |
| TAR-200 | 2 (8.7%) | 0 | 2 (4.3%) |
| Cetrelimab | 0 | 1 (4.2%) | 1 (2.1%) |
| Insertion Procedure | 0 | 0 | 0 |
| Removal Procedure | 0 | 0 | 0 |
| Urinary Placement Catheter | 0 | 0 | 0 |
| Serious TEAEs | 3 (13.0%) | 2 (8.3%) | 5 (10.6%) |
| Related Serious TEAEs^{a} | 1 (4.3%) | 2 (8.3%) | 3 (6.4%) |
| TAR-200 | 1 (4.3%) | 0 | 1 (2.1%) |
| Cetrelimab | 0 | 2 (8.3%) | 2 (4.3%) |
| Insertion Procedure | 0 | 0 | 0 |
| Removal Procedure | 0 | 0 | 0 |
| Urinary Placement Catheter | 0 | 0 | 0 |
| TEAEs Leading to Treatment Discontinuation | 3 (13.0%) | 1 (4.2%) | 4 (8.5%) |
| Related TEAEs Leading to Treatment | 2 (8.7%) | 1 (4.2%) | 3 (6.4%) |
| Discontinuation | | | |
| TAR-200 | 1 (4.3%) | 0 | 1 (2.1%) |
| Cetrelimab | 0 | 1 (4.2%) | 1 (2.1%) |
| Insertion Procedure | 1 (4.3%) | 0 | 1 (2.1%) |
| Removal Procedure | 0 | 0 | 0 |
| Urinary Placement Catheter | 0 | 0 | 0 |
| TEAEs with Fatal Outcome^{c} | 0 | 0 | 0 |
| Related TEAEs with Fatal Outcome^{a, c} | 0 | 0 | 0 |
| TAR-200 | 0 | 0 | 0 |
| Cetrelimab | 0 | 0 | 0 |
| Insertion Procedure | 0 | 0 | 0 |
| Removal Procedure | 0 | 0 | 0 |
| Urinary Placement Catheter | 0 | 0 | 0 |
| Immune-related | 0 | 12 (50.0%) | 12 (25.5%) |
| Grade 3 or higher Immune-related | 0 | 2 (8.3%) | 2 (4.3%) |

Key: AE = adverse event; TEAE = treatment emergent adverse event; CET = cetrelimab
^{a}An AE is categorized as related if the investigator determines that there is a possible, or causal relationship between the AE and insertion or removal procedure or UPC.
^{b}Number of subjects who experienced AEs related to TAR-200, insertion procedure, removal procedure, or UPC which led to discontinuation of TAR-200 or those who experienced cetrelimab related AEs which led to discontinuation of cetrelimab.
^{c}Based on the TEAEs with outcome assessed as Fatal, irrespective of the grade.
^{d}COVID-19 associated AEs are based on events that code to a COVID-19 medDRA term and events that are identified via the COVID-19 Case of AEs form.
Subjects are counted only once for any given event, regardless of the number of times they actually experienced the event.

TABLE 8
Overall Safety Profile of TAR-200 and Cetrelimab Monotherapy Groups

| | Cohort 1: TAR-200 | | Cohort 2: CET | |
|---|---|---|---|---|
| Patients with events, n (%)^{a} | Any grade | Grade ≥3 | Any grade | Grade ≥3 |
| ≥1 AE | 21 (91.3) | 7 (30.4) | 19 (79.2) | 2 (8.3) |
| Micturition urgency | 8 (34.8) | 0 | 1 (4.2) | 0 |
| Pollakiuria | 8 (34.8) | 0 | 0 | 0 |
| Dysuria | 6 (26.1) | 0 | 1 (4.2) | 0 |
| Noninfective cystitis | 5 (21.7) | 1 (4.3) | 0 | 0 |
| Urinary tract infection | 5 (21.7) | 1 (4.3) | 4 (16.7) | 0 |
| Pruritus | 0 | 0 | 5 (20.8) | 0 |
| Diarrhea | 0 | 0 | 5 (20.8) | 0 |

^{a}AE by preferred term in ≥20% of patients.
AE, adverse event; SAE, serious adverse event.

### SUMMARY

BCG unresponsive HR-NMIBC is unanimously regarded as a life-threatening malignancy with a significant risk of progression. Disease progression to MIBC is invariably associated with significant patient morbidity and mortality. RC, the standard of care for BCG unresponsive HR-NMIBC, is associated with significant morbidity and an impact on the quality of life, and many patients are unfit or unwilling to undergo this radical, extirpative surgery. Bladder-preserving options for patients with BCG unresponsive HR-NMIBC remain limited and associated with significant shortcomings such as systemic and immune-related toxicities, limited durability of response or the reliance on BCG for combination therapies. Therefore, there remains a high unmet need for bladder-sparing, BCG-free and localized therapies with durable responses and an acceptable safety profile.

The preliminary clinical evidence shows that monotherapy comprising local and continuous administration of a clinically effective amount of gemcitabine, as proven by the data, to the bladder has meaningful activity in patients with HR-NMIBC, with a robust CR rate of 72.7% (95% CI: 49.8%, 89.3%). Results show that responses to TAR-200 are durable, with the median DOR not reached after a median of 10.6 months of follow-up. Out of 16 complete responses to TAR-200, 15 were ongoing at the clinical cut-off date for this analysis. No disease recurrence or progression were reported in any of the 16 responders to TAR-200 monotherapy. No death events were reported in any of the 23 patients in the safety analysis set. Treatment with TAR-200 was well tolerated with mostly low-grade and manageable irritative urinary symptoms such as dysuria, pollakiuria, and nocturia. There were 2 (8.7%) treatment-related Grade >=3 TEAEs, 1 (4.3%) treatment-related serious TEAE, and 2 (8.7%) patients with treatment-related TEAEs leading to discontinuation.

TAR-200 represents an innovative advancement in the treatment of urothelial carcinoma of the urinary bladder, wherein it provides a potentially effective treatment with acceptable bladder tolerability and minimal systemic exposure. Such features may avoid the known off-target toxicities of gemcitabine, including marrow suppression, as well as the significant lower urinary tract symptoms associated with frequent, high-dose intravesical instillations. Collectively, these preliminary clinical data with TAR-200 monotherapy demonstrate robust and durable anti-tumor activity in this population where there is a high unmet medical need. Thus, local administration of gemcitabine to the bladder over a period of time, such as about 24 weeks has the potential to offer substantial benefit in the treatment of this serious and life-threatening condition over available treatment options.

### Example 3

The following analysis of the study described in Examples 1 and 2 was performed on updated data from patients in Cohort 1 treated through 24 Aug. 2023 (clinical cut-off date). Results for the TAR-200 monotherapy arm are provided.

### Background:

Patients with BCG-unresponsive HR-NMIBC are at high risk of disease progression with limited treatment (tx) options. TAR-200, an intravesical drug delivery system, provides sustained release of gemcitabine in the bladder over many days. SunRISe-1 (NCT04640623) is an ongoing, randomized, phase 2b study assessing efficacy and safety of TAR-200 alone (C1), or cetrelimab alone (C2) in patients with BCG-unresponsive HR NMIBC ineligible for or refusing radical cystectomy. We report results from C1.

### Methods:

Eligible patients aged ≥18 years old had histologically confirmed carcinoma in situ (CIS)±papillary disease (high-grade Ta, any T1), ECOG performance status 0-2, and persistent or recurrent HR NMIBC with last dose of BCG ≤12 months prior to CIS diagnosis. TAR-200 was dosed Q3W through week 24, then Q12W until week 96. Response assessments: cystoscopy and centrally assessed urine cytology, CT/MRI, and bladder biopsy (at weeks 24, 48, and as clinically indicated). Primary end point: overall complete response (CR) rate. Secondary end points: duration of response (DOR), overall survival, safety, and tolerability.

### Results:

At 24 Aug. 2023 data cutoff, 54 patients (median age, 71 years; range, 40-85; 33% with concurrent papillary disease) received TAR-200 monotherapy. 30 patients were efficacy evaluable. Centrally confirmed CR by urine cytology and/or biopsy was achieved in 23/30 patients (77%; 95% CI, 58-90). Median DOR was not reached (median follow-up in responders, 48 weeks; range 12-121); 21/23 responders remain in CR with 11/11 and 6/6 having response ≥6 months and ≥12 months from CR, respectively. CR rate by investigator assessment was comparable with central results. 29 patients (54%) had treatment-related adverse events (TRAEs); most common (≥10%) were pollakiuria, dysuria, and micturition urgency. 4 patients (7%) had Gr ≥3 TRAEs; 1 (2%) had serious TRAE; 2 (4%) had TRAEs leading to discontinuation. No deaths were reported.

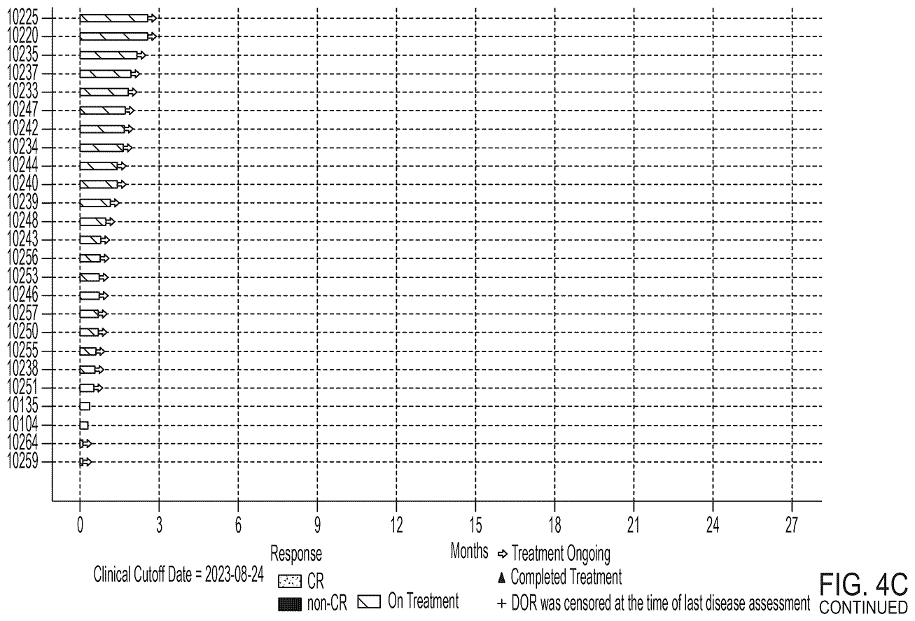

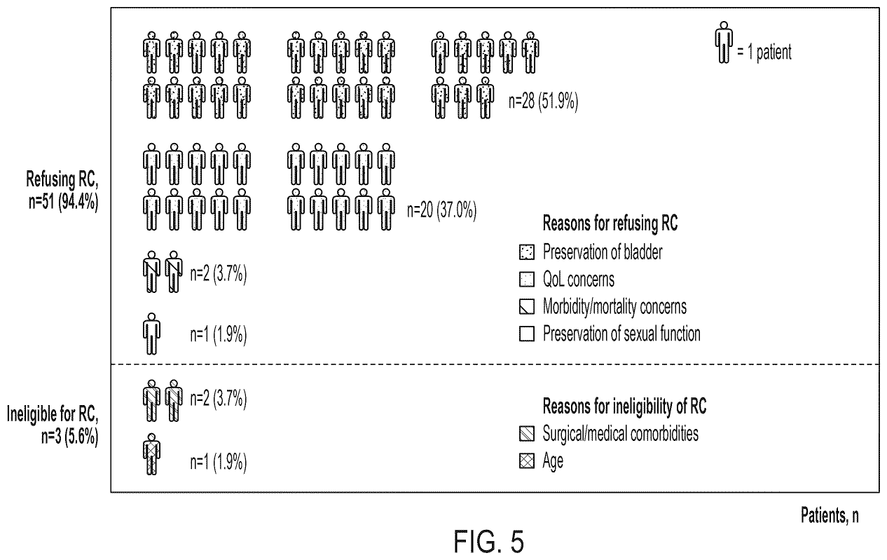

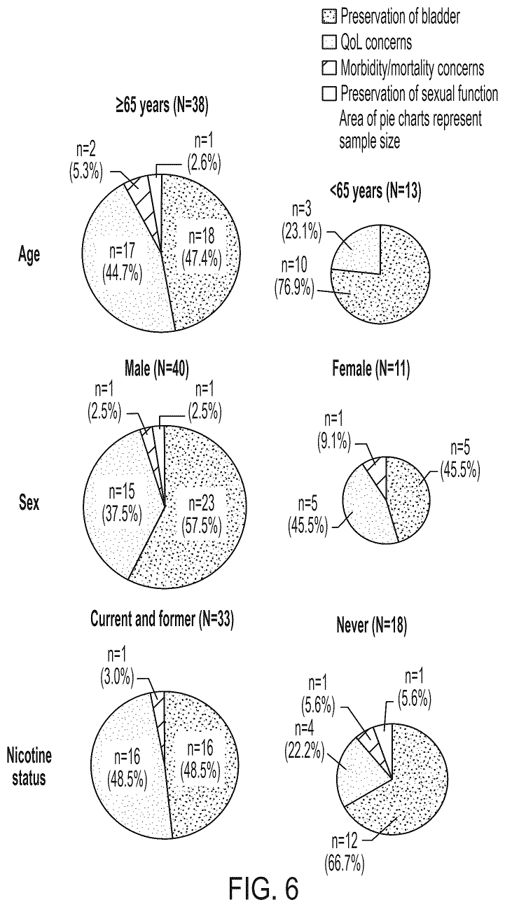

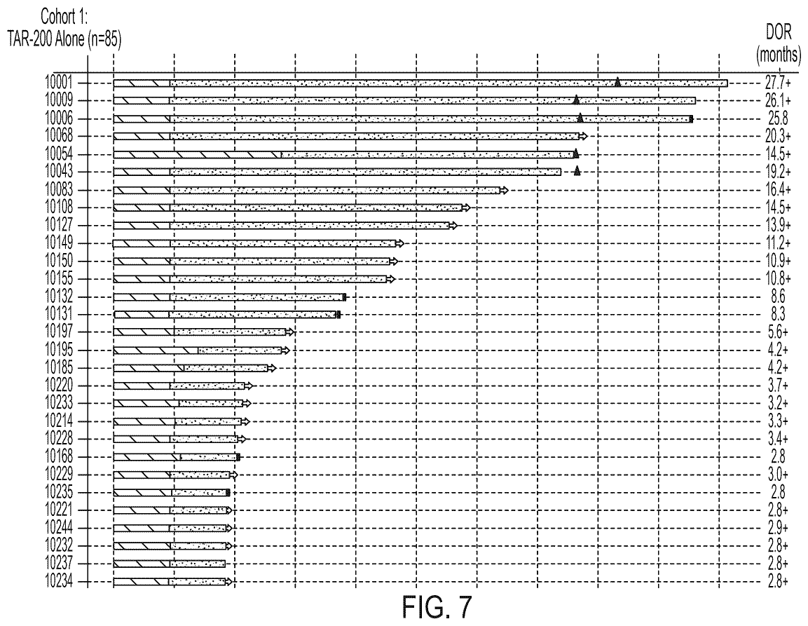

Baseline characteristics, including information on prior BCG therapy are summarized below (Tables 9A, 9B, and 9C). Further nicotine use history data were obtained and are summarized in Table 9A2. Overall, more than 94% of patients refused RC and only 5.6% of patients were ineligible for RC (Table 9B, FIG. 5). Of those who refused RC, 51.9% of patients cited the preservation of their bladder as the primary reason for refusal, while 37% of patients cited quality of life concerns. The reasons patients were ineligible for RC included surgical and medical comorbidities (3.7% of patients) and age (1.9% of patients). Across age, gender, and nicotine use, most patients refused RC to preserve the bladder (FIG. 6). Patients at or over the age of 65 cited the preservation of their bladder and quality of life concerns as the primary reasons to refuse RC at similar rates (47.4% and 44.7%, respectively), while patients under the age of 65 predominantly cited the preservation of their bladder as the primary reason to refuse RC (76.9% of patients). Across sex, 57.5% of male patients cited the preservation of their bladder as the primary reason to refuse RC, while the primary reason to refuse RC in female patients was divided between the preservation of their bladder and quality of life concerns equally (45.5% for each). Current and former nicotine users cited the preservation of their bladder (48.5% of patients) and quality of life concerns (48.5% of patients) as the primary reasons to refuse RC equally. The majority of patients who have never used nicotine refused RC to preserve their bladder (66.7% of patients). Moreover, no responders underwent RC as of the data cutoff (n=23 of 23). Of the 7 non-responders, only one patient who was a non-responder at week 12 went on to receive RC.

TABLE 9A
Summary of Demographics and Baseline Characteristics
for TAR-200 (Cohort 1); Treated Analysis Set

| | Cohort 1 |
|---|---|
| | TAR-200 |
| Analysis set: Full | 54 |
| Age, (years) | |
| N | 54 |
| Mean (SD) | 69.7 (8.81) |
| Median | 71.0 |
| Range | (40; 85) |
| <65 | 13 (24.1%) |
| 65-74 | 23 (42.6%) |
| 75-84 | 16 (29.6%) |
| >=85 | 2 (3.7%) |
| Sex | |
| N | 54 |
| Female | 12 (22.2%) |
| Male | 42 (77.8%) |
| Undifferentiated | 0 |
| Unknown | 0 |
| Race^{a} | |
| N | 54 |
| American Indian or Alaska Native | 0 |
| Asian | 4 (7.4%) |
| Black or African American | 2 (3.7%) |
| Native Hawaiian or Other Pacific | 0 |
| Islander | |
| White | 37 (68.5%) |
| Not Reported | 11 (20.4%) |
| Unknown | 0 |
| Ethnicity | |
| N | 54 |
| Hispanic or Latino | 1 (1.9%) |
| Not Hispanic or Latino | 40 (74.1%) |
| Not Reported | 11 (20.4%) |
| Unknown | 2 (3.7%) |
| Region^{b} | |
| N | 54 |
| America | 14 (25.9%) |
| Asia | 6 (11.1%) |
| Eastern Europe | 0 |
| Western Europe | 34 (63.0%) |
| Nicotine Use History | |
| N | 54 |
| Current | 5 (9.3%) |
| Former | 30 (55.6%) |
| Never | 19 (35.2%) |

Note:
N's for each parameter reflect non-missing values.
^{a}If multiple Race categories are indicated, the Race is recorded as “Multiple”.
^{b}America includes Canada, USA; Asia includes Australia, Japan, South Korea; Eastern Europe includes Russia, Turkey, Ukraine; Western Europe includes Belgium, France, Germany, Greece, Italy, Netherlands, Portugal, Spain, UK.
Clinical Cutoff Date = 24 Aug. 2023.

TABLE 9A2
Updated Nicotine Use History (Cohort 1); Treated Analysis Set

| Nicotine Use History | |
|---|---|
| N | 54 |
| Current | 5 (9.3%) |
| Former | 28 (51.9%) |
| Never | 18 (33.3%) |

TABLE 9B
Summary of NMIBC Baseline Disease Characteristics
for TAR-200 (Cohort 1); Treated Analysis Set

| | Cohort 1 | |
|---|---|---|
| | TAR-200 | |
| Analysis set: Full | 54 | |
| Baseline ECOG | | |
| N | 54 | |
| 0 | 52 | (96.3%) |
| 1 | 2 | (3.7%) |
| 2 | 0 | |
| Tumor Stage^{a} | | |
| N | 54 | |
| Urothelial carcinoma in situ | 36 | (66.7%) |
| CIS + pTa | 14 | (25.9%) |
| CIS + pT1 | 4 | (7.4%) |
| Papillary Disease (pTa, pT1) | | |
| N | 54 | |
| Present | 18 | (33.3%) |
| Absent | 36 | (66.7%) |
| Time from most recent BCG dose to | | |
| diagnosis of BCG unresponsive | | |
| disease (months) | | |
| N | 54 | |
| Mean (SD) | 3.9 | (3.58) |
| Median | 3.0 | |
| Range | (0; 22) | |
| Reason for not receiving RC | | |
| N | 54 | |
| Ineligible | 3 | (5.6%) |
| Age | 1 | (1.9%) |
| High American Society of | 0 | |
| Anesthesiologists class score | | |
| Medical and surgical | 2 | (3.7%) |
| comorbidities | | |
| Elected not to undergo RC | 51 | (94.4%) |
| Preservation of bladder | 28 | (51.9%) |
| Preservation sexual function | 1 | (1.9%) |
| Concern about quality of life | 20 | (37.0%) |
| after cystectomy | | |
| Concern about mortality & | 2 | (3.7%) |
| morbidity risk associated with | | |
| procedure | | |

Key: RC = radical cystectomy
^{a}Stages are mutually exclusive. Urotherial carcinoma in situ includes subjects with CIS only. CIS + pTa includes subjects who have both CIS and pTa. CIS + pT1 includes subjects with CIS and pT1 or CIS, pTa, and pT1.

TABLE 9C
NMIBC Prior Therapies for TAR-200
(Cohort 1); Treated Analysis Set

| | Cohort 1 | |
|---|---|---|
| | TAR-200 | |
| Analysis set: Full | 54 | |
| Total number of Subjects with any Prior | | |
| Therapies for NMIBC | 54 | (100.0%) |
| Prior intravesical BCG therapy | 54 | (100.0%) |
| Prior intravesical therapy (other than BCG) | 4 | (7.4%) |
| Prior NMIBC cancer-related surgery/procedure | 46 | (85.2%) |
| Prior systemic therapy | 0 | |
| Number of Subjects with Adequate | 53 | (98.1%) |
| Prior BCG Therapy | | |
| Number of Induction Phase Doses of Prior | | |
| Intravesical BCG Therapy | | |
| N | 54 | |
| 1 | 0 | |
| 2 | 0 | |
| 3 | 0 | |
| 4 | 0 | |
| >=5 | 54 | (100.0%) |
| Number of Maintenance Phase/Second Induction | | |
| Doses of Prior Intravesical BCG Therapy | | |
| N | 53 | |
| 1 | 0 | |
| 2 | 7 | (13.0%) |
| 3 | 10 | (18.5%) |
| 4 | 1 | (1.9%) |
| >=5 | 35 | (64.8%) |
| Number of Prior Intravesical BCG Doses | | |
| N | 54 | |
| Mean (SD) | 13.9 | (6.26) |

| | |
|---|---|
| Median | 12.0 |
| Range | (7; 42) |

Note:
Each prior therapy is only counted once.
Percentages are calculated with the number of subjects in Full analysis set of each treatment group as the denominator.
Clinical Cutoff Date = 24 Aug. 2023.

The preliminary efficacy assessment was performed on the full analysis set in the TAR-200 monotherapy cohort (n=54) (Table 10A). The overall CR rate (95% CI) based on central review for the full analysis set was 76.7% (95% CI: 57.7%, 90.1%) in TAR-200 monotherapy cohort. The overall CR rate (95% CI) based on investigator assessment for the full analysis set was 80.0% (95% CI: 61.4%, 92.3%) in TAR-200 monotherapy cohort.

The preliminary efficacy assessment was also performed on the evaluable analysis set in the TAR-200 monotherapy cohort (n=28) (Table 10B). The Evaluable Analysis Set is defined as all treated participants who had active disease at baseline and adequate disease assessment post-baseline, or who had progressed, died, been withdrawn from treatment due to recurrence of high-risk disease or progressive disease. The overall CR rate (95% CI) based on central review for the Evaluable Analysis Set was 82.1% (95% CI: 63.1%, 93.9%) in TAR-200 monotherapy cohort. The overall concordance rate between central and investigator assessment was 92.6% (Table 11). Of the 24 CRs by investigator, 22 CRs were confirmed by central assessment.

TABLE 10A
Summary of Best Overall Response by Central Review
for TAR-200 (Cohort 1); Treated Analysis Set

| | Cohort 1 |
|---|---|
| | TAR-200 |
| Analysis set: Full | 54 |
| Evaluable^{a} or Discontinued | |
| Study | 30 |
| Best overall response | |
| Complete response (CR)^{b} | 23 (76.7%) |
| 95% CI^{c} | (57.7%, 90.1%) |
| Non-complete response | |
| (Non-CR)^{d} | 7 (23.3%) |

Note:
Response is based on centrally reviewed urine cytology, local cystoscopy, and central biopsy (if available). CR do not have to be confirmed.
^{a}Evaluable Analysis Set is defined as all treated participants who have active disease at baseline and adequate disease assessment post-baseline, or who have progressed, died, been withdrawn from treatment due to recurrence of high-risk disease or progressive disease.
^{b}A complete response is defined as having a negative cystoscopy and negative (including atypical) centrally read urine cytology, or positive cystoscopy with biopsy-proven benign or low-grade NMIBC and negative (including atypical) centrally read cytology at any time point.
^{c}95% CI is estimated using the exact method based on binomial distribution.
^{d}2 subjects discontinued the study before having a disease evaluation, but are included in the denominator of the evaluation of CR rate.
Clinical Cutoff Date = 24 Aug. 2023.

TABLE 10B
Summary of Best Overall Response by Central Review
for TAR-200 (Cohort 1); Evaluable Analysis Set

| | Cohort 1 |
|---|---|
| | TAR-200 |
| Analysis set: Evaluable^{a} | 28 |
| Best overall response | |
| Complete response (CR)^{b} | 23 (82.1%) |
| 95% CI^{c} | (63.1%, 93.9%) |
| Non-complete response | 5 (17.9%) |
| (Non-CR) | |
| p-value^{d} | <.0001 |

Note:
Response is based on centrally reviewed urine cytology, local cystoscopy, and central biopsy (if available). CR do not have to be confirmed.
^{a}Evaluable Analysis Set is defined as all Safety participants who have active disease at baseline and adequate disease assessment post-baseline, or who have progressed, died, been withdrawn from treatment due to recurrence of high-risk disease or progressive disease.
^{b}A Complete Response is defined as having a negative central cytology and negative central biopsy, if available.
^{c}95% CI is estimated using the exact method based on binomial distribution.
^{d}p-value is from the Z test with normal approximation (comparing to 20%).
Clinical Cutoff Date = 24 Aug. 2023.

TABLE 11
Concordance of Centrally Reviewed and Investigator Assessment
for Response for TAR-200 (Cohort 1); Evaluable Analysis Set

| | Overall Response by Investigator^{a} | | | |
|---|---|---|---|---|
| Overall Response by Central Review^{a} | CR | Non-CR | Total | |
| CR | 22 (81.5%) | 0 | 22 (81.5%) | |
| Non-CR | 2 (7.4%) | 3 (11.1%) | 5 (18.5%) | |
| Total | 24 (88.9%) | 3 (11.1%) | 27 (100.0%) | |
| Overall Concordance^{b} | | | | 25 (92.6%) |

^{a}Includes subjects with central and investigator response available.
^{b}Includes subjects who had CR by both investigator and central review and subjects who had non-CR by both investigator and central review.
Note:
Response for centrally reviewed is based on urine cytology, local cystoscopy, and biopsy (if available).
Percentages are calculated using the number of subjects with central and investigator response available as denominator. Clinical Cutoff Date = 24 Aug. 2023.

The duration of response for individuals treated with TAR-200 in the Evaluable Analysis Set is shown in Table 12 and FIG. 4C. At the clinical cutoff date of Aug. 24, 2023, 21 of 23 responses were ongoing. At 3 months after initial CR, 14 of 23 patients had ongoing response. Notably, 11 patients had a duration of response of at least 6 months (10 of 11 ongoing), and 6 patients had a duration of response of at least 12 months (all ongoing). None of the patients with CR have undergone cystectomy.

TABLE 12
Summary of Duration of Response for TAR-
200 (Cohort 1); Evaluable Analysis Set

| | Cohort 1 |
|---|---|
| | TAR-200 |
| Analysis set: Evaluable | 28 |
| Subjects with Complete Response | 23 |
| Duration of Response (months) | |
| Number of Events(%)^{a} | 2 (8.7%) |
| Number of Censored(%)^{b} | 21 (91.3%) |
| Kaplan-Meier Estimate (months) | |
| 25th percentile (95% CI) | NE (2.79, NE) |
| Median (95% CI) | NE (8.34, NE) |
| 75th percentile (95% CI) | NE (NE, NE) |
| 6-month Event-free (95% CI) | 93.3 (61.3, 99.0) |
| 12-month Event-free (95% CI) | 84.0 (48.7, 95.9) |
| 18-month Event-free (95% CI) | 84.0 (48.7, 95.9) |
| 24-month Event-free (95% CI) | NE (NE, NE) |
| 30-month Event-free (95% CI) | NE (NE, NE) |

Key: CI = confidence interval, NE = not estimable
^{a}Event is defined as recurrence, progression, or death.
^{b}Subjects without an event are censored at last disease assessment.
Clinical Cutoff Date = 24 Aug. 2023.

The preliminary safety assessment includes data on a total of 54 patients with TAR-200 monotherapy (Table 13). Overall, 37 (68.5%) patients in TAR-200 monotherapy experienced at least one treatment-emergent adverse event (TEAE) during the reporting period. Micturition urgency (18.5%), pollakiuria (22.2%), dysuria (20.4%), haematuria (11.1%), and cystitis noninfective (7.4%), were the most commonly reported TEAEs (Table 14). Additionally, 29 patients (53.7%) had at least 1 treatment-related adverse event. One patient (1.9%) had at least one serious treatment-related adverse event. Four patients (7.4%) had grade ≥3 treatment-related adverse events. A low rate of treatment discontinuation due to adverse events was observed (2 patients, 3.7%). No deaths were reported.

Additional analysis of the safety assessment at the 24 Aug. 2023 data cutoff showed that 29 of 54 patients (54%) reported treatment-related adverse events (TRAEs). The most common treatment-related adverse events (TRAEs) (≥5%) were pollakiuria (22%), dysuria (20%), micturition urgency (19%), hematuria (9%) and urinary tract pain (6%); 4 (7%) had grade (Gr)≥3 TRAEs and 1 (2%) had serious TRAE. TRAEs lasted for a median duration of 15 days (interquartile range, 5-64). Only 2 patients (4%) with noninfective cystitis (1 Gr 1; 1 Gr 2) discontinued treatment; both within 3.3 months of starting treatment. No deaths were reported.

TABLE 13
Overall Summary of TAR-200 Treatment-emergent
Adverse Events (Cohort 1); Safety Analysis Set

Cohort 1
TAR-200

| Analysis set: Safety | 54 | |
|---|---|---|
| Subjects with 1 or more: | | |
| TEAEs | 37 | (68.5%) |
| Related TEAEs^{a} | 29 | (53.7%) |
| TAR-200 | 27 | (50.0%) |
| Insertion Procedure | 11 | (20.4%) |
| Removal Procedure | 4 | (7.4%) |
| Urinary Placement Catheter | 8 | (14.8%) |
| Grade >=3 Related TEAEs^{a} | 4 | (7.4%) |
| TAR-200 | 4 | (7.4%) |
| Insertion Procedure | 1 | (1.9%) |
| Removal Procedure | 0 | |
| Urinary Placement Catheter | 1 | (1.9%) |
| Serious TEAEs | 3 | (5.6%) |
| Related Serious TEAEs^{a} | 1 | (1.9%) |
| TAR-200 | 1 | (1.9%) |
| Insertion Procedure | 0 | |
| Removal Procedure | 0 | |
| Urinary Placement Catheter | 0 | |
| TEAEs Leading to Treatment Discontinuation^{b} | 2 | (3.7%) |
| Related TEAEs Leading to Treatment | 2 | (3.7%) |
| Discontinuation^{a,b} | | |
| TAR-200 | 1 | (1.9%) |
| Insertion Procedure | 1 | (1.9%) |
| Removal Procedure | 0 | |
| Urinary Placement Catheter | 0 | |
| TEAEs with Fatal Outcome^{c} | 0 | |
| Related TEAEs with Fatal Outcome^{a,c} | 0 | |
| TAR-200 | 0 | |
| Insertion Procedure | 0 | |
| Removal Procedure | 0 | |
| Urinary Placement Catheter | 0 | |
| COVID-19 associated TEAEs ^{d} | 3 | (5.6%) |
| COVID-19 associated Non-serious TEAEs ^{d} | 3 | (5.6%) |

Key:
AE = adverse event;
TEAE = treatment emergent adverse event
^{a}An AE is categorized as related if the investigator determines that there is a possible, probable, or causal relationship between the AE and TAR-200 or cetrelimab or insertion or removal procedure or UPC.
^{b}Number of subjects who experienced AEs related to TAR-200, insertion procedure, removal procedure, or UPC which led to discontinuation of TAR-200.
^{c}Based on the TEAEs with outcome assessed as Fatal, irrespective of the grade.
^{d} COVID-19 associated AEs are based on events that code to a COVID-19 MedDRA term and events that are identified via the COVID-19 Case of AEs form.
Note:
Subjects are counted only once for any given event, regardless of the number of times they actually experienced the event.
Clinical Cutoff Date = 24 Aug. 2023.

TABLE 14
Overall Safety Profile of TAR-200 Monotherapy

TAR-200 (N = 54)

| Patients with events, n (%) | Any grade | | Grade ≥3 | |
|---|---|---|---|---|
| ≥1 AE | 37 | (68.5) | 9 | (16.7) |
| ≥1 treatment-related AE^{a,b} | 29 | (53.7) | 4 | (7.4) |
| Pollakiuria | 12 | (22.2) | 1 | (1.9) |
| Dysuria | 11 | (20.4) | 0 | |
| Micturition urgency | 10 | (18.5) | 0 | |
| Hematuria | 6 | (11.1) | 0 | |
| Noninfective cystitis | 4 | (7.4) | 0 | |
| Urinary tract pain | 3 | (5.6) | 1 | (1.9) |
| Urinary retention | 2 | (3.7) | 1 | (1.9) |
| Renal impairment | 1 | (1.9) | 1 | (1.9) |
| Urosepsis | 1 | (1.9) | 1 | (1.9) |

^{a}An AE was categorized as related if the investigator determines that there was a possible, probable, or causal relationship between the AE and TAR-200 or insertion or removal procedure or urinary placement catheter.
^{b}Treatment-related AEs by preferred term of any grade reported in ≥5% of patients and all treatment-related AEs by preferred term of grade ≥3 are listed.

### Conclusions:

TAR-200 monotherapy is associated with an unprecedented CR rate, durable responses, and a favorable tolerability profile in patients with BCG-unresponsive HR-NMIBC. The overall CR rate with TAR-200 monotherapy was 76.7% in BCG-unresponsive HR NMIBC. TAR-200 provides sustained and durable responses, with 21 of 23 responses ongoing with a median duration of follow-up in responders of 48 weeks. Eleven patients maintained their CR for six months or more, and six patients remained in CR for 12 months or more. Additionally, the CR rate was concordant between central and investigator assessments. TAR-200 monotherapy was well tolerated. Commonly reported, lower urinary TRAEs were symptomatically managed and resolved after a short duration. TAR-200 has a promising safety and efficacy profile as a local, bladder-sparing treatment in HR-NMIBC unresponsive to BCG.

### Example 4

The following analysis of the study described in Examples 1-3, was performed on updated data from patients in Cohort 1 treated through Jan. 2, 2024 (clinical cut-off date). Results for the TAR-200 monotherapy arm are provided.

Eligible patients aged ≥18 years had histologically confirmed carcinoma in situ (CIS)±papillary disease (high-grade Ta, any T1), ECOG performance status 0-2, after adequate BCG with last dose of BCG≤12 months prior to CIS diagnosis. TAR-200 was dosed every 3 weeks through Week 24, then every 12 weeks until Week 96. Response was assessed by cystoscopy and centrally assessed urine cytology, CT/MRI, and bladder biopsy (at Weeks 24, 48, and as clinically indicated). The primary end point was overall complete response (CR) rate. Secondary end points included duration of response (DOR), overall survival, safety, and tolerability.

At the data cutoff of Jan. 2, 2024, 85 patients (median age, 71 y; range, 40-88; 33% with concurrent papillary disease) received TAR-200 monotherapy. 58 patients were efficacy evaluable.

Baseline characteristics, including information on prior BCG therapy are summarized below (Tables 15A, 15B, and 15C).

TABLE 15A
Summary of Demographics and Baseline Characteristics
for TAR-200; Full Analysis Set

| | Cohort 1 | |
|---|---|---|
| | TAR-200 | |
| Analysis set: Full | 85 | |
| Age, (years) | | |
| N | 85 | |
| Mean (SD) | 69.7 | (9.46) |
| Median | 71.0 | |
| Range | (40; 88) | |
| <65 | 24 | (28.2%) |
| 65-74 | 32 | (37.6%) |
| 75-84 | 25 | (29.4%) |
| >=85 | 4 | (4.7%) |
| Sex | | |
| N | 85 | |
| Female | 17 | (20.0%) |
| Male | 68 | (80.0%) |
| Undifferentiated | 0 | |
| Race^{a} | | |
| N | 85 | |
| American Indian or Alaska Native | 0 | |
| Asian | 8 | (9.4%) |
| Black or African American | 2 | (2.4%) |
| Native Hawaiian or Other Pacific | 0 | |
| Islander | | |
| White | 62 | (72.9%) |
| Not Reported | 12 | (14.1%) |
| Unknown | 1 | (1.2%) |
| Ethnicity | | |
| N | 85 | |
| Hispanic or Latino | 8 | (9.4%) |
| Not Hispanic or Latino | 60 | (70.6%) |
| Not Reported | 14 | (16.5%) |
| Unknown | 3 | (3.5%) |
| Region^{b} | | |
| N | 85 | |
| America | 23 | (27.1%) |
| Asia Pacific | 10 | (11.8%) |
| EMEA | 52 | (61.2%) |
| Nicotine Use History | | |
| N | 85 | |
| Current | 8 | (9.4%) |
| Former | 49 | (57.6%) |
| Never | 28 | (32.9%) |

Note:
N's for each parameter reflect non-missing values.
^{a}If multiple Race categories are indicated, the Race is recorded as “Multiple”.
^{b}America includes Canada, USA; Asia Paicifc includes Australia, Japan, South Korea; EMEA includes Belgium, France, Germany, Greece, Italy, Netherlands, Portugal, Russia, Spain, UK
Clinical Cutoff Date = 2 Jan. 2024.

TABLE 15B
Summary of NMIBC Baseline Disease Characteristics
for TAR-200; Full Analysis Set

Cohort 1
TAR-200

| Analysis set: Full | 85 | |
|---|---|---|
| Baseline ECOG | | |
| N | 85 | |
| 0 | 78 | (91.8%) |
| 1 | 7 | (8.2%) |
| 2 | 0 | |
| Tumor Stage^{a} | | |
| N | 85 | |
| Urothelial carcinoma in situ | 57 | (67.1%) |
| CIS + pTa | 20 | (23.5%) |
| CIS + pT1 | 8 | (9.4%) |
| Papillary Disease (pTa, pT1) | | |
| N | 85 | |
| Present | 28 | (32.9%) |
| Absent | 57 | (67.1%) |
| Time from most recent BCG dose to | | |
| diagnosis of BCG unresponsive disease (months) | | |
| N | 83 | |
| Mean (SD) | 4.5 | (3.67) |
| Median | 3.4 | |
| Range | (0; 22) | |
| Reason for not receiving RC | | |
| N | 85 | |
| Ineligible | 3 | (3.5%) |
| Age | 1 | (1.2%) |
| High American Society of | 0 | |
| Anesthesiologists class score | | |
| Medical and surgical comorbidities | 2 | (2.4%) |
| Elected not to undergo RC | 82 | (96.5%) |
| Preservation of bladder | 50 | (58.8%) |
| Preservation sexual function | 1 | (1.2%) |
| Concern about quality of life after | 29 | (34.1%) |
| cystectomy | | |
| Concern about mortality & | 2 | (2.4%) |
| morbidity risk associated with | | |
| procedure | | |

| | |
|---|---|
| Missing | 0 |

Key: RC = radical cystectomy
^{a}Stages are mutually exclusive. Urothelial carcinoma in situ includes subjects with CIS only. CIS + pTa includes subjects who have both CIS and pTa. CIS + pT1 includes subjects with CIS and pT1 or CIS, pTa, and pT1.
Clinical Cutoff Date = 2 Jan. 2024.

TABLE 15C
NMIBC Prior Therapies for TAR-200; Full Analysis Set

Cohort 1
TAR-200

| Analysis set: Full | 85 | |
|---|---|---|
| Total number of Subjects with any Prior | 85 | (100.0%) |
| Therapies for NMIBC | | |
| Prior intravesical BCG therapy | 84 | (98.8%) |
| Prior intravesical therapy (other than | 9 | (10.6%) |
| BCG) | | |
| Prior NMIBC cancer-related | 73 | (85.9%) |
| surgery/procedure | | |
| Prior systemic therapy | 1 | (1.2%) |
| Number of Subjects with Adequate Prior | 81 | (96.4%) |
| BCG Therapy | | |
| Number of Induction Phase Doses of | | |
| Prior Intravesical BCG Therapy | | |
| N | 84 | |
| 1 | 0 | |
| 2 | 0 | |
| 3 | 0 | |
| 4 | 0 | |
| >=5 | 84 | (98.8%) |
| Number of Maintenance Phase/Second | | |
| Induction Doses of Prior Intravesical | | |
| BCG Therapy | | |
| N | 81 | |
| 1 | 0 | |
| 2 | 9 | (10.6%) |
| 3 | 15 | (17.6%) |
| 4 | 2 | (2.4%) |
| >=5 | 55 | (64.7%) |
| Number of Prior Intravesical BCG Doses | | |
| N | 84 | |
| Mean (SD) | 13.8 | (5.76) |

| Median | 12.0 |
|---|---|
| Range | (7; 42) |

Note:
Each prior therapy is only counted once.
Percentages are calculated with the number of subjects in Full analysis set of each treatment group as the denominator.
Clinical Cutoff Date = 2 Jan. 2024.

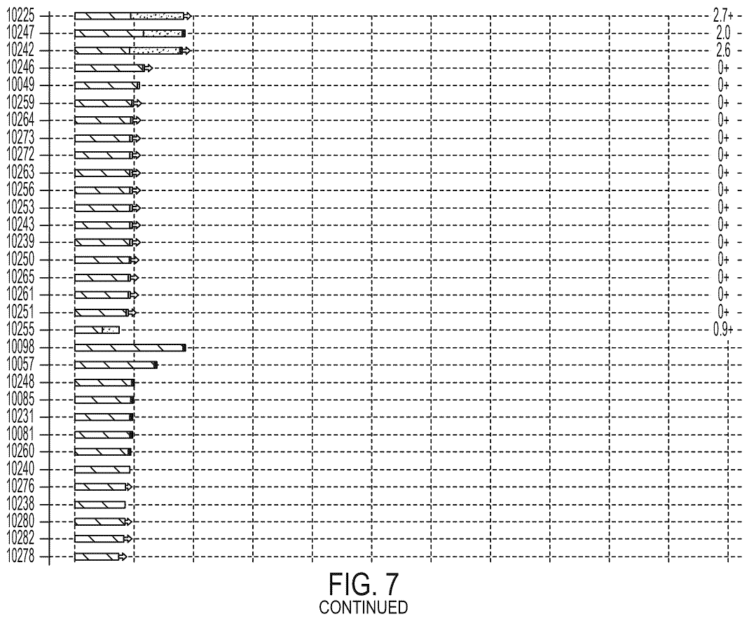

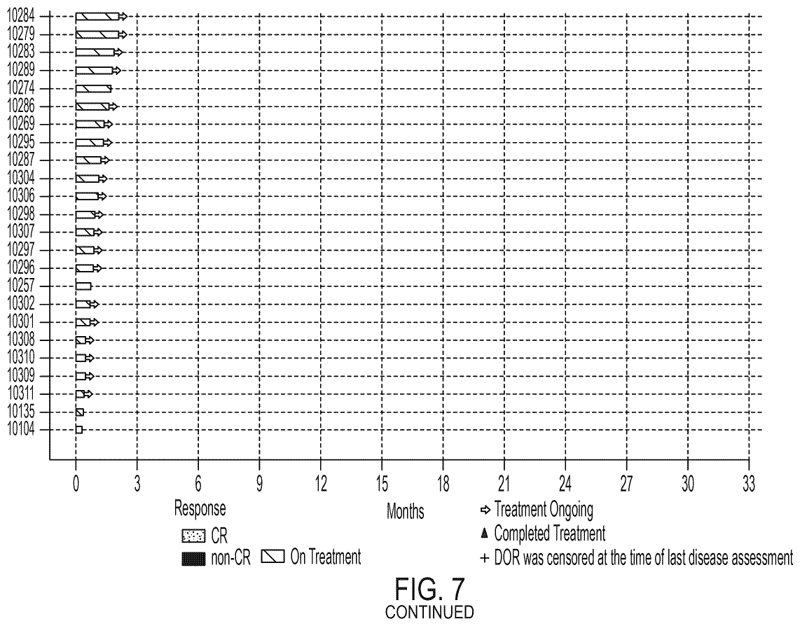

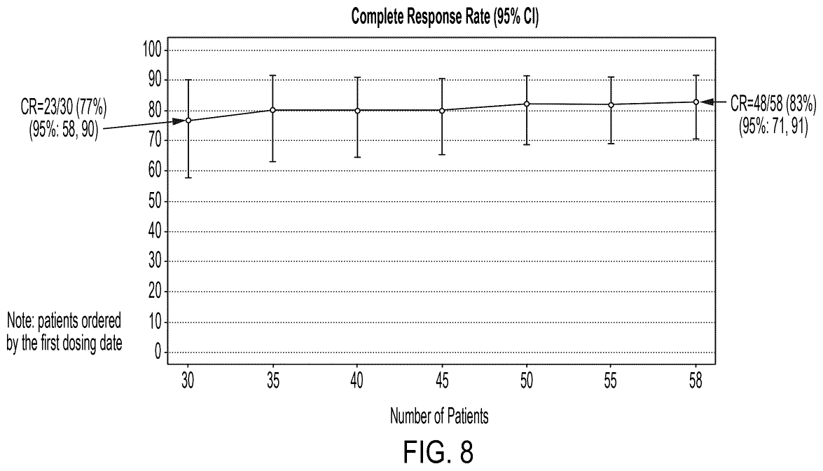

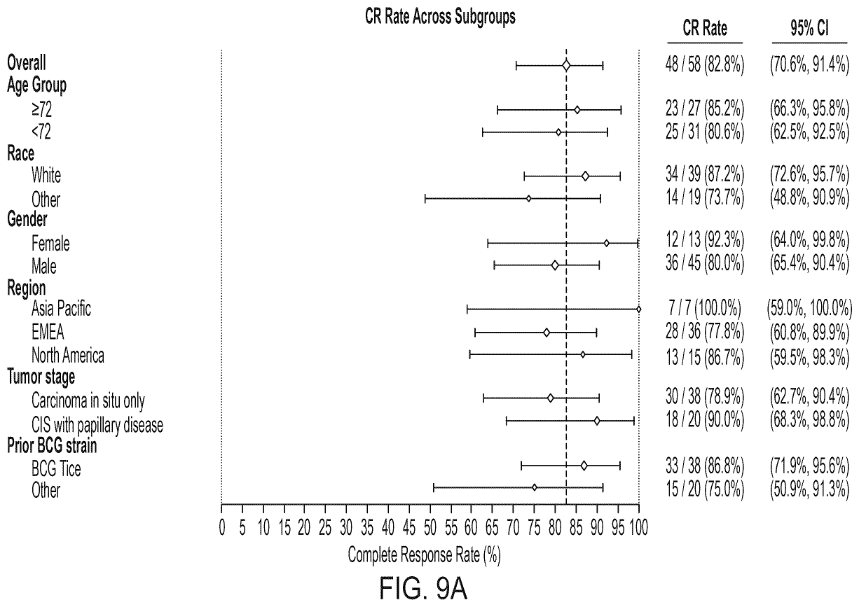

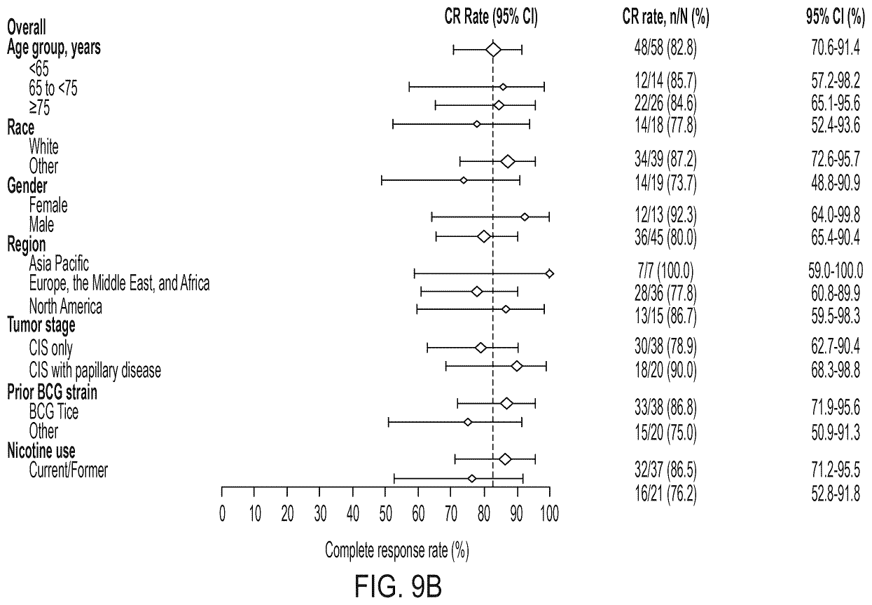

Centrally confirmed CR by urine cytology and/or biopsy was achieved in 48 of 58 patients (83%; 95% CI, 71-91) (Table 16A, FIG. 8). As shown in FIGS. 9A and 9B, the complete response rate was consistent across patient subgroups including age, race, gender, region, tumor stage, and prior BCG strain. The overall response by central or investigator review was also calculated for the evaluable analysis set (Tables 16B, 16C). Additionally, 47 out of 48 patients achieved a CR at the 12 weeks disease assessment. The CR rate by investigator assessment (86%; 95% CI, 75-94) correlated strongly with central results (Table 17).

TABLE 16A
Summary of Best Overall Response by Central
Review for TAR-200; Full Analysis Set

| | Cohort 1 |
|---|---|
| | TAR-200 |
| Analysis set: Full | 85 |
| Evaluable^{a} or Discontinued Study | 58 |
| Best overall response | |
| Complete response (CR)^{b} | 48 (82.8%) |
| 95% CI^{c} | (70.6%, 91.4%) |
| Non-complete response (Non-CR)^{d} | 10 (17.2%) |

Note:
Response is based on centrally reviewed urine cytology, local cystoscopy, and central biopsy (if available). CR do not have to be confirmed.
^{a}Evaluable Analysis Set is defined as all treated participants who have active disease at baseline and adequate disease assessment post-baseline, or who have progressed, died, been withdrawn from treatment due to recurrence of high-risk disease or progressive disease.
^{b}A complete response is defined as having a negative cystoscopy and negative (including atypical) centrally read urine cytology, or positive cystoscopy with biopsy-proven benign or low-grade NMIBC and negative (including atypical) centrally read cytology at any time point.
^{c}95% CI is estimated using the exact method based on binomial distribution.
^{d}2 subjects discontinued the study before having a disease evaluation, but are included in the denominator of the evaluation of CR rate.
Clinical Cutoff Date = 2 Jan. 2024.

TABLE 16B
Summary of Best Overall Response by Central
Review for TAR-200; Evaluable Analysis Set

| | Cohort 1 | |
|---|---|---|
| | TAR-200 | |
| Analysis set: Evaluable^{a} | 55 | |
| Best overall response | | |
| Complete response (CR)^{b} | 48 | (87.3%) |
| 95% CI^{c} | (75.5%, 94.7%) | |
| Non-complete response (Non-CR) | 7 | (12.7%) |

| | |
|---|---|
| p-value^{d} | <.0001 |

Note:
Response is based on centrally reviewed urine cytology, local cystoscopy, and central biopsy (if available). CR do not have to be confirmed.
^{a}Evaluable Analysis Set is defined as all Safety participants who have active disease at baseline and adequate disease assessment post-baseline, or who have progressed, died, been withdrawn from treatment due to recurrence of high-risk disease or progressive disease.
^{b}A Complete Response is defined as having a negative central cytology and negative central biopsy, if available.
^{c}95% CI is estimated using the exact method based on binomial distribution.
^{d}p-value is from the Z test with normal approximation (comparing to 20%).
Clinical Cutoff Date = 2 Jan. 2024.

TABLE 16C
Summary of Best Overall Response by Central and Investigator Review
for TAR-200; Evaluable Analysis set or Study Discontinued

| | Cohort 1 | | |
|---|---|---|---|
| | TAR-200 | | |
| | Central | Investigator | |
| Evaluable^{a} or Discontinued Study | 58 | 58 | |
| Best overall response | | | |
| Complete response (CR)^{b} | 48 (82.8%) | 50 | (86.2%) |
| 95% CI^{c} | (70.6%, 91.4%) | (74.6%, 93.9%) | |
| Non-complete response (Non- | 10 (17.2%) | 8 | (13.8%) |
| CR)^{d} | | | |

Note:
Response is based on centrally reviewed urine cytology, local cystoscopy, and central biopsy (if available). CR do not have to be subsequently confirmed.
^{a}Evaluable Analysis Set is defined as all treated participants who have active disease at baseline and adequate disease assessment post-baseline, or who have progressed, died, been withdrawn from treatment due to recurrence of high-risk disease or progressive disease.
^{b}A complete response is defined as having a negative cystoscopy and negative (including atypical) centrally read urine cytology, or positive cystoscopy with biopsy-proven benign or low-grade NMIBC and negative (including atypical) centrally read cytology at any time point.
^{c}95% CI is estimated using the exact method based on binomial distribution.
^{d}Non-CR includes participants who discontinued treatment without any post-baseline disease assessment. Two participants who discontinued prior to the first disease assessment were counted as non-CR per central or investigator review.
Clinical Cutoff Date = 2 Jan. 2024.

TABLE 17
Concordance of Centrally Reviewed and Investigator Assessment
for Response for TAR-200; Evaluable Analysis Set

| | Overall Response by Investigator^{a} | | | | | |
|---|---|---|---|---|---|---|
| | CR | | Non-CR | Total | | |
| Overall Response by Central Review^{a} | | | | | | |
| CR | 48 | (87.3%) | 0 | 48 | (87.3%) | |
| Non-CR | 1 | (1.8%) | 6 (10.9%) | 7 | (12.7%) | |
| Total | 49 | (89.1%) | 6 (10.9%) | 55 | (100.0%) | |
| Overall Concordance^{b} | | | | | | 54 (98.2%) |

^{a}Includes subjects with central and investigator response available.
^{b}Includes subjects who had CR by both investigator and central review and subjects who had non-CR by both investigator and central review.
Note:
Response for centrally reviewed is based on urine cytology, local cystoscopy, and biopsy (if available). Percentages are calculated using the number of subjects with central and investigator response available as denominator. Clinical Cutoff Date = 2 Jan. 2024.

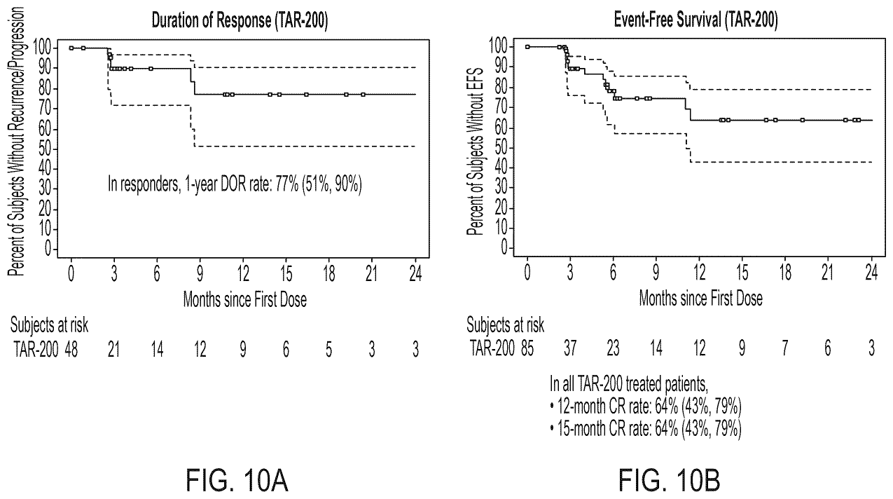

The estimated 1-year DOR rate is 77% (95% CI 50-89), with median follow-up in responders of 30 weeks (range, 14-140); 41 of 48 responders (85%) remain in CR at data cutoff (FIG. 10A) (Table 18). In contrast to the 1-year DOR rate, which only measures responses in patients who have responded to treatment, 12-month and 15-month CR rates were calculated from all TAR-200 treated patients. The 12-month CR rate was 64% (95% CI 43%-79%) and the 15-month CR rate was 64% (95% CI 43%-79%) (FIG. 10B).

TABLE 18
Summary of Duration of Response for TAR-200;
Evaluable Analysis set or Study Discontinued

| | Cohort 1 | |
|---|---|---|
| | TAR-200 | |
| Analysis set: Evaluable or Discontinued Study | 58 | |
| Subjects with Complete Response | 48 | |
| Duration of Response (months) | | |
| Number of Events (%)^{a} | 7 | (14.6%) |
| Number of Censored (%)^{b} | 41 | (85.4%) |
| Kaplan-Meier Estimate (months) | | |
| 25th percentile (95% CI) | 8.61 | (2.76, NE) |
| Median (95% CI) | 25.79 | (8.61, NE) |
| 75th percentile (95% CI) | NE | (25.79, NE) |
| 6-month Event-free (95% CI) | 87.0 | (69.0, 94.9) |
| 12-month Event-free (95% CI) | 74.6 | (49.8, 88.4) |
| 18-month Event-free (95% CI) | 74.6 | (49.8, 88.4) |
| 24-month Event-free (95% CI) | 74.6 | (49.8, 88.4) |
| 30-month Event-free (95% CI) | NE | (NE, NE) |

Key:
CI = confidence interval,
NE = not estimable
Note:
Evaluable Analysis Set is defined as all treated participants who have active disease at baseline and adequate disease assessment post-baseline, or who have progressed, died, been withdrawn from treatment due to recurrence of high-risk disease or progressive disease.
^{a}Event is defined as recurrence, progression, or death.
^{b}Subjects without an event are censored at last disease assessment.
Clinical Cutoff Date = 2 Jan. 2024.

61 patients (72%) had treatment-related adverse events (TRAEs); most common (≥10%) were pollakiuria (35%), dysuria (29%), micturition urgency (15%), and urinary tract infection (15%) (Tables 19A, 19B). 7 patients (8%) had grade ≥3 TRAEs; 4 (5%) had serious TRAEs; 4 (5%) had TRAEs leading to discontinuation. No treatment-related deaths were reported.

TABLE 19A
Overall Summary of TAR-200 Treatment-emergent
Adverse Events; Safety Analysis Set

| | Cohort 1 | |
|---|---|---|
| | TAR-200 | |
| Analysis set: Safety | 85 | |
| Subjects with 1 or more: | | |
| TEAEs | 73 | (85.9%) |
| Related TEAEs | 61 | (71.8%) |
| TAR-200 | 54 | (63.5%) |
| Insertion Procedure | 28 | (32.9%) |
| Removal Procedure | 14 | (16.5%) |
| Urinary Placement Catheter | 17 | (20.0%) |
| Grade >=3 TEAEs | 19 | (22.4%) |
| Grade >=3 Related TEAE | 7 | (8.2%) |
| TAR-200 | 7 | (8.2%) |
| Insertion Procedure | 0 | |
| Removal Procedure | 0 | |
| Urinary Placement Catheter | 1 | (1.2%) |
| Serious TEAEs | 12 | (14.1%) |
| Related Serious TEAEs | 4 | (4.7%) |
| TAR-200 | 3 | (3.5%) |
| Insertion Procedure | 1 | (1.2%) |
| Removal Procedure | 0 | |
| Urinary Placement Catheter | 0 | |
| TEAEs Leading to Treatment | 5 | (5.9%) |
| Discontinuation^{a} | | |
| Related TEAEs Leading to Treatment | 4 | (4.7%) |
| Discontinuation^{a} | | |
| TAR-200 | 2 | (2.4%) |
| Insertion Procedure | 2 | (2.4%) |
| Removal Procedure | 1 | (1.2%) |

| | |
|---|---|
| Urinary Placement Catheter | 0 |
| TEAEs with Fatal Outcome^{b} | 0 |
| Related TEAEs with Fatal Outcome^{b} | 0 |
| TAR-200 | 0 |
| Insertion Procedure | 0 |
| Removal Procedure | 0 |
| Urinary Placement Catheter | 0 |

Key:
AE = adverse event;
TEAE = treatment emergent adverse event
Note:
An AE is categorized as related for insertion or removal procedure or UPC if the investigator determines that there is a possible, probable, or causal relationship between the AE and insertion or removal procedure or UPC.
Note:
Subjects are counted only once for any given event, regardless of the number of times they actually experienced the event.
^{a}Number of subjects who experienced AEs related to TAR-200, insertion procedure, removal procedure, or UPC which led to discontinuation of TAR-200.
^{b}Based on the TEAEs with outcome assessed as Fatal, irrespective of the grade.
Clinical Cutoff Date = 2 Jan. 2024.

TABLE 19B
Number of Subjects in TAR-200 Cohort with Related
Treatment-emergent Adverse Events by System Organ
Class and Preferred Term; Safety Analysis Set

| | Cohort 1 | |
|---|---|---|
| | TAR-200 | |
| Analysis set: Safety | 85 | |
| Subjects with 1 or more Related | 61 | (71.8%) |
| TEAEs | | |
| System organ class | | |
| Preferred term | | |
| Renal and urinary disorders | 56 | (65.9%) |
| Pollakiuria | 30 | (35.3%) |
| Dysuria | 25 | (29.4%) |
| Micturition urgency | 13 | (15.3%) |
| Haematuria | 8 | (9.4%) |
| Cystitis noninfective | 7 | (8.2%) |
| Urinary tract pain | 6 | (7.1%) |
| Bladder pain | 3 | (3.5%) |
| Bladder spasm | 3 | (3.5%) |
| Lower urinary tract symptoms | 3 | (3.5%) |
| Urinary incontinence | 3 | (3.5%) |
| Nocturia | 2 | (2.4%) |
| Urethral pain | 2 | (2.4%) |
| Urinary retention | 2 | (2.4%) |
| Bladder discomfort | 1 | (1.2%) |
| Bladder irritation | 1 | (1.2%) |
| Haemorrhage urinary tract | 1 | (1.2%) |
| Polyuria | 1 | (1.2%) |
| Renal impairment | 1 | (1.2%) |
| Infections and infestations | 16 | (18.8%) |
| Urinary tract infection | 13 | (15.3%) |
| Cystitis | 2 | (2.4%) |
| Cystitis pseudomonal | 1 | (1.2%) |
| Urethritis | 1 | (1.2%) |
| Urinary tract infection bacterial | 1 | (1.2%) |
| Urosepsis | 1 | (1.2%) |
| Gastrointestinal disorders | 7 | (8.2%) |
| Constipation | 3 | (3.5%) |
| Abdominal pain | 2 | (2.4%) |
| Abdominal pain lower | 1 | (1.2%) |
| Gastrooesophageal reflux disease | 1 | (1.2%) |
| Vomiting | 1 | (1.2%) |
| Reproductive system and breast | 5 | (5.9%) |
| disorders | | |
| Pelvic pain | 2 | (2.4%) |
| Penile pain | 2 | (2.4%) |
| Perineal pain | 1 | (1.2%) |
| Retrograde ejaculation | 1 | (1.2%) |
| Vulvovaginal pain | 1 | (1.2%) |
| General disorders and administration | 3 | (3.5%) |
| site conditions | | |
| Asthenia | 2 | (2.4%) |
| Fatigue | 2 | (2.4%) |
| Injury, poisoning and procedural | 2 | (2.4%) |
| complications | | |
| Procedural complication | 1 | (1.2%) |
| Urethral injury | 1 | (1.2%) |
| Musculoskeletal and connective tissue | 2 | (2.4%) |
| disorders | | |
| Flank pain | 1 | (1.2%) |
| Myalgia | 1 | (1.2%) |
| Investigations | 1 | (1.2%) |
| Nitrite urine present | 1 | (1.2%) |

Key:
AE = adverse event;
TEAE = treatment emergent adverse event
Note:
An AE is categorized as related if the investigator determines that there is a relationship between the AE and study drug/procedure. Subjects are counted only once for any given event, regardless of the number of times they actually experienced the event. Two subjects were assigned to receive TAR-200 and cetrelimab (cohort 1), but only received cetrelimab. Adverse events are coded using MedDRA Version 26.1.
Clinical Cutoff Date = 2 Jan. 2024.

### Conclusions:

TAR-200 monotherapy is associated with a clinically meaningful, high centrally confirmed CR rate, durable responses, and a favorable benefit-risk profile in patients with BCG-unresponsive CIS.

### Example 5

The following analysis of the study described in Examples 1-4, was performed on updated data from patients in Cohort 1 treated through Feb. 25, 2024 (clinical cut-off date). The median duration of study was 34.3 weeks (Table 20). Results for the TAR-200 monotherapy arm are provided.

TABLE 20
Summary of Study Duration for TAR-200; Evaluable Analysis set

| | Cohort 1 | |
|---|---|---|
| | TAR-200 | |
| Analysis set: Evaluable | 66 | |
| Subjects with Complete Response | 56 | |
| Duration on study (Weeks)^{a} | | |
| N | 56 | |
| Mean (SD) | 47.8 | (34.97) |
| Median | 34.3 | |
| Range | (13; 148) | |
| >0 | 56 | (100.0%) |
| >=12 | 56 | (100.0%) |
| >=24 | 43 | (76.8%) |
| >=36 | 25 | (44.6%) |
| >=48 | 16 | (28.6%) |
| >=60 | 14 | (25.0%) |
| >=72 | 11 | (19.6%) |
| >=84 | 8 | (14.3%) |
| >=96 | 7 | (12.5%) |

^{a}Duration is calculated from treatment start date to study completion date, withdrawal date, or clinical cut-off date, whichever is earliest.
Clinical Cutoff Date = 25 Feb. 2024.

A total of 66 participants were evaluable, with 56 participants achieving CR, representing an overall centrally assessed CR rate of 81.2% (95% CI: 69.9, 89.6). The CR rate by investigator assessment (83%; 95% CI, 72-91) correlated strongly with central results (Table 21). Furthermore, the centrally estimated CR rate was 84.1% (95% CI: 67.5; 92.6), 67.3% (95% CI: 43.8; 82.6) and 67.3% (95% CI: 43.8, 82.6) at 6, 12 and 18 months, respectively, based on Kaplan-Meier estimate of event-free survival (ie, defined as time from first dose to the recurrence/progression or death, which occurs earlier).

TABLE 21
Summary of Best Overall Response by Central and Investigator Review
for TAR-200; Evaluable Analysis set or Study Discontinued

| | Cohort 1 | |
|---|---|---|
| | TAR-200 | |
| | Central | Investigator |
| Evaluable^{a} or Discontinued Study | 69 | 69 |
| Best overall response | | |
| Complete response (CR)^{b} | 56 (81.2%) | 57 (82.6%) |
| 95% CI^{c} | (69.9%, 89.6%) | (71.6%, 90.7%) |
| Non-complete response (Non-CR)^{d} | 13 (18.8%) | 12 (17.4%) |

Note:
Response is based on centrally reviewed urine cytology, local cystoscopy, and central biopsy (if available). CR do not have to be confirmed.
^{a}Evaluable Analysis Set is defined as all treated participants who have active disease at baseline and adequate disease assessment post-baseline, or who have progressed, died, been withdrawn from treatment due to recurrence of high-risk disease or progressive disease.
^{b}A complete response is defined as having a negative cystoscopy and negative (including atypical) centrally read urine cytology, or positive cystoscopy with biopsy-proven benign or low-grade NMIBC and negative (including atypical) centrally read cytology at any time point.
^{c}95% CI is estimated using the exact method based on binomial distribution.
^{d}Non-CR includes participants who discontinued treatment without any post-baseline disease assessment. Two participants who discontinued prior to the first disease assessment were counted as non-CR per central or investigator review.
Clinical Cutoff Date = 25 Feb. 2024.

The duration of response for individuals treated with TAR-200 in the Evaluable Analysis Set is shown in Table 22. The estimated median DOR was 25.8 weeks (95% CI: 8.61, NE) after a median follow up of 34.3 weeks (range: 13 to 148 weeks) among 56 responding participants.

TABLE 22
Summary of Duration of Response for TAR-200;
Evaluable Analysis set or Study Discontinued

| | Cohort 1 | |
|---|---|---|
| | TAR-200 | |
| Analysis set: Evaluable or Discontinued Study | 69 | |
| Subjects with Complete Response | 56 | |
| Duration of Response (months) | | |
| Number of Events (%)^{a} | 10 | (17.9%) |
| Recurrence/Progression^{b} | 9 | (16.1%) |
| Death | 1 | (1.8%) |
| Number of Censored (%)^{c} | 46 | (82.1%) |
| Kaplan-Meier Estimate (months) | | |
| 25th percentile (95% CI) | 8.34 | (2.79, NE) |
| Median (95% CI) | 25.79 | (8.61, NE) |
| 75th percentile (95% CI) | NE | (25.79, NE) |
| 6-month Event-free (95% CI) | 84.1 | (67.5, 92.6) |
| 12-month Event-free (95% CI) | 67.3 | (43.8, 82.6) |
| 18-month Event-free (95% CI) | 67.3 | (43.8, 82.6) |
| 24-month Event-free (95% CI) | 67.3 | (43.8, 82.6) |
| 30-month Event-free (95% CI) | NE | (NE, NE) |

Key:
CI = confidence interval,
NE = not estimable
Note:
Evaluable Analysis Set is defined as all treated participants who have active disease at baseline and adequate disease assessment post-baseline, or who have progressed, died, been withdrawn from treatment due to recurrence of high-risk disease or progressive disease.
^{a}Event is defined as recurrence, progression, or death.
^{b}Subjects without an event are censored at last disease assessment.
Clinical Cutoff Date = 25 Feb. 2024.

### Pharmacokinetics

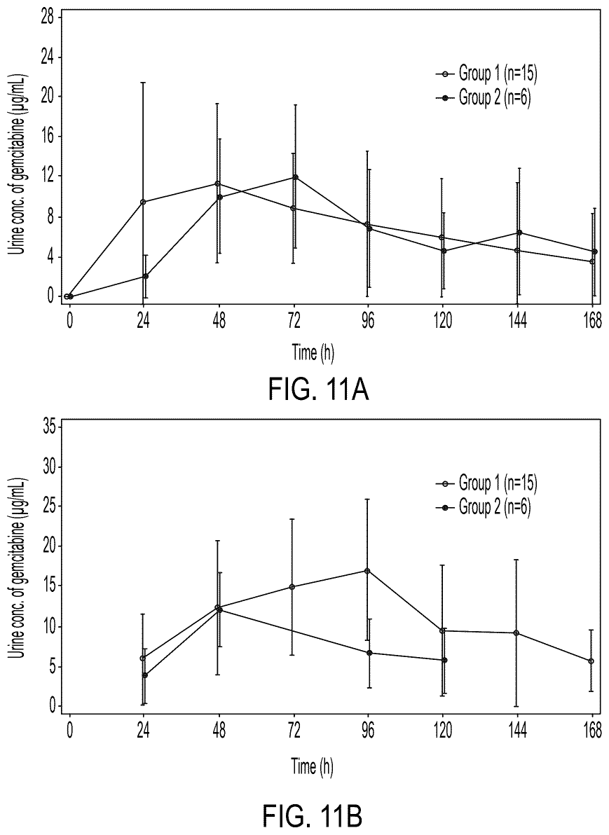

After administration of TAR-200 via the intravesical system, maximum gemcitabine concentration in urine was reached at a median Tₘₐₓ of 2 to 4 days. Mean gemcitabine concentration in urine exceeded the tumoricidal concentrations of 3 μg/mL (FIG. 11A and FIG. 11B). Mean cumulative excreted gemcitabine in urine expressed as percentage of the administered dose from 0-168 hours ranged between 36.2-53.8%. Meanwhile, maximum dFdU concentration in urine was reached at median Tₘₐₓ between 3 and 6 days. Mean cumulative excreted dFdU amount in urine expressed as percentage of the administered dose from 0-168 hours ranged between 11.1-23.3%. The total excreted gemcitabine and dFdU was further converted to Gemcitabine Related Components (GRC) in mg-equivalents gemcitabine (mg-Eq. gemcitabine) and are calculated as follows:

$$GRC ( mg - Eq . gemcitabine ) = Gemcitabine in mg + dFdU in mg * ( 263.2 / 264.18 ) .$$

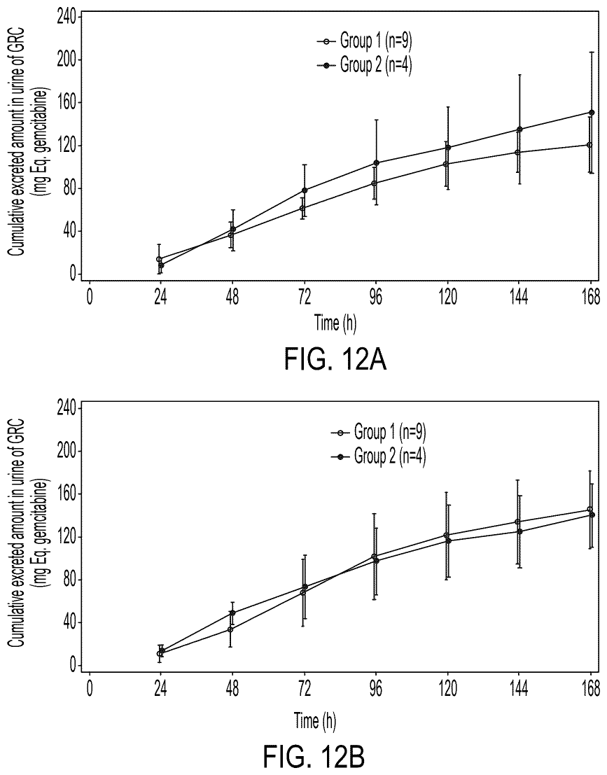

Mean cumulative excreted GRC amount in urine expressed as percentage of the administered dose from 0-168 hours was approximately 60% (FIG. 12A and FIG. 12B).

Plasma gemcitabine concentrations of all available samples were below the limit of quantitation [LOQ 0.1 μg/mL] which confirms that there is no systemic exposure of gemcitabine. Plasma dFdU concentrations of all except 2 samples were below the limit of quantitation [LOQ 0.1 μg/mL] which confirms that there is negligible systemic exposure of dFdU (≤0.397 μg/mL).

### Safety

Overall TEAEs regardless of relationship were reported in 161 participants (89.0%). Of these, TEAEs reported in 106 participants (69.3%) were considered related to TAR-200. Related TEAEs reported in ≥5.0% of participants were pollakiuria (31.4%), dysuria (26.1%), pollakiuria (%), hematuria (12.4%), micturition urgency (14.4%) urinary tract infection (9.2%), cystitis noninfective (8.5%), bladder pain (7.8%), cystitis infective (5.9%) and bladder spasm (5.2%).

Grade ≥3 TEAEs, regardless of relationship, were reported in 56 participants (30.9%). Of these, TEAEs reported in 28 participants (15.5%) were considered related to study treatment or procedure. There were no related Grade ≥3 TEAEs, by SOC and PT, reported in ≥2% of participants.

SAEs were reported in 36 participants (19.9%) and were considered related to study treatment or procedure in 13 participants (7.2%). Regardless of relationship, there were no SAEs by SOC and PT reported in ≥2% participants.

A total of 38 participants (21.0%) discontinued treatment due to TEAEs, with TEAEs considered related to study treatment or procedure in 35 participants (19.3%). Regardless of relationship, TEAEs reported in ≥2% of participants were bladder pain (6.83.9%), pollakiuria (2.8%) and cystitis noninfective (2.2%).

TEAEs leading to death were reported in 1 participant, this was not considered related to study treatment or procedure.

1-89. (canceled)

90. A method of bladder sparing in an individual having non-muscle invasive bladder cancer (NMIBC) that is unresponsive to intravesical Bacillus Calmette-Guerin (BCG) therapy comprising
administering to the bladder of the individual an intravesical drug delivery system comprising about 225 mg of gemcitabine about every three weeks (Q3W) for at least about 24 weeks, followed by
administering to the bladder of the individual an intravesical drug delivery system comprising about 225 mg of gemcitabine about every twelve weeks (Q12W) for at least about 18 months,
wherein the intravesical drug delivery system is removed about 3 weeks after each administration,
wherein the individual does not receive a radical cystectomy,
wherein the concentration of gemcitabine in the urine of the individual is between 4 μg/mL and 20 μg/mL during each delivery period, and
wherein the gemcitabine is administered as a monotherapy.

91. A method of treating non-muscle invasive bladder cancer (NMIBC) that is unresponsive to intravesical Bacillus Calmette-Guerin (BCG) therapy in an individual comprising
administering to the bladder of the individual an intravesical drug delivery system comprising about 225 mg of gemcitabine about every three weeks (Q3W) for at least about 24 weeks, followed by
administering to the bladder of the individual an intravesical drug delivery system comprising about 225 mg of gemcitabine about every twelve weeks (Q12W) for at least about 18 months,
wherein the intravesical drug delivery system is removed about 3 weeks after each administration, and
wherein the complete response rate in a patient population receiving such therapy is between about 71% to about 91%.

92. A method of inducing a complete response (CR) rate of at least 50% in a patient population with non-muscle invasive bladder cancer (NMIBC) that is unresponsive to intravesical Bacillus Calmette-Guerin (BCG) therapy, the method comprising performing the following steps on individuals of the patient population:
i) inserting an intravesical drug delivery system comprising about 225 mg gemcitabine into the bladder of the individual; and
ii) removing the intravesical drug delivery system about three weeks later, wherein i) and ii) are repeated for at least about 24 weeks;
i′) inserting an intravesical drug delivery system comprising about 225 mg gemcitabine into the bladder of an individual with NMIBC; and
ii′) removing the intravesical drug delivery system about three weeks later, wherein i′) and ii′) are repeated for at least about 18 months,
wherein the concentration of gemcitabine in the urine of the individual is between 4 μg/mL and 20 μg/mL during each delivery period, and
wherein the gemcitabine is administered as a monotherapy.

93. The method of claim 92, wherein each intravesical drug delivery system comprises an intravesical device that comprises a housing which contains and controllably releases the gemcitabine and is elastically deformable between a retention shape configured to retain the intravesical device in the bladder of the individual and a deployment shape for passage of the intravesical device through the individual's urethra.

94. The method of claim 92, wherein gemcitabine is released into the bladder during a delivery period,
wherein the delivery period is less than an administration period during which the intravesical drug delivery system is maintained in the bladder, and
wherein each delivery period is about one week to about three weeks.

95. The method of claim 92, wherein the complete response rate in the patient population is between about 71% to about 91%.

96. The method of claim 92, wherein such treatment results in an increased complete response rate compared to the complete response rate achieved by valrubicin.

97. The method of claim 92, wherein a complete response is determined on the basis of a negative cytology analysis and/or on the basis of a negative biopsy.

98. The method of claim 92, wherein the individual is administered gemcitabine within 12 months of completion of a last dose of BCG therapy, and
wherein the individual has received:
i) a minimum of five of six full doses of an induction course of BCG; and
ii) two or three doses of a maintenance course, or two of six doses of a second induction course of BCG therapy.

99. The method of claim 92, wherein the individual has carcinoma in situ (CIS) and/or wherein the patient population comprises one or more individuals with CIS.

100. The method of claim 92, wherein the individual has a histologically confirmed diagnosis of persistent or recurrent CIS and/or wherein the patient population comprises one or more individuals with a histologically confirmed diagnosis of persistent or recurrent CIS.

101. The method of claim 92, wherein the individual has:
Ta stage bladder cancer, and/or wherein the patient population comprises one or more individuals with Ta stage bladder cancer;
high grade Ta stage bladder cancer and/or wherein the patient population comprises one or more individuals with high grade Ta stage bladder cancer;
T1 stage bladder cancer and/or wherein the patient population comprises one or more individuals with T1 stage bladder cancer; or
high grade T1 stage bladder cancer and/or wherein the patient population comprises one or more individuals with high grade T1 stage bladder cancer.

102. The method of claim 92, wherein the individual has papillary bladder cancer and/or wherein the patient population comprises one or more individuals with papillary bladder cancer.

103. The method of claim 92, wherein the individual is ineligible or elected not to undergo radical cystectomy.

104. The method of claim 92, comprising performing a transurethral resection of bladder tumor (TURBT) if the individual has papillary bladder cancer.

105. The method of claim 92, wherein the individual has carcinoma in situ (CIS) following TURBT.

106. The method of claim 92, wherein:
the individual is renally impaired and/or hepatically impaired;
the individual is elderly and/or frail; and/or
the individual is at least 65 years old.

107. The method of claim 92, wherein:
treatment with gemcitabine causes tumor ablation;
following treatment with gemcitabine, the individual does not receive a transurethral resection of bladder tumor (TURBT);
the individual does not progress to muscle-invasive bladder cancer; and/or
the quality-of-life score of the individual is maintained or increases following treatment with gemcitabine.

108. The method of claim 92, comprising:
a dosing schedule of at least 99 weeks comprising
i) inserting a first intravesical drug delivery system into the bladder of the individual in week 0, and removing the first intravesical drug delivery system in week 3;
ii) inserting a second intravesical drug delivery system into the bladder of the individual in week 3 after removing the first intravesical drug delivery system, and removing the second intravesical drug delivery system in week 6;
iii) inserting a third intravesical drug delivery system into the bladder of the individual in week 6 after removing the second intravesical drug delivery system, and removing the third intravesical drug delivery system in week 9;
iv) inserting a fourth intravesical drug delivery system into the bladder of the individual in week 9 after removing the third intravesical drug delivery system, and removing the fourth intravesical drug delivery system in week 12;
v) inserting a fifth intravesical drug delivery system into the bladder of the individual in week 12 after removing the fourth intravesical drug delivery system, and removing the fifth intravesical drug delivery system in week 15;
vi) inserting a sixth intravesical drug delivery system into the bladder of the individual in week 15 after removing the fifth intravesical drug delivery system, and removing the sixth intravesical drug delivery system in week 18;
vii) inserting a seventh intravesical drug delivery system into the bladder of the individual in week 18 after removing the sixth intravesical drug delivery system, and removing the seventh intravesical drug delivery system in week 21;
viii) inserting an eighth intravesical drug delivery system into the bladder of the individual in week 21 after removing the seventh intravesical drug delivery system, and removing the eighth intravesical drug delivery system in week 24;
ix) inserting a ninth intravesical drug delivery system into the bladder of the individual in week 36, and removing the ninth intravesical drug delivery system in week 39;
x) inserting a tenth intravesical drug delivery system into the bladder of the individual in week 48, and removing the tenth intravesical drug delivery system in week 51;
xi) inserting an eleventh intravesical drug delivery system into the bladder of the individual in week 60, and removing the eleventh intravesical drug delivery system in week 63;
xii) inserting a twelfth intravesical drug delivery system into the bladder of the individual in week 72, and removing the twelfth intravesical drug delivery system in week 75;
xiii) inserting a thirteenth intravesical drug delivery system into the bladder of the individual in week 84, and removing the thirteenth intravesical drug delivery system in week 87;
xiv) inserting a fourteenth intravesical drug delivery system into the bladder of the individual in week 96, and removing the fourteenth intravesical drug delivery system in week 99;
wherein each intravesical drug delivery system comprises an intravesical device comprising about 225 mg of gemcitabine.

109. The method of claim 92, comprising:
i) deploying an intravesical drug delivery system into the bladder of the individual once every 3 weeks over approximately 6 months; followed by
ii) deploying an intravesical drug delivery system into the bladder of the individual once every 12 weeks over approximately 18 months;
wherein a total of about 14 intravesical drug delivery systems are deployed over about 2 years, and
wherein each intravesical drug delivery system is removed 3 weeks after each administration.

110. The method of claim 92, wherein the intravesical drug delivery system releases gemcitabine at a rate of about 10 mg/day to about 45 mg/day for the first seven days of administration.

111. The method of claim 92, wherein the concentration of gemcitabine in the urine reaches a maximum concentration between about 2 to about 4 days following administration of the gemcitabine.

112. The method of claim 92, wherein the intravesical drug delivery system releases gemcitabine to produce:
a mean cumulative excreted gemcitabine in urine value expressed as percentage of the administered dose from 0-168 hours from between about 36% to about 54%; and/or
a mean cumulative excreted dFdU amount in urine value expressed as percentage of the administered dose from 0-168 hours from between about 11% to about 23.3%.

113. The method of claim 92, wherein:
the intravesical drug delivery system releases about 70% of the about 225 mg gemcitabine by day 7 of administration; and/or
the intravesical drug delivery system releases about 90% of the about 225 mg gemcitabine by day 21 of administration.

114. The method of claim 92, wherein the intravesical drug delivery system releases gemcitabine during a first phase of the delivery at a first release rate followed by a second phase of the delivery having a second release rate.

115. The method of claim 92, wherein such treatment results in:
a 6-month event-free survival in the patient population between about 69% to about 95%;
a 12-month event-free survival in the patient population between about 50% to about 88%;
an 18-month event-free survival in the patient population between about 50% to about 88%; and/or
a 24-month event-free survival in the patient population between about 50% to about 88%.

116. The method of claim 92, wherein:
a) less than about 91% of individuals within the patient population experience an adverse effect;
b) less than about 35% of individuals within the patient population experience a greater than grade 3 adverse effect;
c) less than about 40% of individuals within the patient population experience micturition urgency;
d) less than about 40% of individuals within the patient population experience pollakiuria;
e) less than about 30% of individuals within the patient population experience noninfective cystitis;
f) less than about 30% of individuals within the patient population experience a urinary tract infection;
g) less than about 5% of individuals within the patient population experience pruritus;
h) less than about 5% of individuals within the patient population experience diarrhea;
i) less than about 30% of individuals within the patient population experience dysuria;
j) less than about 15% of individuals within the patient population experience hematuria;
k) less than about 10% of individuals within the patient population experience urinary tract pain;
l) less than about 10% of individuals within the patient population experience urinary retention;
m) less than about 5% of individuals within the patient population experience renal impairment; and/or
n) less than about 5% of individuals within the patient population experience urosepsis.

117. The method of claim 92, wherein:
the median duration of response (DOR) in the patient population is about 26 months;
the one-year DOR rate in the patient population is between about 50% to about 89%;
the 12-month CR rate in the patient population is between about 43% to about 79%; and/or
the 15-month CR rate in the patient population is between about 43% to about 79%.

118. The method of claim 117, wherein:
the one-year DOR rate is about 56.2%;
the 12-month CR rate in the patient population is about 45.9%; and/or
the 15-month CR rate in the patient population is about 64%.

119. A kit comprising an intravesical drug delivery system comprising about 225 mg of gemcitabine and instructions for use comprising the method of claim 92, and a urinary placement catheter.


SEQUENCE LISTING (11 sequences)

SEQ ID NO: 1 - protein, 5 aa, synthetic construct
SYAIS

SEQ ID NO: 2 - protein, 17 aa, synthetic construct
GIIPIFDTANYAQKFQG

SEQ ID NO: 3 - protein, 14 aa, synthetic construct
PGLAAAYDTGSLDY

SEQ ID NO: 4 - protein, 11 aa, synthetic construct
RASQSVRSYLA

SEQ ID NO: 5 - protein, 7 aa, synthetic construct
DASNRAT

SEQ ID NO: 6 - protein, 9 aa, synthetic construct
QQRNYWPLT

SEQ ID NO: 7 - protein, 123 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSS

SEQ ID NO: 8 - protein, 107 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIK

SEQ ID NO: 9 - protein, 449 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 268 aa, synthetic construct
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL